(19) United States
(12) Patent Application Publication
(10) Pub. No.: US 2024/0352307 A1
(43) Pub. Date: Oct. 24, 2024

(54) ACCESSING AND MODULATING THE COLOR AND VIBRANCY OF ANODICALLY COLORING ELECTROCHROMIC MOLECULES

(71) Applicant: Georgia Tech Research Corporation, Atlanta, GA (US)

(72) Inventors: Justine Sina Wagner, Atlanta, GA (US); Anna M. Osterholm, Atlanta, GA (US); Dwanleen E. Shen, Atlanta, GA (US); John Roberts Reynolds, Dunwoody, GA (US)

(21) Appl. No.: 18/630,042

(22) Filed: Apr. 9, 2024

Related U.S. Application Data
(60) Provisional application No. 63/495,329, filed on Apr. 11, 2023.

(51) Int. Cl. C09K 9/02; C07D 495/04; C09B 57/00

(52) U.S. Cl. CPC C09K 9/02; C07D 495/04; C09B 57/00; C09K 2211/1007; C09K 2211/1014; C09K 2211/1018

(57) ABSTRACT

In one aspect, the disclosure relates to anodically coloring organic electrochromic materials as discrete chromophores. The chromophores are UV absorbing in the neutral state and upon oxidation form vibrantly absorbing, colored radical cation and dication species. In an aspect, the disclosed materials include UV absorbing discrete chromophores based on a 2-thiomethyl-dioxythiophene coupled to a triphenylamine or carbazole unit. In another aspect, consideration of steric effects and tuning substituent pattern can lead to new cation radicals that are highly saturated in the colored state and have increased color vibrancy. The chromophores and materials containing the same can be used in electrochromic color-to-clear switching devices including, but not limited to, windows, displays, signs, goggles, glasses, and the like.

### CROSS REFERENCE TO RELATED APPLICATIONS

This application claims the benefit of U.S. Provisional Application No. 63/495,329 filed on Apr. 11, 2023, which is incorporated herein by reference in its entirety.

### STATEMENT REGARDING FEDERALLY SPONSORED RESEARCH OR DEVELOPMENT

This invention was made with government support under grant number FA9550-21-1-0420 awarded by Air Force Office of Scientific Research. The government has certain rights in the invention.

### BACKGROUND

While efforts in cathodically coloring systems have been focused on tuning the neutral state absorbance, understanding color tuning and vibrancy by accessing and systematically shifting the LE lower energy (LE) peak and modulating the intensity is not understood or reported in anodically coloring systems.

Electrochromism is the change of a material's color upon the application of an electrochemical potential. Easily oxidized, cathodically coloring, organic polymer electrochromes have been the focus of a large thrust of research because of their benefits of solution processability, mechanical flexibility, device bistability, and access to a variety of primary and secondary colors. Fine control of the visible spectra, and ultimately color, of these materials has been accomplished using the variables of heterocycle choice, electron rich/poor character, steric strain, and copolymerization. Applications include full color passive and active displays, energy saving tinted windows, switchable mirrors, and dimmable visors, goggles and glasses for military and/or recreational use.

The history of research in fully conjugated cathodically coloring electrochromic polymers (ECPs) has yielded materials that span the entire color palette. These polymers can be spray cast to form vividly colored films that upon oxidation become highly transmissive in the visible region. Recently, approaches towards creating black-to-transmissive electrochromic polymeric materials have been investigated through the generation of broadly absorbing copolymers or via solution mixing of polymeric inks. The latter of the two approaches allows for finer control, more accurate reproducibility, and higher contrast through the mixing of cyan, magenta, and yellow materials to create a broadly absorbing blend.

Although cathodically coloring polymers provide precise control of color and electrochemical properties, they have an inherent challenge when it comes to making improvements in contrast. In the charge neutral state conjugated ECPs absorb in the visible with a single π-π* transition (exceptions include donor-acceptor systems that have dual band absorbances and some random copolymers that have a manifold of absorbances). The oxidation states for a single polymer chain in solution consists of dual transition polarons and single transition bipolarons with discrete absorbances at longer wavelengths than the neutral polymer. In the solid-state, charged states interact with one another creating a complex system where selection rules are relaxed and more transitions are possible. This leads to the characteristically broad profile for the oxidized states of fully conjugated ECPs. An asymmetric absorption throughout the visible region, absorbing more low energy red light and transmitting more of the higher energy blue light, manifests itself in such a way that highly oxidized states of conjugated ECPs exhibit a transmissive grey-blue hue even in the highest contract materials. Reaching a color neutral and fully transmissive oxidized state across the entire visible spectrum has proven difficult due to this relaxation of the selection rules allowing broad light absorption in these materials.

What is needed is an electrochromic molecule that overcomes the drawbacks of cathodically coloring ECPs, including avoiding the near infrared (NIR) tailing observed in the ECPs. An ideal solution would offer high contrast, vibrant absorption for color mixing and color tunability in charged state. Importantly, such a molecule would be highly transmissive in the neutral state and would bring a broader set of colors when compared with anodic electrochromes reported previously. These needs and other needs are satisfied by the present disclosure.

### SUMMARY

Disclosed herein are anodically coloring organic electrochromic materials as discrete chromophores. The chromophores are UV absorbing in the neutral state and upon oxidation form vibrantly absorbing, colored radical cation and dication species. In an aspect, the disclosed materials include UV absorbing discrete chromophores based on a 2-thiomethyl-dioxythiophene coupled to a triphenylamine or carbazole unit. In another aspect, consideration of steric effects and tuning substituent pattern can lead to new cation radicals that are highly saturated in the colored state and have increased color vibrancy. The chromophores and materials containing the same can be used in electrochromic color-to-clear switching devices including, but not limited to, windows, displays, signs, goggles, glasses, and the like.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1D show ten chromophores including a 2-MeS-ethylenedioxythiophene (EDOT) coupled to either a triphenylamine or carbazole derivative with alteration in electron richness of the substituent and/or modulation of steric interactions.

FIG. 2 shows how substituent identity affects cyclic voltammetry results for selected chromophores.

FIGS. 3A-3D show how favorable and unfavorable steric interactions affect cyclic voltammetry results for selected chromophores.

FIG. 4 shows absorbance spectra of selected chromophores having substituents with different electron donating or withdrawing properties as Fe(OTf)₃ is titrated into the samples in order to induce radical cation formation.

FIGS. 5A-5D show absorbance spectra of selected chromophores having substituents with different steric interactions as Fe(OTf)₃ is titrated into the samples in order to induce radical cation formation.

FIGS. 6A-6F shows the color of radical cations generated for chromophores with selected substituents and steric interactions.

FIG. 7 shows spectroelectrohemistry data and color of the radical cations at 250 μM concentration in 0.5 M TBAPF₆ in dichloromethane.

FIGS. 8A-8E show cyclic voltammetry (CV) for selected chromophores. The molecules were present in a 250 μM concentration in 0.5 M TBAPF₆ in dichloromethane.

FIGS. 9A-9E show differential pulse voltammetry (DPV) for selected chromophores. The molecules were present in a 250 μM concentration in 0.5 M TBAPF₆ in dichloromethane.

FIGS. 10A-10E show color generation by forming the cation radicals at 125 μM concentration in dichloromethane using a Fe(OTf)₃ titrated dopant.

FIG. 11 demonstrates how shifts in vibrancy and color can be achieved by altering steric hindrance and substituent pattern.

FIGS. 12A-12E show cyclic stability analysis using 100, 500, and 1000 total scans. Scanning from −0.4 V to 1 V for (FIG. 12A) 2-MeS-EDOT-TPA-pOCH₃, (FIG. 12B) 2-MeS-EDOT-2-methoxy-TPA-pOCH₃, (FIG. 12C) 2-MeS-EDOT-2,5-dimethoxy-TPA-pOCH₃, (FIG. 12D) 2-MeS-EDOT-2,6-dimethoxy-TPA-pOCH₃, and (FIG. 12E) 2-MeS-EDOT-TPA-trimethoxy as 250 μM concentration in 0.5 M TBAPF₆ in DCM.

FIGS. 13A-13E show experimental UV-vis neutral absorbance spectrum of all chromophores: (FIG. 13A) 2-MeS-EDOT-TPA-pOCH₃, (FIG. 13B) 2-MeS-EDOT-2-methoxy-TPA-pOCH₃, (FIG. 13C) 2-MeS-EDOT-2,5-dimethoxy-TPA-pOCH₃, (FIG. 13D) 2-MeS-EDOT-2,6-dimethoxy-TPA-pOCH₃, (FIG. 13E) 2-MeS-EDOT-TPA-trimethoxy in 250 μM concentration in dichloromethane.

FIG. 14 shows chemical oxidation of 2-MeS-EDOT-TPA-trimethoxy consisting of UV-Vis spectra overlay in Fe(OTf)₃ (black line) followed by NOPF₆ oxidation (red line). Pictures demonstrate color transition from neutral to each respected charge.

FIGS. 15A-15C show UV-Vis spectra stacked of spectroelectrochemistry at long time duration (10 minutes) holding at specified potentials of (FIG. 15A) 2-MeS-EDOT-2-methoxy-TPA-pOCH₃, (FIG. 15B) 2-MeS-EDOT-2,5-TPA-pOCH₃, and (FIG. 15C) 2-MeS-EDOT-TPA-trimethoxy.

FIG. 16A shows redox transformations undertaken by a generic ACE construct where upon two successive one-electron oxidations to form a cation radical and dication charged species occurs. FIG. 16B shows a cyclic voltammogram depicting a current problem associated with the generation of separate charge states when difference in redox potentials is small and overlap. FIG. 16C is a depiction of an ideal cyclic voltammogram where two reversible redox active species have a large separation in charge state formation and form at differing oxidation potentials.

FIG. 17 shows a schematic diagram illustrating the spectra of three states in a generic ACE molecule where the neutral species (dotted line) will have a strong absorption in the UV. Upon oxidation, two major absorbance peaks are generated (solid line) associated with the cation radical having two primary energy transitions in the visible and near infrared-red (NIR) region. Upon further oxidation, a single absorbance peak attributed to the dication species evolves primarily in the visible region.

FIG. 18 shows chemical structures of five asymmetrical substituted bi-aryl (2-MeS-EDOT-TPA) chromophores with varying degrees of electron density, steric, and electrostatic interactions.

FIGS. 19A-19C show X-ray crystal structures of (FIG. 19A) 2-MeS-EDOT-TPA-pOCH₃ and (FIG. 19B) 2-MeS-EDOT-2,5-dimethoxy-TPA-pOCH₃ (variant A & B depicted), and (FIG. 19C) 2-MeS-EDOT-2,6-dimethoxy-TPA-pOCH₃ determined at 110.00(10) K. Selected atoms, the O▪▪▪H, C▪▪▪H, and O▪▪▪S bond distances (yellow line with black numerical value), and the torsion angles (green atoms highlighted with blue value) are shown. Disorder and lattice solvent molecule (for C only) were removed for clarity.

FIG. 20A shows a cyclic voltammogram of EDOT-TPA-pOCH₃ with extracted half-wave potentials (E_{1/2}) of the cation radical and dication. Inset is the abstracted differential pulse voltammogram. FIG. 20B shows DPV solution-phase oxidation potential (Eₒₓ) compared to DFT simulated adiabatic ionization potential (A_{IP}) for the first oxidation state representing the cation radical. See also Table 3 for a summary of the electrochemical properties of all five chromophores with ΔE representing potential difference between first and second oxidation peaks.

FIG. 21A shows overlaid simulated UV-Vis spectra of 2-MeS-EDOT-TPA-pOCH₃ (TPA 1), and FIG. 21B shows 2-MeS-EDOT-2,6-dimethoxy-TPA-pOCH₃ (TPA 4) for the neutral (blue line), radical cation (red line), and dication (black line). Excited analysis results for all three charges of all systems under examination. In each case, the excited state wavelength and corresponding (oscillator strength) is provided in Table 4.

FIGS. 22A-22D show (FIG. 22A) 2-MeS-EDOT-TPA-pOCH₃ (TPA 1), (FIG. 22B) 2-MeS-EDOT-2-methoxy-TPA-pOCH₃ (TPA 2), (FIG. 22C) 2-MeS-EDOT-2,5-dimethoxy-TPA-pOCH₃ (TPA 3), and (FIG. 22D) 2-MeS-EDOT-2,6-dimethoxy-TPA-pOCH₃ (TPA 4) overlay UV-Vis spectra of Fe(OTf)₃ (black line) followed by NOPF₆ oxidation (red line). Pictures demonstrate color transition from neutral to each respected charged state.

FIGS. 23A-23D show spectroelectrochemistry of (FIG. 23A) 2-MeS-EDOT-TPA-pOCH₃ (TPA 1), (FIG. 23B) 2-MeS-EDOT-2-methoxy-TPA-pOCH₃ (TPA 2), (FIG. 23C) 2-MeS-EDOT-2,5-dimethoxy-TPA-pOCH₃ (TPA 3), and FIG. 23D) 2-MeS-EDOT-2,6-dimethoxy-TPA-pOCH₃ (TPA 4) in 250 μM concentration with applied potentials from 0-1 V with 50 mV increments held for 3 minutes each. Photo demonstrates colored charge species forming on the platinum net at specified potentials.

FIGS. 24A-24B show spectroelectrochemistry of (FIG. 24A) 2-MeS-EDOT-TPA-pOCH₃ (TPA 1) and (FIG. 24B) 2-MeS-EDOT-2,6-dimethoxy-TPA-pOCH₃ (TAP 4) in 375 μM concentration specific applied potentials (with low, the two E_{1/2}, and a high potential increments held for 10 minutes each. Left gray region represents neutral, central band represents cation radical, and light right band represents dications species formation.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are anodically-coloring electrochromic molecules having chromophores that can be varied systematically to produce changes in value and hue of the colors produced when the chromophores are in the dication state. In an aspect, a first method of producing a color change is to modulate electron density such as by substituting electron donating or electron withdrawing moieties in the para position of the carbazole or triphenylamine groups in the chromophores. In another aspect, a second method of producing a color change is accomplished by substituting electron donating or electron withdrawing moieties in the para position or meta position of the triphenylamine groups in the chromophores. In another aspect, substitution of pi donors or withdrawers can be employed to produce extended pi configurations of the chromophores and to produce a rigid planar or conjugated structure or a more flexible structure enabling rotation. In still another aspect, a final method of producing a color change is by modifying steric interactions, such as by introducing favorable S—O conformational locking or by introducing unfavorable steric strain.

Controlled manipulation and separation of charges in molecular systems are at the forefront of many emerging technologies such as electrochromic displays, artificial photosynthesis, solar energy conversion, and molecular switches. The charge state of a molecule governs its physicochemical properties, such as conformation and reactivity. Specific interest has been focused on electro-active compounds due to the ability to undergo multiple and consecutive redox processes, with each redox state being distinguished by a specific property. In the field of electrochromism (EC), by controlling the redox states, structuring their internal electronic interactions, and switching charge states, a range of optical (color) states can be attained and implemented into device applications.

Among the most representative examples of organic EC systems are viologen compounds and their analogues due to their advantageous dicationic oxidation state, excellent electron affinity, stability, and electrochemically reversible redox activity. Viologens (V²⁺) are planar, colorless, and transmissive dicationic ϕ-conjugated acceptors that undergo two successive one-electron reductions yielding cation radicals (V·⁻) and a neutral quinoid form (V⁰), both of which are colored species. Many synthetic modifications have been applied to increase redox stability such as bridging groups in the bipyridine bay and incorporating substituents.

An alternative and promising system consists of anodically coloring electrochromics (ACEs) where there has been significant progress in understanding how absorption in the charged state is controlled. In 2019, it was demonstrated that cross-conjugation can controllably tune the electronic energy levels of the cation radical independently of the neutral state by adjusting the electron rich character of substituents at the meta position of a bi-aryl 2-thiomethyl-3,4-ethylenedioxythiophene coupled to a 4-methoxybenzene derivative (EDOT-Ph). In 2022, an alternative structural motif consisting of a thioalkyl-substituted bis(3,4-ethylenedioxythiophene)-1,4-phenylene (BEDOT-Ph) with varying alkoxy group placements was reported. This deepened the understanding of how to synthetically control the optoelectronic properties as a function of charged state to fine-tune hue and saturation for the color blue. Furthermore, this approach was recently expanded to vary the electronic and structural effects of BEDOT-Ph derivatives through substituent alterations, allowing accession of colors not previously attainable in ACE molecules.

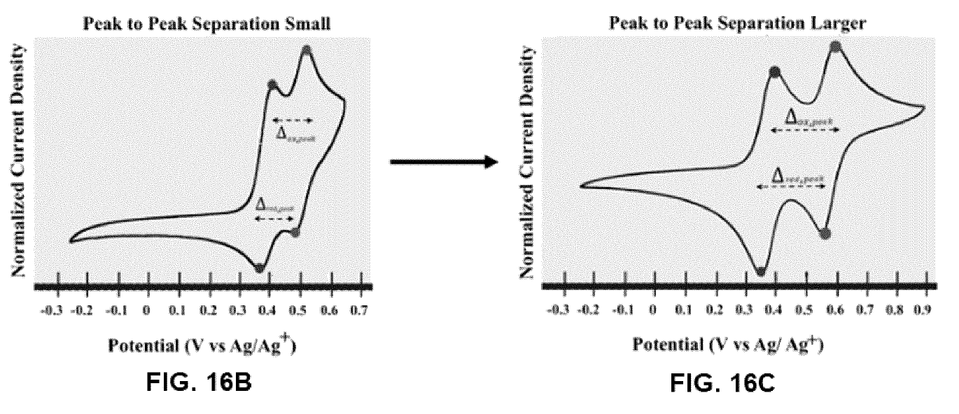

ACE molecules differ from viologens in charged state formation, as illustrated in FIG. 16A; where viologens are cathodic in nature, ACE molecules are anodic. An ACE molecule starts as colorless and transmissive in the neutral state. Upon oxidation, a colored species is generated when forming a cation radical. Further oxidation results in the formation of a dicationic charged species, leading to a new sequential color. In previous investigations, it was uncovered through the study of the redox properties of ACE chromophores using cyclic voltammetry, that sequential oxidation processes representing cation radical and dication formation are close in potential or lack a wide charge state separation, as depicted in FIG. 16B. Disclosed herein is a method to increase the distance between these redox processes, as depicted in FIG. 16C, where two reversible active species are generated at well-separated oxidation potentials. Accessing this will allow for direct control of the sequentially charged species formation and, thus, color control.

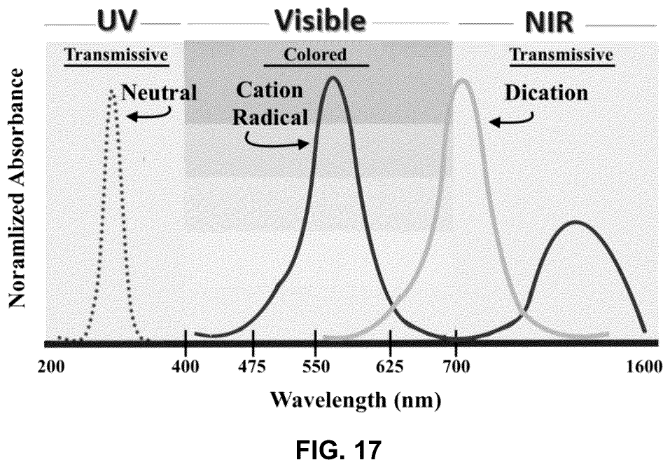

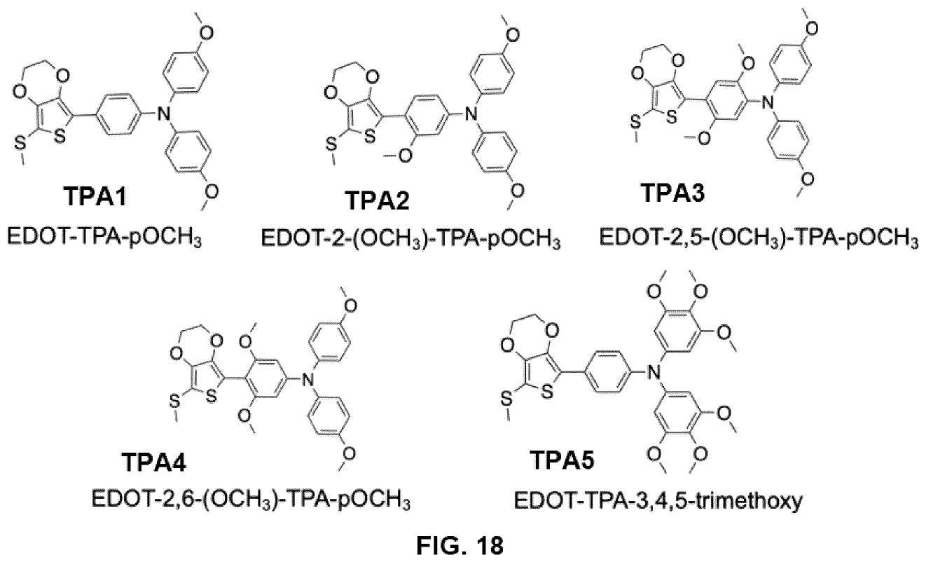

As mentioned, the ability to tailor and control colors, switching from colorless and transmissive to a high-contrast color, is a desirable feature for many applications. The coloration mechanism in electrochromic systems can be probed by comparing the evolution of the spectral response. As shown schematically in FIG. 17, as the applied potential increases, the neutral band (also known as π-π*) represented by the dotted line fully in the ultraviolet, will gradually decrease in intensity and new absorption peaks ascribed to the cation radical (indicated by the connected line) evolve with a high energy (HE) transition emerging in the visible region and a lower energy (LE) transition in the NIR. Further incremental increase in potential will generate the dication. In previous investigations, having sequential redox features that are not well-separated resulted in the simultaneous formation of the cation radical and dication species at specific high oxidation potentials, resulting in both charged states forming. This, in turn, lead to the inability to control the color attained, causing a mixture or blend of two charged species which creates a new color.

Herein are introduced new ACE designs comprised of a triphenylamine donor coupled to an EDOT. The goals are to maintain a highly transmissive and colorless neutral state while gaining a systematic understanding of how to control and increase the potential separation (ΔE) of each successive charged state. Fundamentally, it is essential to comprehend the dynamics of multiple redox reactions and the associated structural evolutions during the redox reactions. In addition, by controlling the dynamic formation of charged species, the generation of vibrantly colored species can be achieved and expand the ACE color palette. A new class of ACE electrochromes has been synthesized, systematically varying electron richness and steric interactions that is observed computationally by DFT calculations and experimentally by single crystal X-ray diffraction (hereafter, SCXRD). This approach enabled access to both a cation radical and dication state using two chemical oxidants and maintain control over redox properties, separation and control of charge species, along with enhancing the color contrast and saturation in the cation radical state. Computational models and calculations were generated using TD-DFT at the mPW1 PBE/cc-PVDZ level. These results are used in tandem with those produced experimentally to comprehend how modulating electron richness and steric interactions affects system color. Specifically, excited state analysis is performed to elucidate how substituent identity affects the neutral, radical cation and dication energy peaks in the visible and near infrared, and thereby the resulting color.

In one aspect, the anodically-coloring electrochromic molecules disclosed herein can have a structure according to Formula I:

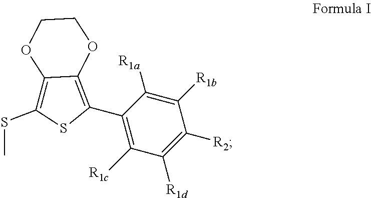

wherein R₁ₐ, R_{1b}, R_{1c}, and R_{1d} are independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino, and
wherein R₂ is selected from a substituted or unsubstituted triphenylamine (TPA) and substituted or unsubstituted carbazole (Cz).

In another aspect, the anodically-coloring electrochromic molecule has a structure according to Formula Ia (triphenylamine or TPA derivative) or Formula Ib (carbazole or Cz derivative):

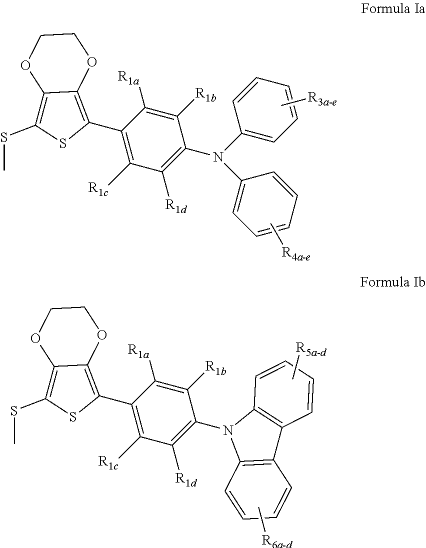

wherein, when the anodically-coloring electrochromic molecule has the structure according to Formula Ia, each of R₃ₐ₋ₑ and R₄ₐ₋ₑ is independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino; and
wherein, when the anodically-coloring electrochromic molecule has the structure according to Formula Ib, each of R_{5a-d} and R_{6a-d} is independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino.

In a further aspect, each of R₁ₐ, R_{1b}, R_{1c}, and R_{1d} can independently be selected from —CN, —NO₂, —CF₃, —F, —Cl, hydrogen, —CH₃, —OCH₃, —SCH₃, —NH₂, and —N(CH₃)₂. In another aspect, when the anodically-coloring electrochromic molecule has the structure according to Formula Ia, each of R₃ₐ₋ₑ and R₄ₐ₋ₑ is independently selected from —CN, —NO₂, —CF₃, —F, —Cl, hydrogen, —CH₃, —OCH₃, —SCH₃, —NH₂, and —N(CH₃)₂; and, when the anodically-coloring electrochromic molecule has the structure according to Formula Ib, each of R_{5a-d} and R_{6a-d} is independently selected from —CN, —NO₂, —CF₃, —F, —Cl, hydrogen, —CH₃, —OCH₃, —SCH₃, —NH₂, and —N(CH₃)₂

However, in some aspects, the para substituent may not be hydrogen. Without wishing to be bound by theory, hydrogen in the para position may cause the molecule to polymerize due to change in spin density and reactivity. In one aspect, in the anodically-coloring electrochromic molecule, each of R₁ₐ, R_{1b}, R_{1c}, and R_{1d} are hydrogen; or R₁ₐ, R_{1b}, and R_{1d} are hydrogen and wherein R_{1c} is methoxy; or R₁ₐ and R_{1d} are hydrogen and wherein R_{1b} and R_{1c} are methoxy; or R_{1b} and R_{1d} are hydrogen and wherein R₁ₐ and R_{1c} are methoxy.

In some aspects, the anodically-coloring electrochromic molecule has Formula Ia, wherein at least one of R₃ₐ—R₃ₑ is methoxy, wherein at least one of R₄ₐ—R₄ₑ is methoxy, and wherein any of R₃ₐ—R₃ₑ and any of R₄ₐ—R₄ₑ that are not methoxy are hydrogen; or wherein at least one of R₃ₐ—R₃ₑ is —SCH₃, wherein at least one of R₄ₐ—R₄ₑ is —SCH₃, and wherein any of R₃ₐ—R₃ₑ and any of R₄ₐ—R₄ₑ that are not —SCH₃ are hydrogen; or wherein at least one of R₃ₐ—R₃ₑ is —CN, wherein at least one of R₄ₐ—R₄ₑ is —CN, and wherein any of R₃ₐ—R₃ₑ and any of R₄ₐ—R₄ₑ that are not —CN are hydrogen; or wherein at least three of R₃ₐ—R₃ₑ are methoxy, wherein at least three of R₄ₐ—R₄ₑ are methoxy, and wherein any of R₃ₐ—R₃ₑ and any of R₄ₐ—R₄ₑ that are not methoxy are hydrogen.

In an alternative aspect, the anodically-coloring electrochromic molecule has Formula Ib, wherein at least one of R₅ₐ—R_{5d} is methoxy, wherein at least one of R₆ₐ—R_{6d} is methoxy, and wherein any of R₅ₐ—R_{5d} and any of R₆ₐ—R_{6d} that are not methoxy are hydrogen; or wherein at least one of R₅ₐ—R_{5d} is —SCH₃, wherein at least one of R₆ₐ—R_{6d} is —SCH₃, and wherein any of R₅ₐ—R_{5d} and any of R₆ₐ—R_{6d} that are not —SCH₃ are hydrogen; or wherein at least one of R₅ₐ—R_{5d} is —CN, wherein at least one of R₆ₐ—R_{6d} is —CN, and wherein any of R₅ₐ—R_{5d} and any of R₆ₐ—R_{6d} that are not —CN are hydrogen.

In any of these aspects, the anodically-coloring electrochromic molecule can have one of the following structures:

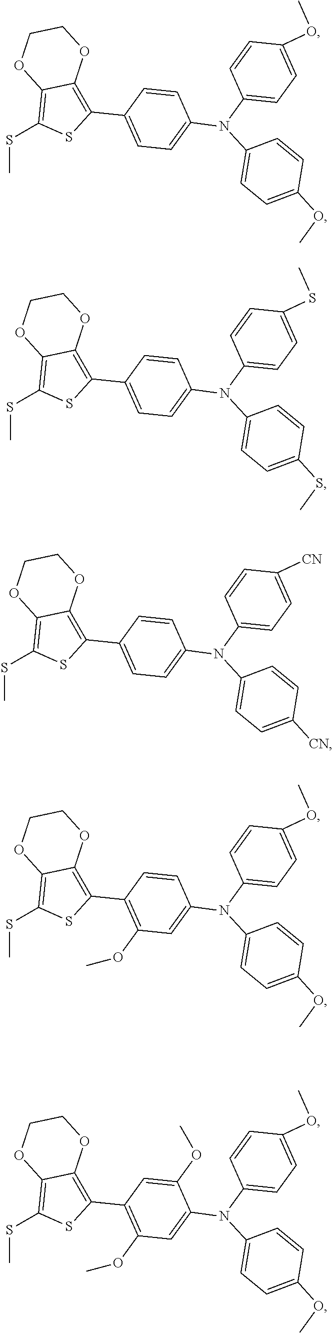

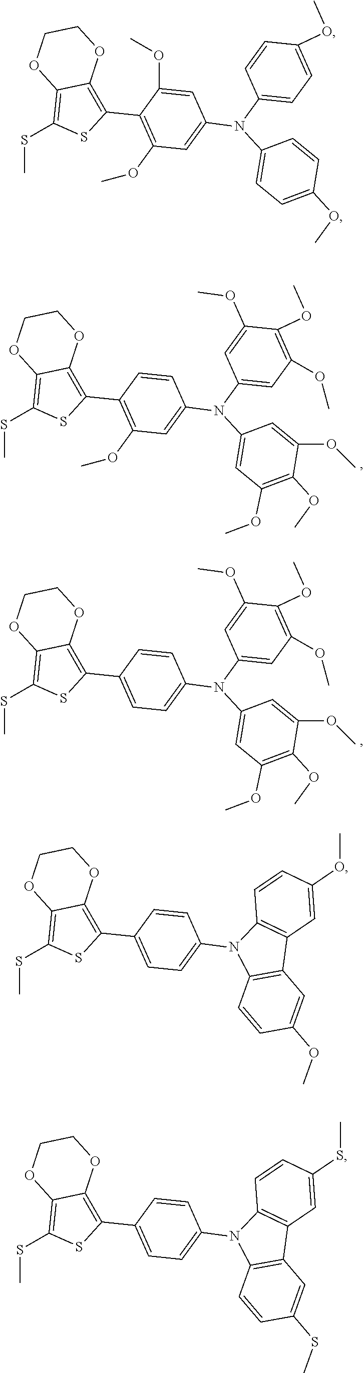

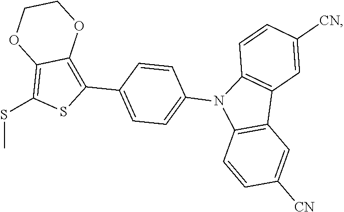 [SMILES: CSc1sc(-c2ccc(-n3c4ccc(C#N)cc4c4cc(C#N)ccc43)cc2)c2c1OCCO2]

or any combination thereof.

In an aspect, the anodically-coloring electrochromic molecule can be TPA 1, TPA 2, TPA 3, TPA 4, or TPA 5.

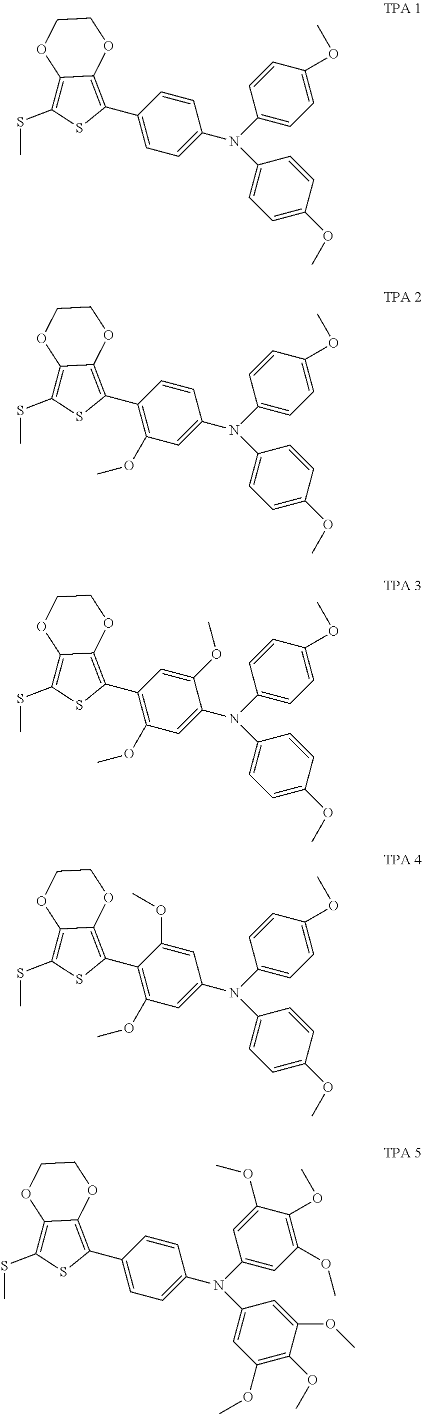

In a further aspect, the anodically-coloring electrochromic molecule can have a first color in its neutral state and a second color after being oxidized. In an aspect, for the purposes of this disclosure, a first “color” should be interpreted to include a visible color, or, in the alternative, a transparent or colorless state. As used herein, “transparent” can be defined as completely colorless or as having a slight yellow color wherein at least 95% of white light having a wavelength of from about 380 nm to about 780 nm passes through.

In one aspect, the anodically-coloring electrochromic molecule can be oxidized by contact with an oxidant, by application of an oxidation potential, or any combination thereof.

In some aspects, the oxidant can be Fe(OTf)₃, iron (III) perchlorate, iron (III) chloride, iron (III) tosylate, nitrosonium hexafluorophosphate, 2,3,5,6-tetrafluoro-7,7,8,8-tetracyano-quinodimethane (F4TCNQ), tetracyanoquinodimethane (TCNQ), tetracyanoethylene (TCNE), [3]-radialene, tris(4-bromophenyl)ammoniumyl hexachloroantimonate (magic blue), HCl, H₂SO₄, zinc, borane, a carbonium compound, an oxonium compound, iodine, arsenic pentafluoride, or any combination thereof.

Also disclosed herein are electrochromic devices including at least:
(a) a first electrode;
(b) a second electrode;
(c) an electrolyte in contact with the first electrode and the second electrode; and
(d) an anodically-coloring electrochromic molecule as disclosed herein.

In another aspect, in the disclosed electrochromic devices, the anodically-coloring electrochromic molecule can have a first color in its neutral state and a second color after being oxidized, where a first “color” is defined as disclosed above (i.e., to include a transparent or colorless state). In some aspects, the anodically-coloring electrochromic molecule can be dispersed within the electrolyte, or can be covalently attached to a surface of the first electrode that is in contact with the electrolyte.

In some aspects, the electrolyte can be is selected from the group consisting of an organic electrolyte, an aqueous electrolyte, a biological electrolyte, a solid state electrolyte, and any combination thereof. In another aspect, the electrolyte includes an anion selected from: F⁻, Cl⁻, Br⁻, ClO₄⁻, PF₆⁻, TFSI⁻, and CFSO₃⁻, and a cation selected from Na⁺, K⁺, Li⁺, tetrabutylammonium (TBA⁺), or any combination thereof, or the electrolyte can be selected from the group consisting of sodium chloride, potassium chloride, tetrabutylammonium hexafluorophosphate (TBAPF₆), and lithium bis(trifluoromethylsulfonyl)imide (LiBTI), and any combination thereof. In a further aspect, when the electrolyte is or includes an ionic liquid, the ionic liquid can be selected from EMIM BF₄, EMIM FSI, EMIM TFSI, BMIM BF₄, FMIM TFSI, BMPyrr TFSI, BMPyrr FSI or any combination thereof.

In one aspect, one or both of the first electrode and the second electrode are selected from the group consisting of transparent conducting oxide coated electrode, a conductive polymer coated electrode, a metal grid electrode, a carbon nanotube electrode, a metal film electrode, and any combination thereof; or are selected from the group consisting of a graphene electrode, an indium-tin-oxide electrode, a PEDOT:PSS electrode, a high surface area mesoporous metal oxide electrode, and any combination thereof. In some aspects, the second electrode can include a cathodically coloring electrochromic molecule that is capable of being reduced when a voltage is applied, an optically inactive molecule that is capable of being reduced when a voltage is applied, or any combination thereof. In one aspect, the high surface area mesoporous metal oxide electrode is capable of storing charge capacitatively.

Also disclosed are articles including the disclosed electrochromic devices. In one aspect, the article can be a window, a display, a sign, goggles, or glasses.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a chromophore,” “a substituent,” or “a cation,” includes, but is not limited to, mixtures or combinations of two or more such chromophores, substituents, or cations, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, ‘less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, ‘greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a catalyst refers to an amount that is sufficient to achieve the desired lowering of the activation energy barrier for a reaction to proceed and/or allows a reaction to proceed more rapidly. The specific level in terms of wt % in a composition required as an effective amount will depend upon a variety of factors including the amount and type of solvent, reaction temperature, steric hindrance of reactants and/or transition states, and the like.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR⁰²; —C(S)NR^{∘}₂; —C(S)SR^{∘}; —(CH₂)₀₋₄OC(O)NR⁰²; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid. In some aspects, in the disclosed compounds, intermolecular non-covalent interactions can occur. Further in these aspects, these intermolecular non-covalent interactions can lock the anodically coloring electrochromic chromophores into a planar configuration. In one aspect, these non-covalent attractive interactions can occur over shorter distances than the van der Waals radii of the substituents. In a further aspect, this effect can be especially prominent between S—O and S—F substituents.

It is also appreciated that certain compounds described herein can be present as an equilibrium of tautomers. For example, ketones with an α-hydrogen can exist in an equilibrium of the keto form and the enol form.

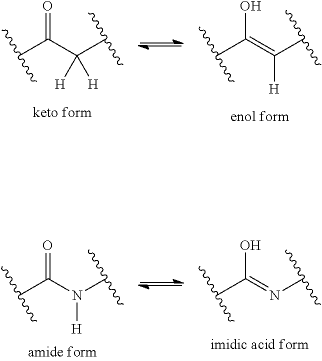 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

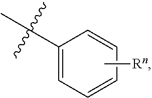

which is understood to be equivalent to a formula:

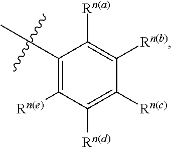

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. An anodically-coloring electrochromic molecule having a structure according to Formula I:

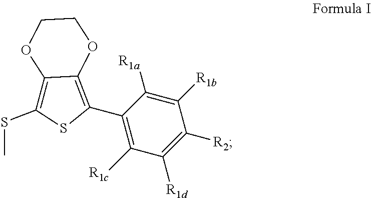

wherein R₁ₐ, R_{1b}, R_{1c}, and R_{1d} are independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino, and
wherein R₂ is selected from a substituted or unsubstituted triphenylamine (TPA) and substituted or unsubstituted carbazole (Cz)

Aspect 2. The anodically-coloring electrochromic molecule of aspect 1, wherein the anodically-coloring electrochromic molecule has a structure according to Formula Ia or Formula Ib:

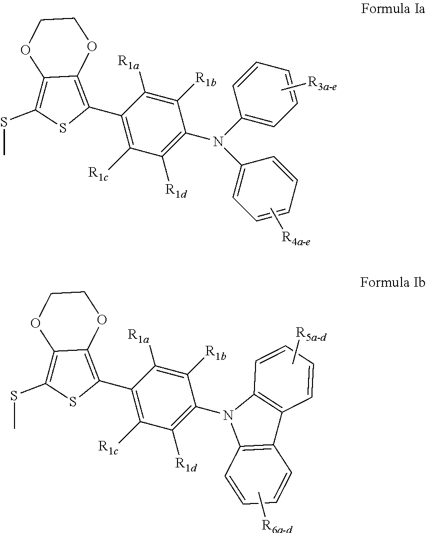

wherein, when the anodically-coloring electrochromic molecule has the structure according to Formula Ia, each of R₃ₐ₋ₑ and R₄ₐ₋ₑ is independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino; and
wherein, when the anodically-coloring electrochromic molecule has the structure according to Formula Ib, each of R_{5a-d} and R_{6a-d} is independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino.

Aspect 3. The anodically-coloring electrochromic molecule of aspect 1 or 2, wherein each of R₁ₐ, R_{1b}, R_{1c}, and R_{1d} are independently selected from —CN, —NO₂, —CF₃, —F, —Cl, hydrogen, —CH₃, —OCH₃, —SCH₃, —NH₂, and —N(CH₃)₂.

Aspect 4. The anodically-coloring electrochromic molecule of any one of aspects 1-3, wherein, when the anodically-coloring electrochromic molecule has the structure according to Formula Ia, each of R₃ₐ₋ₑ and R₄ₐ₋ₑ is independently selected from —CN, —NO₂, —CF₃, —F, —Cl, hydrogen, —CH₃, —OCH₃, —SCH₃, —NH₂, and —N(CH₃)₂; and wherein, when the anodically-coloring electrochromic molecule has the structure according to Formula Ib, each of R_{5a-d} and R_{6a-d} is independently selected from —CN, —NO₂, —CF₃, —F, —C, hydrogen, —CH₃, —OCH₃, —SCH₃, —NH₂, and —N(CH₃)₂

Aspect 5. The anodically-coloring electrochromic molecule of any one of aspects 1-4, wherein each of R₁ₐ, R_{1b}, R_{1c}, and R_{1d} are hydrogen.

Aspect 6. The anodically-coloring electrochromic molecule of any one of aspects 1-4, wherein R₁ₐ, R_{1b}, and R_{1d} are hydrogen and wherein R₁, is methoxy.

Aspect 7. The anodically-coloring electrochromic molecule of any one of aspects 1-4, wherein R₁ₐ and R_{1d} are hydrogen and wherein R_{1b} and R_{1c} are methoxy.

Aspect 8. The anodically-coloring electrochromic molecule of any one of aspects 1-4, wherein R_{1b} and R_{1d} are hydrogen and wherein R₁ₐ and R_{1c} are methoxy.

Aspect 9. The anodically-coloring electrochromic molecule of any one of aspects 2-8, wherein the anodically-coloring electrochromic molecule is Formula Ia;
wherein at least one of R₃ₐ—R₃ₑ is methoxy;
wherein at least one of R₄ₐ—R₄ₑ is methoxy; and
wherein any of R₃ₐ—R₃ₑ and any of R₄ₐ—R₄ₑ that are not methoxy are hydrogen.

Aspect 10. The anodically-coloring electrochromic molecule of any one of aspects 2-8, wherein the anodically-coloring electrochromic molecule is Formula Ia;
wherein at least one of R₃ₐ—R₃ₑ is —SCH₃;
wherein at least one of R₄ₐ—R₄ₑ is —SCH₃; and
wherein any of R₃ₐ—R₃ₑ and any of R₄ₐ—R₄ₑ that are not —SCH₃ are hydrogen.

Aspect 11. The anodically-coloring electrochromic molecule of any one of aspects 2-8, wherein the anodically-coloring electrochromic molecule is Formula Ia;
wherein at least one of R₃ₐ—R₃ₑ is —CN;
wherein at least one of R₄ₐ-R₄ₑ is —CN; and
wherein any of R₃ₐ—R₃ₑ and any of R₄ₐ—R₄ₑ that are not —CN are hydrogen.

Aspect 12. The anodically-coloring electrochromic molecule of any one of aspects 2-8, wherein the anodically-coloring electrochromic molecule is Formula Ia;
wherein at least three of R₃ₐ—R₃ₑ are methoxy;
wherein at least three of R₄ₐ—R₄ₑ are methoxy; and
wherein any of R₃ₐ—R₃ₑ and any of R₄ₐ—R₄ₑ that are not methoxy are hydrogen.

Aspect 13. The anodically-coloring electrochromic molecule of any one of aspects 2-8, wherein the anodically-coloring electrochromic molecule is Formula Ib;
wherein at least one of R₅ₐ—R_{5d} is methoxy;
wherein at least one of R₆ₐ—R_{6d} is methoxy; and
wherein any of R₅ₐ—R_{5d} and any of R₆ₐ—R_{6d} that are not methoxy are hydrogen.

Aspect 14. The anodically-coloring electrochromic molecule of any one of aspects 2-8, wherein the anodically-coloring electrochromic molecule is Formula Ib;
wherein at least one of R₅ₐ—R_{5d} is —SCH₃;
wherein at least one of R₆ₐ—R_{6d} is —SCH₃; and
wherein any of R₅ₐ—R_{5d} and any of R₆ₐ—R_{6d} that are not —SCH₃ are hydrogen.

Aspect 15. The anodically-coloring electrochromic molecule of any one of aspects 2-8, wherein the anodically-coloring electrochromic molecule is Formula Ib;
wherein at least one of R₅ₐ—R_{5d} is —CN;
wherein at least one of R₆ₐ—R_{6d} is —CN; and
wherein any of R₅ₐ—R_{5d} and any of R₆ₐ—R_{6d} that are not —CN are hydrogen.

Aspect 16. The anodically-coloring electrochromic molecule of any one of aspects 1-15, wherein the anodically-coloring electrochromic molecule is selected from:

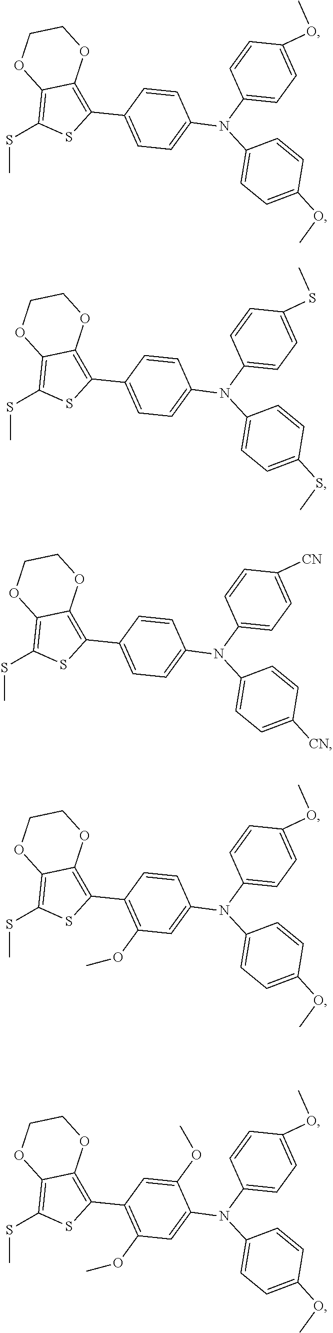

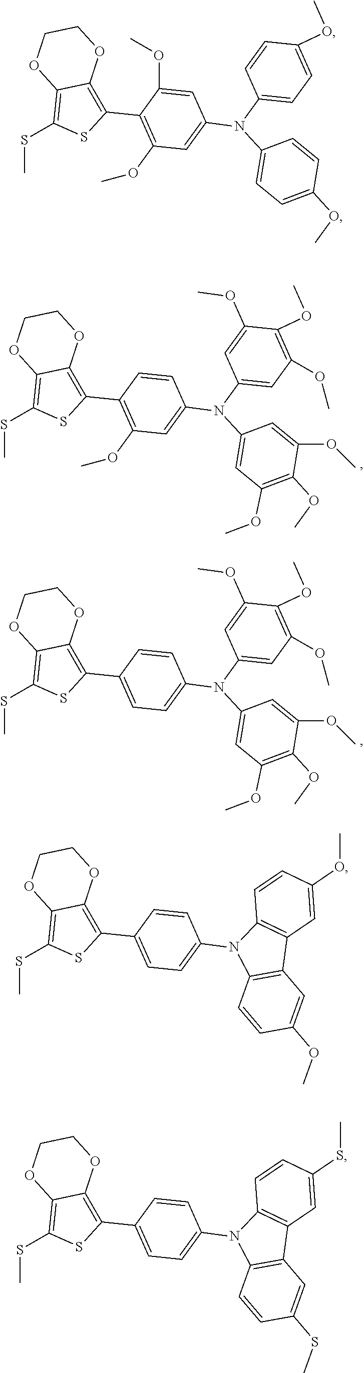

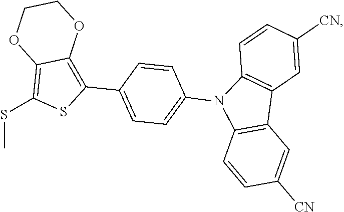 [SMILES: CSc1sc(-c2ccc(-n3c4ccc(C#N)cc4c4cc(C#N)ccc43)cc2)c2c1OCCO2]

or any combination thereof.

Aspect 17. The anodically-coloring electrochromic molecule of any one of aspects 1-16, wherein the anodically-coloring electrochromic molecule comprises a first color in its neutral state and a second color after being oxidized.

Aspect 18. The anodically-coloring electrochromic molecule of aspect 17, wherein the first color is transparent.

Aspect 19. The anodically-coloring electrochromic molecule of aspect 17 or 18, wherein the anodically-coloring electrochromic molecule is oxidized by contact with an oxidant, by application of an oxidation potential, or any combination thereof.

Aspect 20. The anodically-coloring electrochromic molecule of aspect 19, wherein the oxidant comprises Fe(OTf)₃, iron (III) perchlorate, iron (III) chloride, iron (III) tosylate, nitrosonium hexafluorophosphate, 2,3,5,6-tetrafluoro-7,7,8,8-tetracyano-quinodimethane (F4TCNQ), tetracyanoquinodimethane (TCNQ), tetracyanoethylene (TCNE), [3]-radialene, tris(4-bromophenyl)ammoniumyl hexachloroantimonate (magic blue), HCl, H₂SO₄, zinc, borane, a carbonium compound, an oxonium compound, iodine, arsenic pentafluoride, or any combination thereof.

Aspect 21. An electrochromic device comprising:
(a) a first electrode;
(b) a second electrode;
(c) an electrolyte in contact with the first electrode and the second electrode; and
(d) an anodically-coloring electrochromic molecule having a structure according to Formula I:

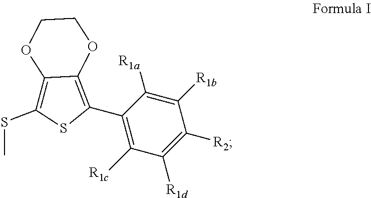

wherein R₁ₐ, R_{1b}, R_{1c}, and R_{1d} are independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino, and
wherein R₂ is selected from a substituted or unsubstituted triphenylamine (TPA) and substituted or unsubstituted carbazole (Cz).

Aspect 22. The electrochromic device of aspect 21, wherein the anodically-coloring electrochromic molecule has a structure according to Formula Ia or Formula Ib:

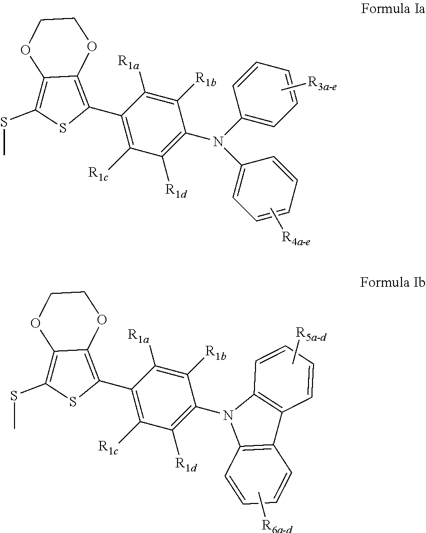

wherein, when the anodically-coloring electrochromic molecule has the structure according to Formula Ia, each of R₃ₐ₋ₑ and R₄ₐ₋ₑ is independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino; and
wherein, when the anodically-coloring electrochromic molecule has the structure according to Formula Ib, each of R_{5a-d} and R_{6a-d} is independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino.

Aspect 23. The electrochromic device of aspect 21 or 22, wherein each of R₁ₐ, R_{1b}, R_{1c}, and R_{1d} are independently selected from —CN, —NO₂, —CF₃, —F, —Cl, hydrogen, —CH₃, —OCH₃, —SCH₃, —NH₂, and —N(CH₃)₂.

Aspect 24. The electrochromic device of aspect 22 or 23, wherein, when the anodically-coloring electrochromic molecule has the structure according to Formula Ia, each of R₃ₐ₋ₑ and R₄ₐ₋ₑ is independently selected from —CN, —NO₂, —CF₃, —F, —Cl, hydrogen, —CH₃, —OCH₃, —SCH₃, —NH₂, and —N(CH₃)₂; and

wherein, when the anodically-coloring electrochromic molecule has the structure according to Formula Ib, each of R_{5a-d} and R_{6a-d} is independently selected from —CN, —NO₂, —CF₃, —F, —C, hydrogen, —CH₃, —OCH₃, —SCH₃, —NH₂, and —N(CH₃)₂.

Aspect 25. The electrochromic device of any one of aspects 21-24, wherein each of R₁ₐ, R_{1b}, R_{1c}, and R_{1d} are hydrogen.

Aspect 26. The electrochromic device of any one of aspects 21-24, wherein R₁ₐ, R_{1b}, and R_{1d} are hydrogen and wherein R₁, is methoxy.

Aspect 27. The electrochromic device of any one of aspects 21-24, wherein R₁ₐ and R_{1d} are hydrogen and wherein R_{1b} and R_{1c} are methoxy.

Aspect 28. The electrochromic device of any one of aspects 21-24, wherein R_{1b} and R_{1d} are hydrogen and wherein R₁ₐ and R_{1c} are methoxy.

Aspect 29. The electrochromic device of any one of aspects 22-28, wherein the anodically-coloring electrochromic molecule is Formula 1a;
wherein at least one of R₃ₐ—R₃ₑ is methoxy;
wherein at least one of R₄ₐ—R₄ₑ is methoxy; and
wherein any of R₃ₐ—R₃ₑ and any of R₄ₐ—R₄ₑ that are not methoxy are hydrogen.

Aspect 30. The electrochromic device of any one of aspects 22-28, wherein the anodically-coloring electrochromic molecule is Formula 1a;
wherein at least one of R₃ₐ—R₃ₑ is —SCH₃;
wherein at least one of R₄ₐ-R₄ₑ is —SCH₃; and
wherein any of R₃ₐ—R₃ₑ and any of R₄ₐ—R₄ₑ that are not —SCH₃ are hydrogen.

Aspect 31. The electrochromic device of any one of aspects 22-28, wherein the anodically-coloring electrochromic molecule is Formula 1a;
wherein at least one of R₃ₐ—R₃ₑ is —CN;
wherein at least one of R₄ₐ-R₄ₑ is —CN; and
wherein any of R₃ₐ—R₃ₑ and any of R₄ₐ—R₄ₑ that are not —CN are hydrogen.

Aspect 32. The electrochromic device of any one of aspects 22-28, wherein the anodically-coloring electrochromic molecule is Formula 1a;
wherein at least three of R₃ₐ—R₃ₑ are methoxy;
wherein at least three of R₄ₐ—R₄ₑ are methoxy; and
wherein any of R₃ₐ—R₃ₑ and any of R₄ₐ—R₄ₑ that are not methoxy are hydrogen.

Aspect 33. The electrochromic device of any one of aspects 22-28, wherein the anodically-coloring electrochromic molecule is Formula Ib;
wherein at least one of R₅ₐ—R_{5d} is methoxy;
wherein at least one of R₆ₐ—R_{6d} is methoxy; and
wherein any of R₅ₐ—R_{5d} and any of R₆ₐ—R_{6d} that are not methoxy are hydrogen.

Aspect 34. The electrochromic device of any one of aspects 22-28, wherein the anodically-coloring electrochromic molecule is Formula Ib;
wherein at least one of R₅ₐ—R_{5d} is —SCH₃;
wherein at least one of R₆ₐ—R_{6d} is —SCH₃; and
wherein any of R₅ₐ—R_{5d} and any of R₆ₐ—R_{6d} that are not —SCH₃ are hydrogen.

Aspect 35. The electrochromic device of any one of aspects 22-28, wherein the anodically-coloring electrochromic molecule is Formula Ib;
wherein at least one of R₅ₐ—R_{5d} is —CN;
wherein at least one of R₆ₐ—R_{6d} is —CN; and
wherein any of R₅ₐ—R_{5d} and any of R₆ₐ—R_{6d} that are not —CN are hydrogen.

Aspect 36. The electrochromic device of any one of aspects 21-35, wherein the anodically-coloring electrochromic molecule is selected from:

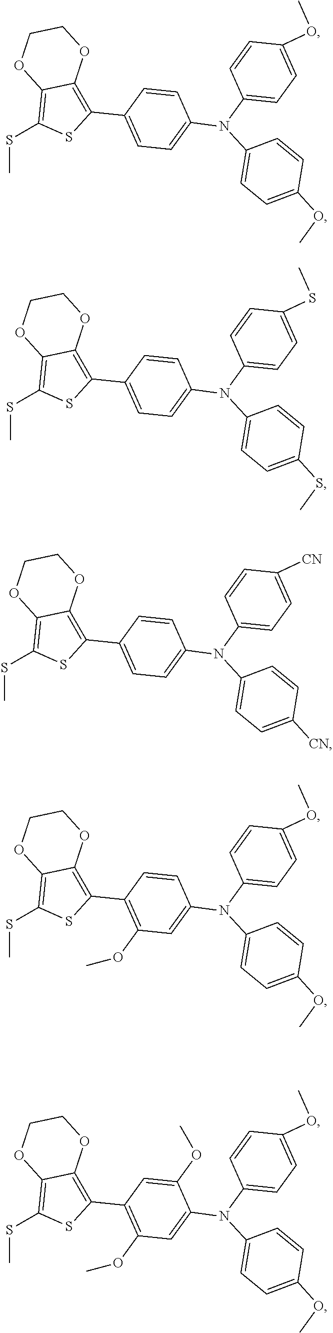

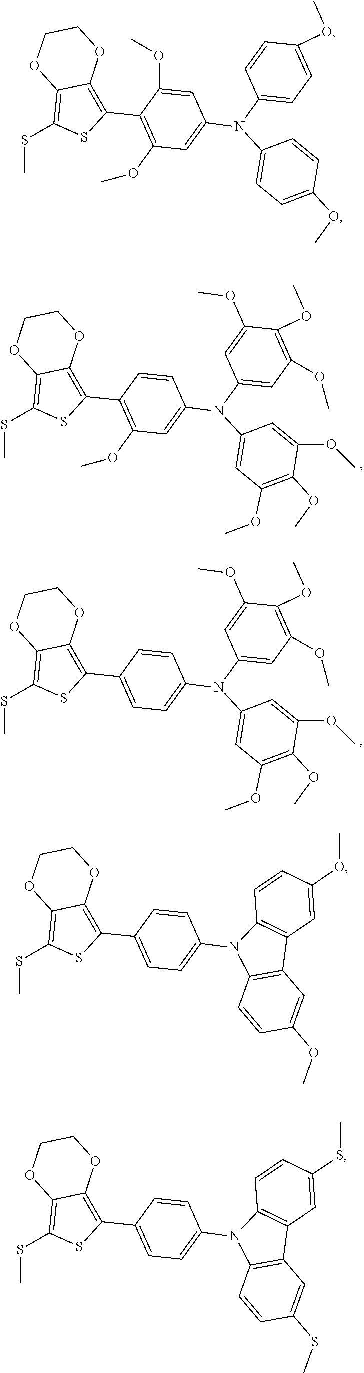

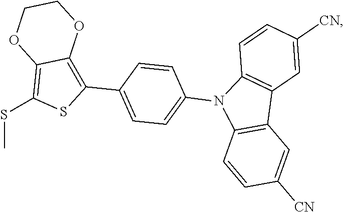 [SMILES: CSc1sc(-c2ccc(-n3c4ccc(C#N)cc4c4cc(C#N)ccc43)cc2)c2c1OCCO2]

or any combination thereof.

Aspect 37. The electrochromic device of any one of aspects 21-36, wherein the anodically-coloring electrochromic molecule comprises a first color in its neutral state and a second color after being oxidized.

Aspect 38. The electrochromic device of aspect 37, wherein the first color is transparent.

Aspect 39. The electrochromic device according to any one of aspects 21-38, wherein the anodically-coloring electrochromic molecule is dispersed within the electrolyte.

Aspect 40. The electrochromic device according to any one of aspects 21-38, wherein the anodically-coloring electrochromic molecule is covalently attached to a surface of the first electrode that is in contact with the electrolyte.

Aspect 41. The electrochromic device according to any one of aspects 21-40, wherein the electrolyte is selected from the group consisting of an organic electrolyte, an aqueous electrolyte, a biological electrolyte, an ionic liquid electrolyte, a solid state electrolyte, and any combination thereof.

Aspect 42. The electrochromic device of aspect 41, wherein the electrolyte comprises an anion selected from: F⁻, Cl⁻, Br⁻, ClO₄⁻, PF₆⁻, TFSI⁻, and CFSO₃⁻, and a cation selected from Na⁺, K⁺, Li⁺, tetrabutylammonium (TBA⁺), or any combination thereof.

Aspect 43. The electrochromic device according to any one of aspects 21-42, wherein the electrolyte comprises sodium chloride, potassium chloride, triethylamine, tetrabutylammonium hexafluorophosphate (TBAPF₆), tetrabutylammonium tetrafluoroborate (TBABF₄), lithium bis(trifluoromethylsulfonyl)imide (LiBTI), another tetraalkylammonium electrolyte, or any combination thereof.

Aspect 44. The electrochromic device of aspect 41, wherein the ionic liquid electrolyte comprises EMIM BF₄, EMIM FSI, EMIM TFSI, BMIM BF₄, FMIM TFSI, BMPyrr TFSI, BMPyrr FSI or any combination thereof.

Aspect 45. The electrochromic device according to any one of aspects 21-44, wherein one or both of the first electrode and the second electrode are selected from the group consisting of transparent conducting oxide coated electrode, a conductive polymer coated electrode, a metal grid on electrode, a carbon nanotube on electrode, a metal film on electrode, or any combination thereof.

Aspect 46. The electrochromic device according to any one of aspects 21-44, wherein one or both of the first electrode and the second electrode are selected from the group consisting of a graphene electrode, an indium-tin-oxide electrode, a PEDOT:PSS electrode, a high-surface area mesoporous metal oxide electrode, or any combination thereof.

Aspect 47. The electrochromic device according to any one of aspects 21-46, wherein the second electrode comprises a cathodically coloring electrochromic molecule that is capable of being reduced when a voltage is applied, an optically inactive molecule that is capable of being reduced when a voltage is applied, or any combination thereof.

Aspect 48. The electrochromic device according to aspect 46 or 47, wherein the high-surface area mesoporous metal oxide electrode is capable of storing charge capacitatively.

Aspect 49. An article comprising the electrochromic device of any one of aspects 21-48.

Aspect 50. The article of aspect 49, wherein the article comprises a window, a display, a sign, goggles, or glasses.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Strategy for Substituent Selection and Synthetic Methods

An ethylenedioxythiophene (EDOT) is coupled to a second electron rich moiety consisting of either a triphenylamine (TPA) or a carbazole (Cz) derivative. Electron donating and withdrawing substituents, labeled as R₁, R₂, or R₃, were installed on either the phenylene ring adjacent to the EDOT, or on the phenyl pendant rings farthest away from the EDOT. The location of the substituents directly affects both the neutral and charged-state absorbances via conjugation (non-limiting examples of substituents are provided in Scheme 1).

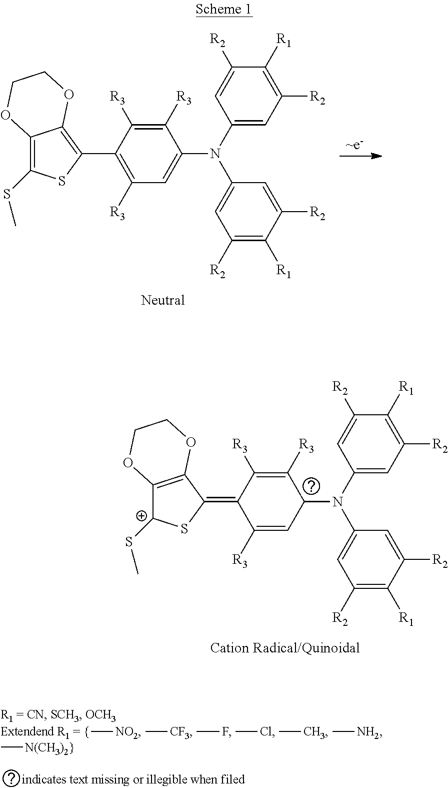

The position R₃ represents the placement of a substituent on the phenyl ring that is adjacently coupled to the EDOT. It is directly conjugated into the neutral state structure, and contributes to the electron richness of the highest occupied molecular orbital (HOMO) in the ground state. Upon oxidation to the cation radical, the molecule enters a quinoidal state, where the R₃ positions are still conjugated into the pi system of the singularly occupied molecular orbital (SOMO). The placement of the R₃ substituents will vary the steric interactions on the EDOT, but also on the freely rotating phenyl pendant rings. R₁ represents the substituents placed on the para position corresponding to the TPA unit. It increases the electron richness of the TPA unit conjugated in the neutral state structure. It also a serves as a stabilizer and prevents follow up reactions upon oxidation, such as polymerization, which may result from large spin densities. Upon further oxidation, the nitrogen on the amine is an additional center for a radical to form. The position labeled as R₂ (meta) will be conjugated into the pi system of the SOMO of the TPA unit.

This approach allows for direct manipulation and evaluation in electron richness of the SOMO in the charged state around the phenyl ring coupled to the EDOT, and free phenyl pendant rings conjugated to the amine center.

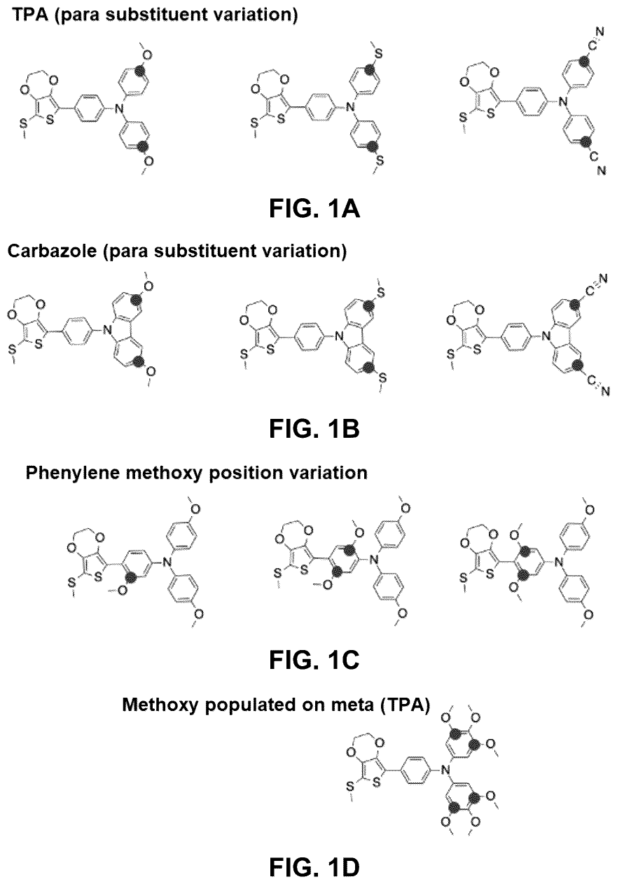

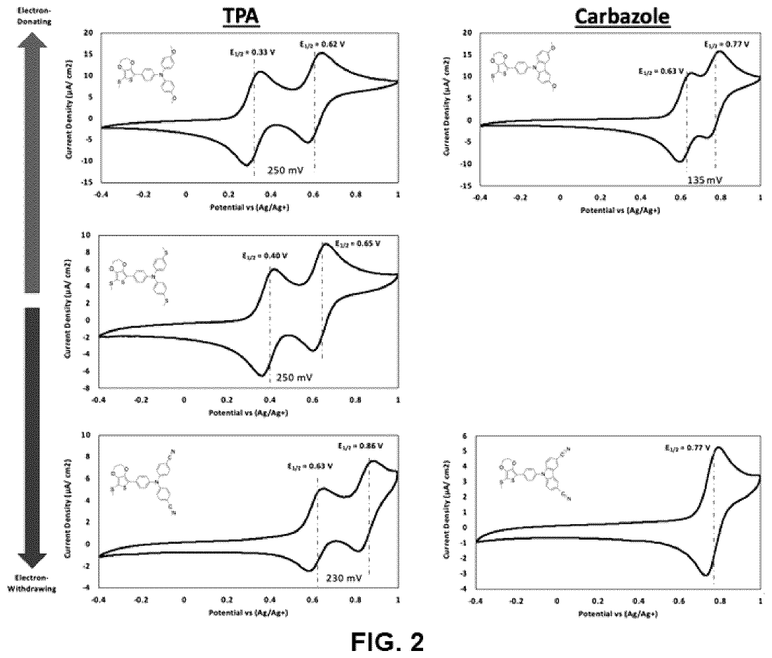

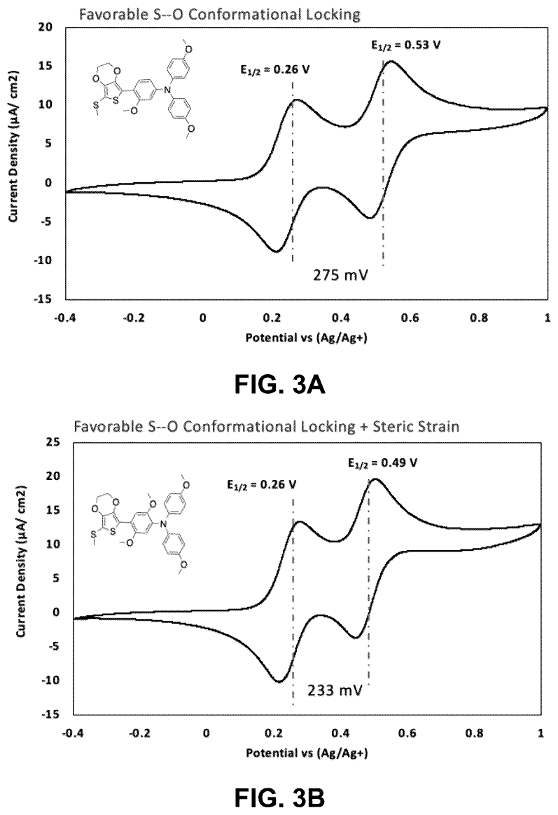

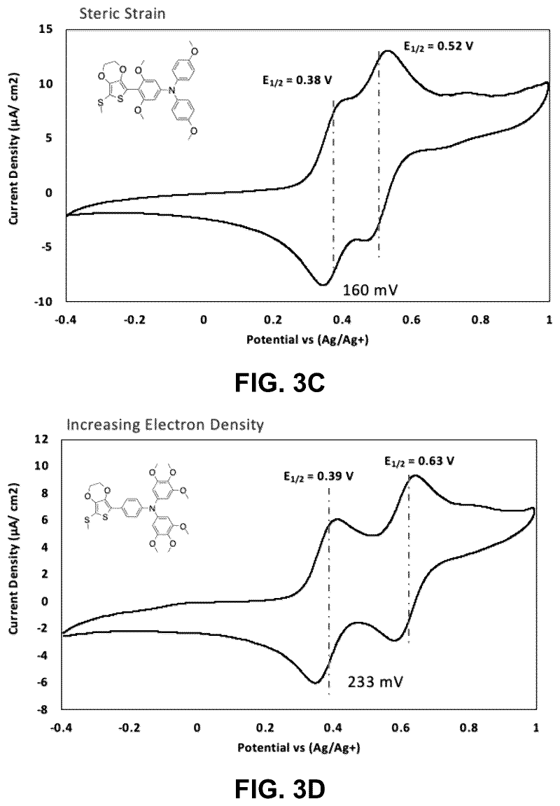

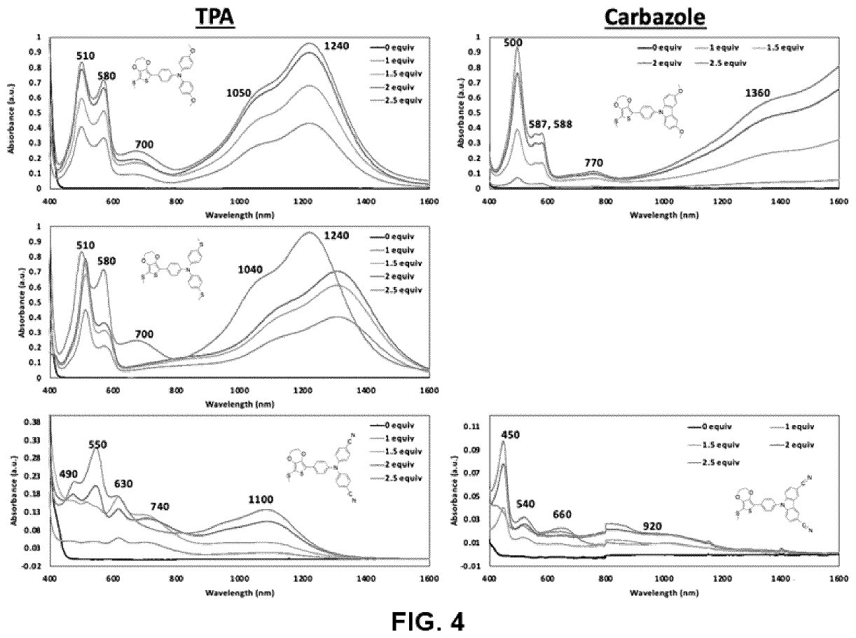

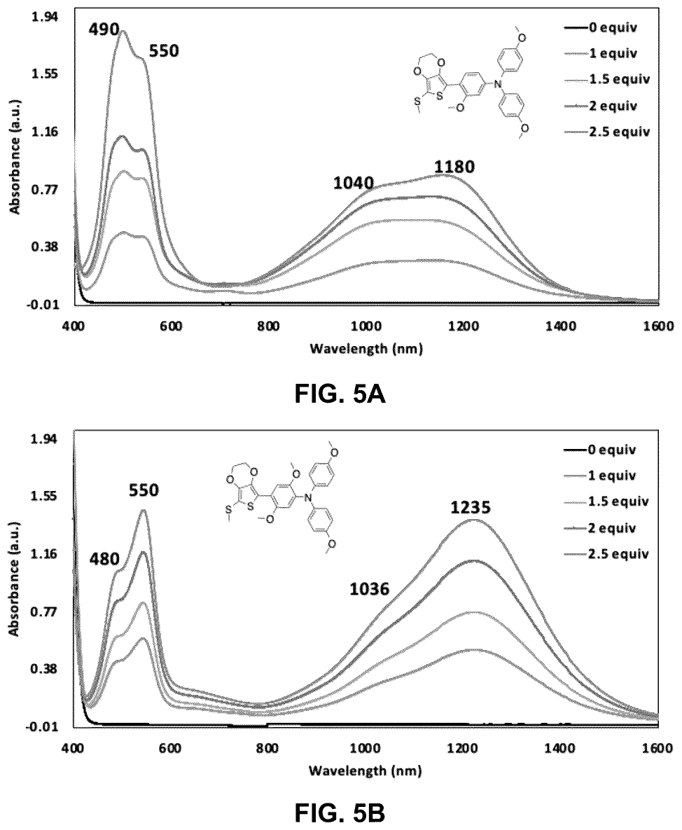

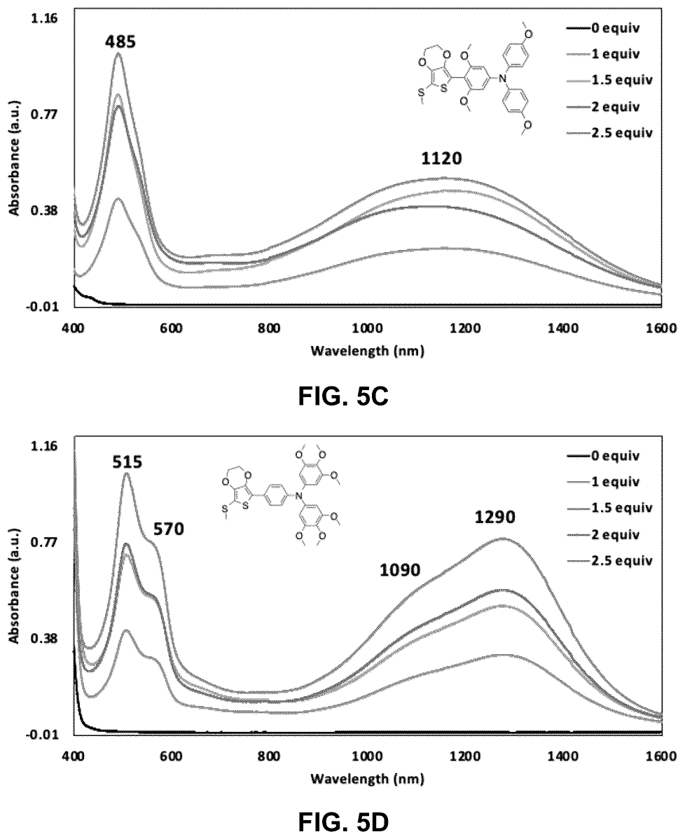

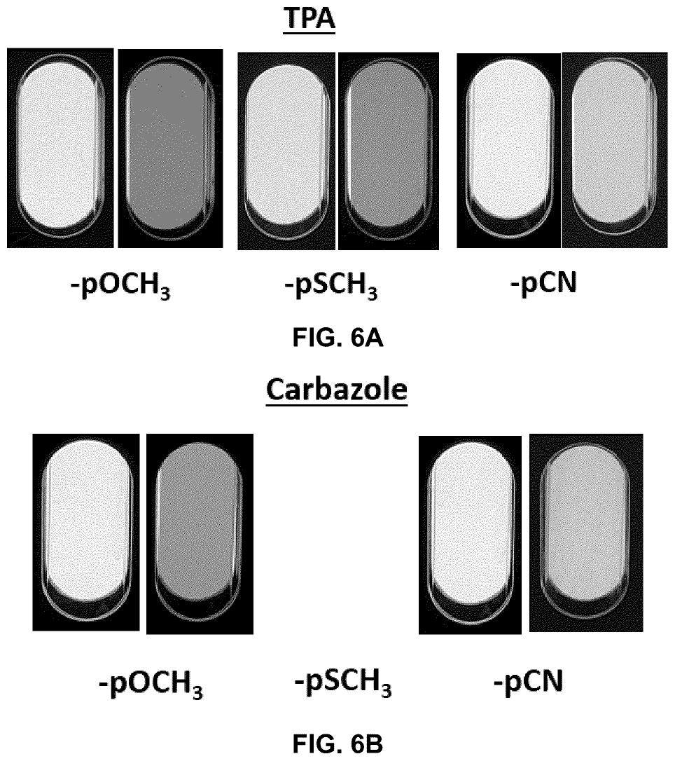

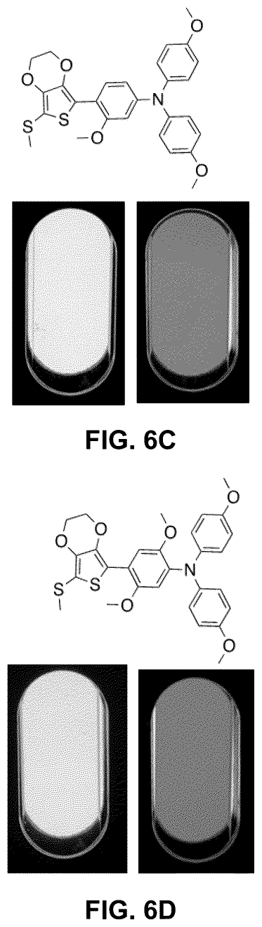

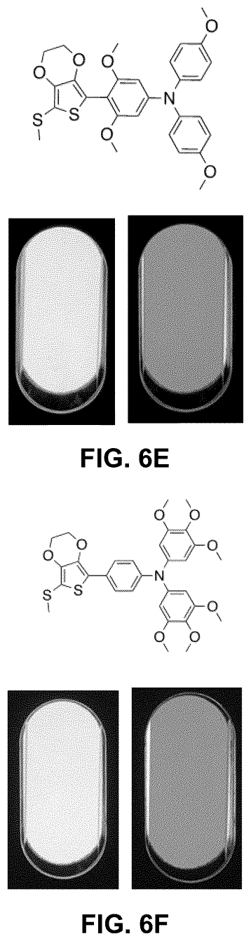

A set of anodically coloring molecules with a new structural motif have been synthesized (FIGS. 1A-1D). The chromophores presented from this experiment show that high contrast and vibrant colors can be reached due to accessing and modulating the absorbance intensity of the radical cation's lower energy (LE) transition peak.

### Example 2: Synthesis and Characterization

### Materials and Methods

All manipulations of oxygen- and moisture-sensitive materials were conducted under a standard Schlenk technique as well as assembled in a glovebox under an argon atmosphere. Analytical thin layer chromatography (TLC) was performed on a plastic support silica gel (0.200 mm thickness) 60 matrix with a fluorescent indicator (silica gel 60F₂₅₄). Standard column chromatography was performed using silica gel, technical grade, 60A, 40-63 μm purchased from Sorbtech.

### General Instrumentation

Proton, carbon, and fluorine nuclear magnetic resonance (¹H, ¹³C, and ¹⁹F) spectra were recorded on a Bruker Avance IIIHD 500 spectrometer (500 MHz ¹H, 126 MHz ¹³C) with solvent resonance as the internal standard. ¹H NMR data are reported as following: chemical shift, multiplicity, J-coupling constants (Hz), and relative integrated intensity. Spectra were processed using MesReNova v14.2. Accurate mass spectra were collected by the Bioanalytical Mass Spectrometry Facility at Georgia Tech on a Micromass Autospec M (EI) Electron spray ionization (ESI) and high-resolution mass spec (HRMS) was taken on Waters Autospec M Three Sector Tandem mass spectrometer. Single-crystal X-ray diffraction (XRD) measurements were performed by mounting single crystals on a loop with paratone on a Synergy-S diffractometer. The crystals were kept at a steady T=100.0 (5) K during data collection. The structure was solved with the ShelXT 2018/2 (Sheldrich, 2018) solution program using dual methods and by using Olex2 1.5-alpha (Dolomanov et al, 2009) as the graphical interface. The model was refined with ShelXL 2018/3 (Sheldrich, 2015) using full matrix least squares minimization on F². Elemental analysis (CHNSF) compositions were sent and performed by Atlantic Microlab. Cyclic Voltammetry analysis was performed using a EG&G Princeton Applied Research 273A potentiostat/galvanostat under Corrware control. Optical absorption spectra and Spectroelectrochemistry were collected at room temperature using an Agilent Cary 5000 UV-vis-NIR scan dual-beam spectrophotometer and Scan software, using either an optically transparent thin layer electrode (OTTLE) cuvette for solution electrochemical experiments or a standard rectangular quartz spectrophotometer cell with a 10 mm pathlength obtained from Starna Cells, Inc for general optical measurements and doping. The current and voltage were controlled and measured a WaveNow portable potentiostat under control of AfterMath software with additional long duration optical measurements carried out using an Ocean Optics USB 2000+ fiber-optic spectrophotometer.

### Reagents and Chemicals

The following reagents were purchased from commercial suppliers and used as received: 3,4-ethylenedioxythiophene (95%, Biosynth International Inc), dimethyl disulfide (>99%, Sigma Aldrich), 2.5 M n-butyllithium in hexanes (Sigma Aldrich), tributyltin chloride (96%, Sigma Aldrich), tetrakis(triphenylphosphine)palladium (0) (99%, Sigma Aldrich), 4-Bromo-4′,4″-dimethoxytriphenylamine (98%, TCI), 4,4′-Dibromotriphenylamine (98%, TCI), m-anisidine (98%, TCI), 3,5-dimethoxyaniline (97%, AmBeed), 1,4-dibromobenzene (98%, Sigma Aldrich), 4-iodoanisole (98%, AmBeed), Potassium Carbonate (99%, VWR), 5-iodo-1,2,3-trimethoxybenzene (95%, aablocks), anhydrous 1,10-phenanthroline (Sigma Aldrich), Cuprous Iodide (99%, Sigma Aldrich), ethylenediamine (99%, Sigma Aldrich), Aniline (99%, Sigma Aldrich), tetrabutylammonium hexafluorophosphate (AmBeed, 98% recrystallized from hot ethanol), Iron (III) Trifluoromethanesulfonate (90%, Sigma Aldrich), Nitrosonium Hexafluorophosphate (95%, Fischer Scientific), N-Bromosuccinimide (99%, Sigma Aldrich), Potassium Tert-Butoxide (97%, ThermoFisher Scientific), Sodium Sulfate (Fischer Scientific), anhydrous tetrahydrofuran (VWR), anhydrous N,N-dimethylformamide (>99.9%, VWR), 1-4 dioxane (99%, VWR), anhydrous dimethyl acetamide (99%, Fischer Scientific), and anhydrous Toluene (VWR).

### General Synthetic Procedure

The synthetic approach to the monomer and trimer synthesis is displayed in Scheme 2, which is outlined below. A previous reported procedure for the lithiation of 3,4-ethylenedioxythiophene and addition of dimethyl disulfide followed by a subsequent lithiation and trans metalation with tributyltin chloride, and finished by Stille coupling was followed in this order to obtain the 2-MeS-EDOT-TPA variants.

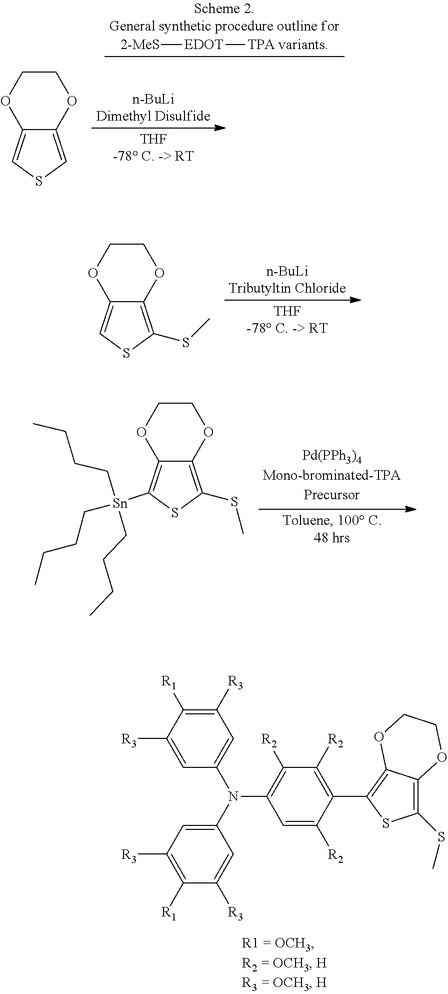

### 2-methylthio-3,4-ethylenedioxythiophene

To an oven dried 50 mL Schlenk tube with a magnetic stir bar was added anhydrous THF (100 mL) and 3,4-ethylenedioxythiophene (10 g, 70.34 mmoles, 1 eq.). The reaction solvent was sparged with argon for 20 minutes followed by 3 consistent cycles of evacuating and refilling the vessel with argon before sealing. The reaction was then cooled to −78° C. before slow dropwise addition of 2.5 M n-butyllithium solution (28.14 mL, 70.34 mmoles, 1 eq.) was carefully added. The reaction was left to stir at −78° C. for 1 full hour before slow addition of dimethyl disulfide (9.98 g, 106 mmoles, 1.5 eq.) was added. The reaction was left to slowly warm from −78° C. to room temperature and stirred overnight. Afterwards, the reaction was quenched with 1 mL of methanol, concentrated to complete dryness, re-dissolved in dichloromethane and washed with distilled water and brine. All organic fractions were collected, dried over with sodium sulfate, filtered, and concentrated in vacuo. The crude product was purified by silica gel column chromatography (1:9) EtOAc:Hexanes to give a clear transparent viscous oil (12.4 g, 94% yield). ¹H NMR (500 MHz, CD₂Cl₂). δ 6.38 (s, 1H), 4.31-4.27 (m, 2H), 4.22-4.19 (m, 2H), 2.37 (s, 3H). ¹³C NMR (126 MHz, CD₂Cl₂) δ 143.42, 141.60, 108.56, 101.57, 65.01, 64.46, 20.91.

### 2-methylthio-3,4-ethylenedioxy-5-tributyltin-thiophene

Into a dry Schlenk flask under an argon atmosphere and with a magnetic stir bar, was added 2-thiomethyl-3,4-ethylenedioxythiophene (1.5 g, 7.97 mmoles, 1 eq.) and anhydrous THF (20 mL). The solution was cooled to −78° C. and 2.5 M n-BuLi solution (3.19 mL, 7.97 mmoles, 1 eq.) was slowly added. After 1 hour, tributyltin chloride (2.54 g, 7.97 mmoles, 1 eq.) was added to the reaction and left to stir to room temperature overnight. The reaction mixture was concentrated in vacuo, redissolved in dichloromethane, and the organic was washed with distilled water and brine. All organic layers were recollected, dried over with sodium sulfate, filtered, and concentrated in vacuo. The resulting yellow viscous oil was used without further purification (3.8 g-quantitative yield, 100%). ¹H NMR (500 MHz, CD₂Cl₂) δ 4.32-4.21 (m, 2H), 4.21-4.05 (m, 2H), 2.37 (s, 3H), 1.60 (ddt, J=9.3, 7.2, 1.7 Hz, 6H), 1.40-1.35 (m, 6H), 1.20-1.09 (m, 6H), 0.93 (t, J=7.4 Hz, 9H). ¹³C NMR (126 MHz, CD₂Cl₂) δ 147.43, 143.68, 113.64, 112.16, 64.97, 64.33, 28.94, 27.18, 20.80, 13.45, 10.60.

### 3-methoxy-N,N-bis(4-methoxyphenyl)aniline

A 250 mL Schlenk flask with a stir bar was charged with 3-methoxyaniline (2.10 g, 17.02 mmoles, 1 eq.), 4-iodoanisole (9.96 g, 42.55 mmoles, 2.5 eq.), 1,10-phenanthroline (613 mg, 3.4 mmoles, 0.2 eq.), Copper (I) Iodide (648 mg, 3.404 mmoles, 0.2 eq), and potassium tert-butoxide (15.3 g, 136.16 mmoles, 8 eq.). Then 150 mL of Toluene was added and sparged with argon for 20 minutes followed by 3 consistent cycles of evacuating and refilling the vessel with argon before sealing. The mixture was heated to 120° C. and stirred overnight. After cooling to room temperature, the mixture was concentrated to complete dryness, and redissolved in DCM. The organic phase was washed with distilled water (3×) and brine (1×). All organic layers were collected, dried over with sodium sulfate, filtered, and concentrated to complete dryness. The crude product was purified by silica gel chromatography (1:1) hexanes:DCM to obtain a red viscous oil (3.31 g, 58% yield). ¹H NMR (500 MHz, CD₂Cl₂) δ 7.07 (d, J=9.0 Hz, 5H), 6.87 (d, J=9.0 Hz, 4H), 6.46 (dd, J=2.4, 1.5 Hz, 3H), 3.82 (s, 6H), 3.71 (s, 3H). ¹³C NMR (126 MHz, CD₂Cl₂) δ 160.43, 156.04, 150.21, 140.87, 129.46, 126.74, 114.57, 112.98, 106.37, 105.46, 55.41, 55.03.

### 4-bromo-3-methoxy-N,N-bis(4-methoxyphenyl)aniline

A 100 mL round bottom flask was charged with 3-methoxy-N,N-bis(4-methoxyphenyl)aniline (1 g, 2.98 mmoles, 1 eq.), N-bromosuccinimide (0.531 g, 2.98, 1.05 mmoles) and DCM (100 mL). The reaction was wrapped in aluminum foil and stirred overnight at room temperature. Afterwards, the solution was washed with distilled water (×3), and brine (1×). All organic phases were recollected, dried over with sodium sulfate, filtered, and concentrated in vacuo. The crude product was purified by silica gel chromatography using (1:1) Hexanes:DCM as the eluant. The product obtained was a green viscous oil (0.758 grams, 62% yield). ¹H NMR (500 MHz, CD₂Cl₂) δ 7.27 (d, J=8.6 Hz, 1H), 7.13-7.03 (m, 4H), 6.91-6.84 (m, 4H), 6.53 (d, J=2.5 Hz, 1H), 6.37 (dd, J=8.7, 2.6 Hz, 1H), 3.82 (s, 6H), 3.69 (s, 3H). ¹³C NMR (126 MHz, CD₂Cl₂) δ 156.28, 140.41, 132.77, 126.80, 114.66, 113.53, 104.50, 55.91, 55.41.

### 2,5-dimethoxy-N,N-bis(4-methoxyphenyl)aniline

A 250 mL Schlenk flask with a stir bar was charged with 2,5-dimethoxyaniline (2.0 g, 13.06 mmoles, 1 eq.), 4-iodoanisole (7.64 g, 32.65 mmoles, 2.5 eq.), 1,10-phenanthroline (471 mg, 2.61 mmoles, 0.2 eq.), Copper (I) Iodide (497 mg, 2.61 mmoles, 0.2 eq), and potassium tert-butoxide (11.72 g, 104.48 mmoles, 8 eq.). Then 150 mL of toluene was added and sparged with argon for 20 minutes followed by 3 consistent cycles of evacuating and refilling the vessel with argon before sealing. The mixture was heated to 120° C. and stirred overnight. After cooling to room temperature, the mixture was concentrated to complete dryness, and redissolved in DCM. The organic phase was washed with distilled water (3×) and brine (1×). All organic layers were collected, dried over with sodium sulfate, filtered, and concentrated to complete dryness. The crude product was purified by silica gel chromatography (1:1) hexanes:DCM to obtain a red viscous oil (1.94 g, 41% yield). ¹H NMR (500 MHz, CD₂Cl₂) δ 6.90 (d, J=9.1 Hz, 4H), 6.80 (d, J=9.1 Hz, 4H), 6.70 (dd, J=8.9, 3.1 Hz, 1H), 6.65 (d, J=3.1 Hz, 1H), 3.79 (s, 6H), 3.71 (s, 3H), 3.62 (s, 3H). ¹³C NMR (126 MHz, CD₂Cl₂) δ 154.72, 154.56, 149.64, 141.74, 137.56, 123.13, 114.92, 114.73, 114.14, 109.85, 56.67, 55.51, 55.41.

### 4-Bromo-2,5-dimethoxy-N,N-bis(4-methoxyphenyl)aniline

A 100 mL round bottom flask was charged with 2,5-dimethoxy-N,N-bis(4-methoxyphenyl)aniline (1 g, 2.74 mmoles, 1 eq.), N-bromosuccinimide (0.487 g, 2.74, 1.05 mmoles) and DCM (100 mL). The reaction was wrapped in aluminum foil and stirred overnight at room temperature. Afterwards, the solution was washed with distilled water (×3), and brine (1×). All organic phases were recollected, dried over with sodium sulfate, filtered, and concentrated in vacuo. The crude product was purified by silica gel chromatography using (3:1) Hexanes:DCM as the eluant. The product obtained was a red viscous oil (0.617 grams, 51% yield). ¹H NMR (500 MHz, CD₂Cl₂) δ 7.17 (s, 1H), 6.98-6.86 (m, 4H), 6.86-6.73 (m, 4H), 6.70 (s, 1H), 3.79 (s, 6H), 3.71 (s, 3H), 3.61 (s, 3H). ¹³C NMR (126 MHz, CD₂Cl₂) δ 154.88, 150.87, 149.72, 141.33, 136.78, 123.16, 119.07, 114.19, 112.84, 106.37, 56.86, 56.70, 55.40.

### 3,5-dimethoxy-N,N-bis(4-methoxyphenyl)aniline

A 250 mL Schlenk flask with a stir bar was charged with 3,5-dimethoxyaniline (2 g, 13.06 mmoles, 1 eq.), 4-iodoanisole (7.64 g, 32.64 mmoles, 2.5 eq.), 1,10-phenanthroline (471 mg, 2.61 mmoles, 0.2 eq.), Copper (1) Iodide (497 mg, 2.612 mmoles, 0.2 eq), and potassium tert-butoxide (11.73 g, 104.5 mmoles, 8 eq.). Then 150 mL of Toluene was added and sparged with argon for 20 minutes followed by 3 consistent cycles of evacuating and refilling the vessel with argon before sealing. The mixture was heated to 120° C. and stirred overnight. After cooling to room temperature, the mixture was concentrated to complete dryness, and redissolved in DCM. The organic phase was washed with distilled water (3×) and brine (1×). All organic layers were collected, dried over with sodium sulfate, filtered, and concentrated to complete dryness. The crude product was purified by silica gel chromatography (1:1) hexanes:DCM to obtain a red viscous oil (3.76 g, 79% yield). ¹H NMR (400 MHz, CD₂Cl₂) δ 7.08 (d, J=8.9 Hz, 4H), 6.86 (d, J=8.9 Hz, 4H), 6.13-5.94 (m, 3H), 3.81 (d, J=1.0 Hz, 6H), 3.68 (d, J=0.9 Hz, 6H). ¹³C NMR (101 MHz, CD₂Cl₂) δ 161.24, 156.15, 150.77, 140.70, 126.99, 114.53, 98.79, 92.30, 55.40, 55.10.

### 4-bromo-3,5-dimethoxy-N,N-bis(4-methoxyphenyl)aniline

A 100 mL round bottom flask was charged with 3,5-dimethoxy-N,N-bis(4-methoxyphenyl)aniline (1 g, 2.74 mmoles, 1 eq.), N-bromosuccinimide (0.487 g, 2.74, 1.05 mmoles) and DCM (100 mL). The reaction was wrapped in aluminum foil and stirred overnight at room temperature. Afterwards, the solution was washed with distilled water (×3), and brine (1×). All organic phases were recollected, dried over with sodium sulfate, filtered, and concentrated in vacuo. The crude product was purified by silica gel chromatography using (1:1) Hexanes:DCM as the eluant. The product obtained was a yellow viscous oil (0.989 grams, 81% yield). ¹H NMR (400 MHz, CD₂Cl₂) δ 7.09 (d, J=8.9 Hz, 4H), 6.88 (d, J=8.9 Hz, 4H), 6.16 (s, 2H), 3.82 (s, 6H), 3.68 (s, 6H). ¹³C NMR (101 MHz, CD₂Cl₂) δ 157.12, 156.29, 149.64, 140.36, 126.92, 114.62, 97.52, 56.10, 55.40.

### 3,4,5-trimethoxy-N-phenyl-N-(3,4,5-trimethoxyphenyl)aniline

A 150 mL Schlenk flask with a stir bar was charged with aniline (380 mg, 4.08 mmoles, 1 eq.), 5-iodo-1,2,3-trimethoxybenzene (3.00 g, 10.2 mmoles, 2.5 eq.), 1,10-phenanthroline (147 mg, 0.82 mmoles, 0.2 eq.), Copper (1) Iodide (155 mg, 0.82 mmoles, 0.2 eq), and potassium tert-butoxide (3.66 g, 32.64 mmoles, 8 eq.). Then 50 mL of toluene was added and sparged with argon for 20 minutes followed by 3 consistent cycles of evacuating and refilling the vessel with argon before sealing. The mixture was heated to 120° C. and stirred overnight. After cooling to room temperature, the mixture was concentrated to complete dryness, and redissolved in DCM. The organic phase was washed with distilled water (3×) and brine (1×). All organic layers were collected, dried over with sodium sulfate, filtered, and concentrated to complete dryness. The crude product was purified by silica gel chromatography 100% DCM to obtain a red oil (0.981 g, 57% yield). ¹H NMR (500 MHz, CDCl₃) δ 7.28-7.20 (m, 2H), 7.14-7.05 (m, 2H), 7.05-6.92 (m, 1H), 6.33 (s, 4H), 3.87 (s, 6H), 3.73 (s, 12H). ¹³C NMR (126 MHz, CDCl₃) δ 153.60, 147.75, 143.66, 134.10, 129.07, 123.40, 122.28, 102.17, 61.03, 56.16.

### N-(4-bromophenyl)-3,4,5-trimethoxy-N-(3,4,5-trimethoxyphenyl)aniline

A 100 mL round bottom flask was charged with 3,4,5-trimethoxy-N-phenyl-N-(3,4,5-trimethoxyphenyl)aniline (0.981 g, 2.31 mmoles, 1 eq.), N-bromosuccinimide (0.410 g, 2.31, 1.05 mmoles) and DCM (50 mL). The reaction was wrapped in aluminum foil and stirred overnight at room temperature. Afterwards, the solution was washed with distilled water (×3), and brine (1×). All organic phases were recollected, dried over with sodium sulfate, filtered, and concentrated in vacuo. The crude product was purified by silica gel chromatography using a gradient of 100% DCM to 100% EtOAc as the eluant. The product obtained was a dark red viscous oil (0.835 grams, 71% yield). ¹H NMR (500 MHz, CDCl₃) δ 7.38-7.24 (m, 2H), 6.94 (d, J=8.9 Hz, 2H), 6.30 (s, 4H), 3.86 (s, 6H), 3.73 (s, 12H). ¹³C NMR (126 MHz, CDCl₃) δ 153.70, 146.95, 143.11, 134.45, 131.98, 124.46, 114.30, 102.32, 61.01, 56.19.

### General Stille Coupling Procedure

To a 3-neck round bottom flask was added 2-methylthio-3,4-ethylenedioxy-5-tributyltin-thiophene (0.5 g, 1.05 mmoles, 1 eq.), aryl bromide (1.05 mmoles, 1 eq.), and Pd(PPh₃)₄ (0.083 mmoles, 0.08 eq.), and anhydrous toluene in a glovebox. The reaction was sealed but the solution was further sparged with argon for 20 minutes, before stirring at 100° C. for 48 hours. Afterwards, the reaction was left to cool down, rotary evaporated to complete dryness, redissolved in dichloromethane, and the organic solution was washed with distilled water and brine. All the organic fractions were recollected, dried over with sodium sulfate, filtered, and concentrated in vacuo. The resulting 2-MeS-EDOT TPA variants were purified by column chromatography using a gradient of 100% hexanes, 50% hexanes:dichloromethane, to 100% dichloromethane as the eluting solvent.

### 4-methoxy-N-(4-methoxyphenyl)-N-(4-(7-(methylthio)-2,3-dihydrothieno[3,4-b][1,4]dioxin-5-yl)phenyl)aniline [EDOT-TPA-pOMe₂]

Compound was obtained as a pale yellow powder, 295 mg, 63% yield. Melting Point: 183° C. ¹H NMR (400 MHz, CD₂Cl₂) δ 7.57-7.36 (m, 2H), 7.21-7.02 (m, 4H), 6.99-6.72 (m, 6H), 4.39-4.19 (m, 4H), 3.82 (s, 6H), 2.39 (s, 3H). ¹³C NMR (101 MHz, CD₂Cl₂) δ 156.14, 140.58, 126.73, 126.66, 120.09, 114.64, 64.88, 64.50, 55.42. Anal. Calcd for C₂₇H₂₈NO₄S₂: C, 65.96; H, 5.13; S, 13.04; N, 2.85. Found: C, 63.57; H, 5.18; S, 13.04; N, 2.46.

### 3-methoxy-N,N-bis(4-methoxyphenyl)-4-(7-methylthio)-2,3-dihydrothieno[3,4-b][1,4]dioxin-5-yl)aniline [EDOT-2-methoxy-TPA-pOMe₂]

Compound was obtained as a golden yellow powder; 350 mg, 64% yield. Melting Point: 123° C. ¹H NMR (500 MHz, CD₂Cl₂) δ 7.70 (dd, J=8.7, 0.9 Hz, 1H), 7.11 (dd, J=9.0, 0.9 Hz, 4H), 6.88 (dd, J=9.0, 0.9 Hz, 4H), 6.61-6.31 (m, 2H), 4.44-4.13 (m, 4H), 3.86-3.78 (m, 6H), 3.71 (d, J=0.9 Hz, 3H), 2.39 (d, J=0.9 Hz, 3H). ¹³C NMR (126 MHz, CD₂Cl₂) δ 156.23, 140.44, 129.63, 126.90, 114.61, 112.07, 103.32, 77.03, 64.79, 64.46, 55.41, 55.36. Anal. Calcd for C₂₈H₂₇NO₅S₂: C, 64.47; H, 5.22; S, 12.29; N, 2.69. Found: C, 62.57; H, 5.63; S, 11.64; N, 2.41.

### 2,5-dimethoxy-N,N-bis(4-methoxyphenyl)-4-(7-methylthio)-2,3-dihydrothieno[3,4-b][1,4]dioxin-5-yl)aniline [EDOT-2,5-dimethoxy-TPA-POMe₂]

Compound was obtained as a green powder, 251 mg, 43% yield. Melting Point: 134° C. ¹H NMR (500 MHz, CD₂Cl₂) δ 7.14 (d, J=9.0 Hz, 4H), 6.89 (d, J=9.0 Hz, 4H), 6.13 (s, 2H), 4.32-4.25 (m, 2H), 4.21-4.15 (m, 2H), 3.83 (s, 6H), 3.59 (s, 6H), 2.41 (s, 3H). ¹³C NMR (126 MHz, CD₂Cl₂) δ 159.07, 156.41, 150.97, 142.99, 140.25, 127.31, 114.61, 96.03, 64.92, 64.32, 55.71, 55.40. Anal. Calcd for C₂₉H₂₉NO₆S₂: C, 63.14; H, 5.30; S, 11.62; N, 2.54. Found: C, 62.57; H, 5.55; S, 10.34; N, 2.44.

### 3,5-dimethoxy-N,N-bis(4-methoxyphenyl)-4-(7-methylthio)-2,3-dihydrothieno[3,4-b][1,4]dioxin-5-yl)aniline [EDOT-2,6-dimethoxy-TPA-pOMe₂]

Compound was obtained as a dark yellow crystalline powder; 307 mg, 53% yield. Melting Point: 147° C. ¹H NMR (500 MHz, CD₂Cl₂) δ 7.68 (s, 1H), 6.96-6.91 (m, 4H), 6.82-6.78 (m, 4H), 6.69 (s, 1H), 4.37-4.29 (m, 4H), 3.80 (s, 6H), 3.73 (s, 3H), 3.63 (s, 3H), 2.41 (s, 3H). ¹³C NMR (126 MHz, CD₂Cl₂) δ 154.84, 150.80, 148.91, 141.49, 136.05, 123.28, 118.10, 115.37, 114.15, 112.12, 64.74, 64.65, 56.82, 56.27, 55.41. Anal. Calcd for C₂₉H₂₉NO₆S₂: C, 63.14; H, 5.30; S, 11.62; N, 2.54. Found: C, 61.03; H, 5.61; S, 10.37; N, 2.31.

### 3,4,5-trimethoxy-N-(4-(7-(methylthio)2,3-dihydrothieno[3,4-b][1,4]dioxin-5-yl)phenyl)-N-(3,4,5-trimethoxyphenyl)aniline [EDOT-TPA-trimethoxy]

Compound was obtained as a red viscous oil; 286 mg, 48% yield. Melting Point: 70° C. ¹H NMR (400 MHz, CD₂Cl₂) δ 7.57 (d, J=8.8 Hz, 2H), 7.04 (d, J=8.7 Hz, 2H), 6.38 (s, 4H), 4.32 (td, J=5.6, 4.1 Hz, 4H), 3.80 (s, 6H), 3.73 (s, 12H), 2.40 (s, 3H). ¹³C NMR (101 MHz, CD₂Cl₂) δ 159.00, 153.77, 143.14, 126.62, 122.36, 102.67, 64.54, 60.52, 56.04. Anal. Calcd for C₃₁H₃₃NO₈S₂: C, 60.87; H, 5.44; S, 10.48; N, 2.29. Found: C, 61.13; H, 5.52; S, 10.23; N, 2.36.

### Single-Crystal X-Ray Crystallography

2-Mes-EDOT-TPA-pOCH₃: All reflection intensities were measured at 110.00(10) K using a Rigaku XtaLAB Synergy R (equipped with a rotating-anode X-ray source and HyPix-6000HE detector) with Cu Kα radiation (λ=1.54178 Å) under the program CrysAlisPro (Version CrysAlisPro 1.171.42.49, Rigaku OD, 2022). The same program was used to refine the cell dimensions and for data reduction. The structure was solved with the program SHELXT-2018/2 and was refined on F2 with SHELXL-2019/3. Analytical numeric absorption correction using a multifaceted crystal model was applied using CrysAlisPro. The temperature of the data collection was controlled using the system Cryostream 1000 from Oxford Cryosystems. The H atoms were placed at calculated positions using the instructions AFIX 23, AFIX 43 or AFIX 137 with isotropic displacement parameters having values 1.2 U_{eq} of the attached C atoms. The structure is mostly ordered. The moiety —O1-C5-C6-O2- is disordered over two orientations, and the occupancy factor of the major component of the disorder refines to 0.922(4).

2-Mes-EDOT-2,5-dimethoxy-TPA-pOCH₃: All reflection intensities were measured at 110.00(10) K using a Rigaku XtaLAB Synergy R (equipped with a rotating-anode X-ray source and HyPix-6000H E detector) with Cu Kα radiation (λ=1.54178 Å) under the program CrysAlisPro (Version CrysAlisPro 1.171.42.49). The same program was used to refine the cell dimensions and for data reduction. The structure was solved with the program SHELXT-2018/2 (Sheldrick, 2018) and was refined on F2 with SHELXL-2019/3. Analytical numeric absorption correction using a multifaceted crystal model was applied using CrysAlisPro. The temperature of the data collection was controlled using the system Cryostream 1000 from Oxford Cryosystems. The H atoms were placed at calculated positions using the instructions AFIX 23, AFIX 43 or AFIX 137 with isotropic displacement parameters having values 1.2 Ueq of the attached C atoms.

The asymmetric unit contains two crystallographically independent molecules in the asymmetric unit. The structure is mostly ordered. The moiety —O1B—C5B—C6B—O2B— is disordered over two orientations, and the occupancy factor of the major component of the disorder refines to 0.604(8). The absolute configuration has been established by anomalous-dispersion effects in diffraction measurements on the crystal, and the Flack parameter refines to 0.001(3).

2-Mes-EDOT-2,6-dimethoxy-TPA-pOCH₃: All reflection intensities were measured at 110.00(10) K using a Rigaku XtaLAB Synergy R (equipped with a rotating-anode X-ray source and HyPix-6000H E detector) with Cu Kα radiation (λ=1.54178 Å) under the program CrysAlisPro (Version CrysAlisPro 1.171.42.49). The same program was used to refine the cell dimensions and for data reduction. The structure was solved with the program SHELXT-2018/2 and was refined on F2 with SHELXL-2019/3 (Sheldrick, 2018). Analytical numeric absorption correction using a multifaceted crystal model was applied using CrysAlisPro. The temperature of the data collection was controlled using the system Cryostream 1000 from Oxford Cryosystems. The H atoms were placed at calculated positions using the instructions AFIX 23, AFIX 43 or AFIX 137 with isotropic displacement parameters having values 1.2 or 1.5 Ueq of the attached C atoms.

The structure is mostly ordered. The asymmetric unit contains one partially occupied lattice DCM solvent molecule (occupancy factor: 0.292(3)) that is found at one site of threefold axial symmetry (and thus must be disordered). The absolute configuration has been established by anomalous-dispersion effects in diffraction measurements on the crystal, and the Flack and Hooft parameters refine to 0.019(9) and 0.012(3), respectively.

### Example 3: Modulation of Electron Density Around Phenylene Rings Contributes to Oxidation Shift

Numerous resonance structures of TPA-based cation radicals can be generated around the phenyl pendants, as shown in one non-limiting example in Scheme 3 below:

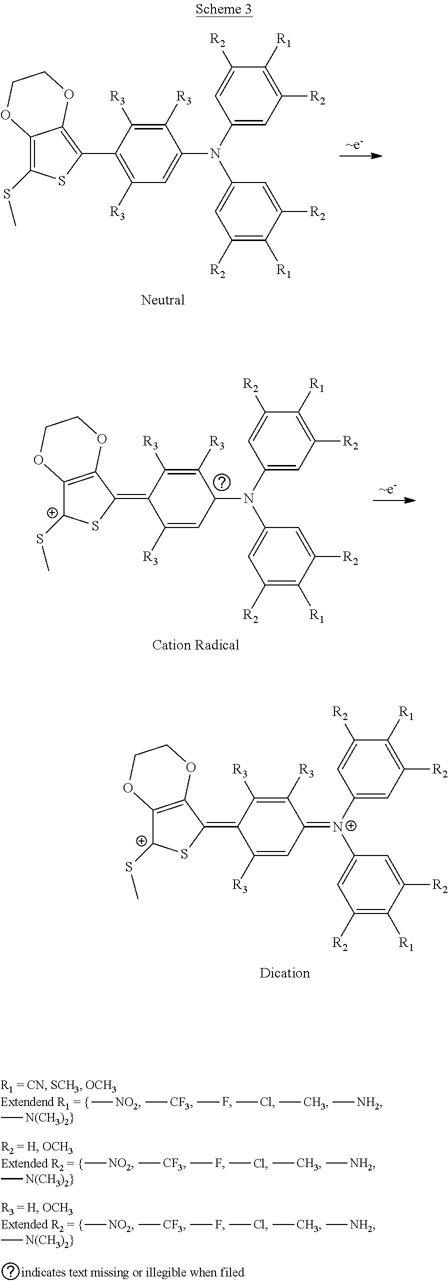

Position of electron density and steric interactions can modulate the oxidation potential peak for both the first onset and second onset as seen in Table 1 below:

TABLE 1
Oxidation Potential, First and Second Onset

| Molecule | E_{ox, 1} (mV) | E_{ox, 2} (mV) |
|---|---|---|
| EDOT-TPA-pOCH₃ | 415 | 707 |
| EDOT-2-(OCH₃)-TPA-pOCH₃ | 239 | 521 |
| EDOT-2,5-(OCH₃)-TPA-pOCH₃ | 251 | 479 |
| EDOT-2,6-(OCH₃)-TPA-pOCH₃ | 377 | 497 |
| EDOT-TPA-3,4,5-trimethoxy | 387 | 607 |

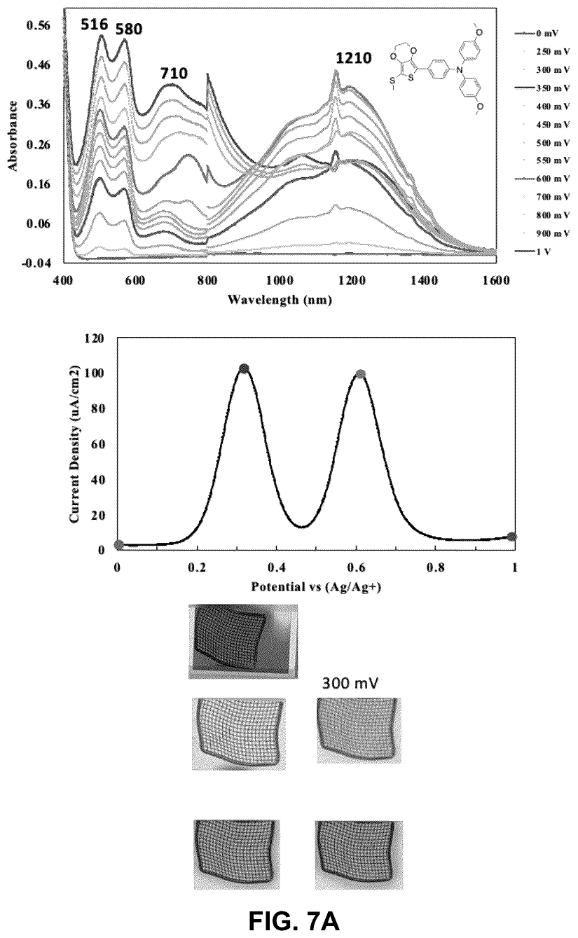

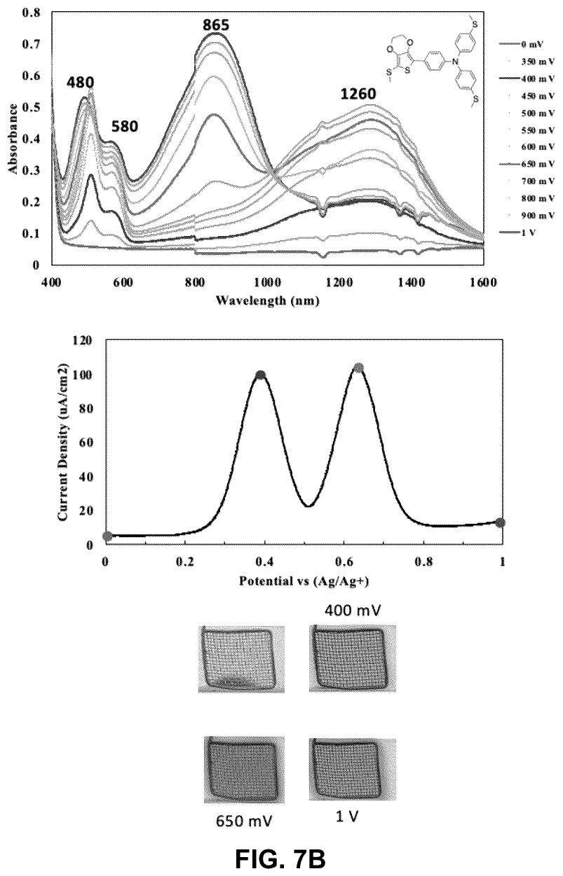

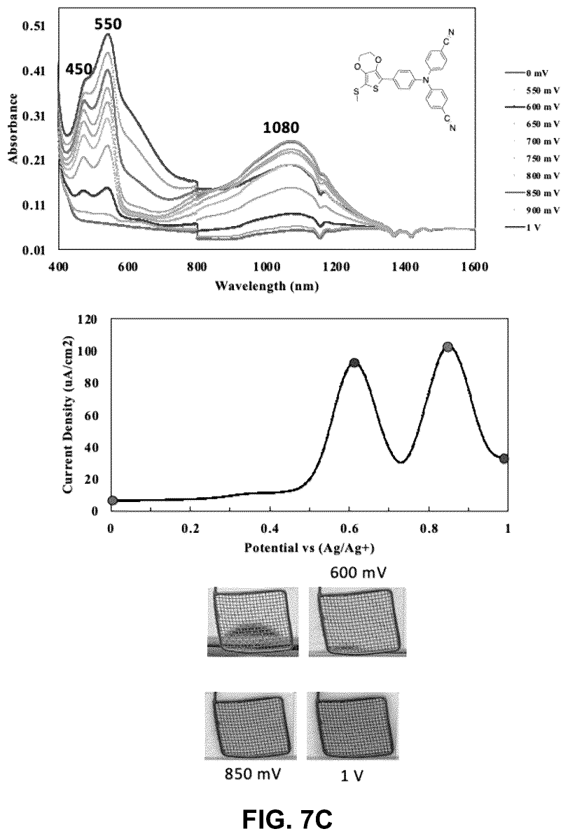

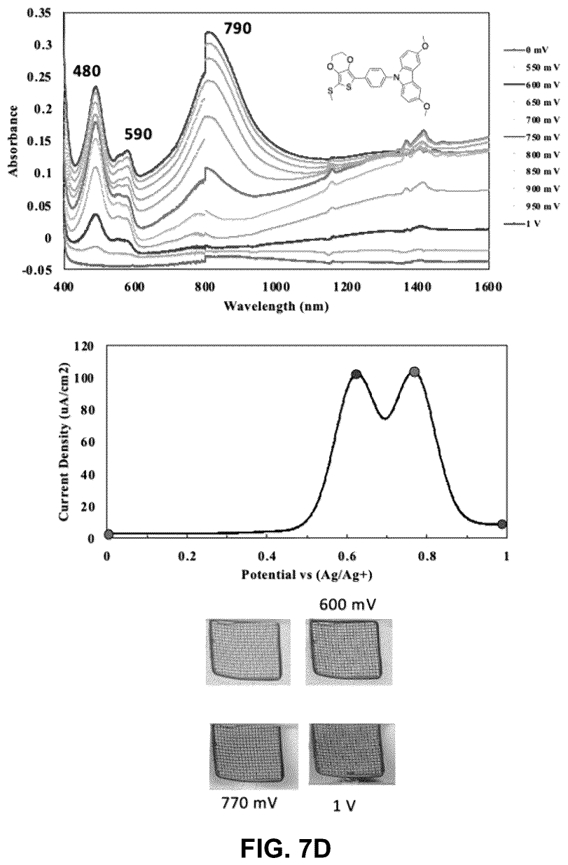

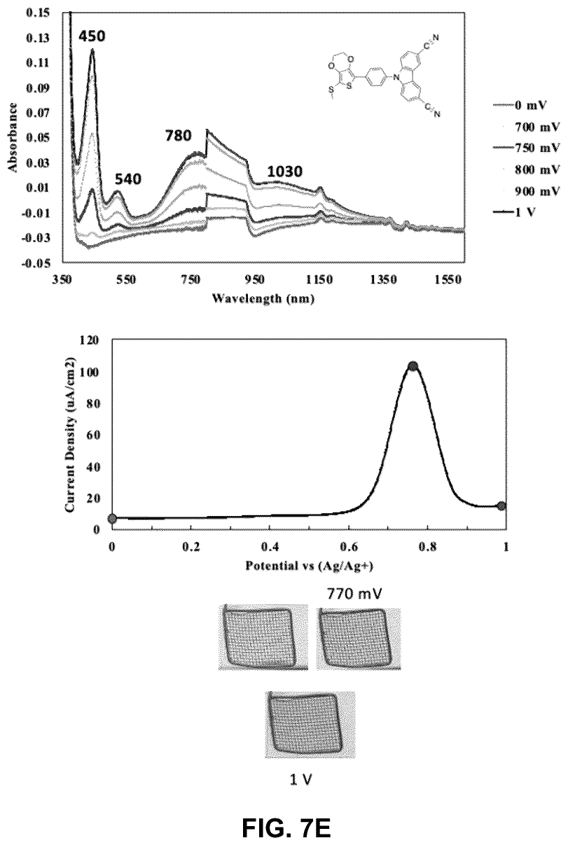

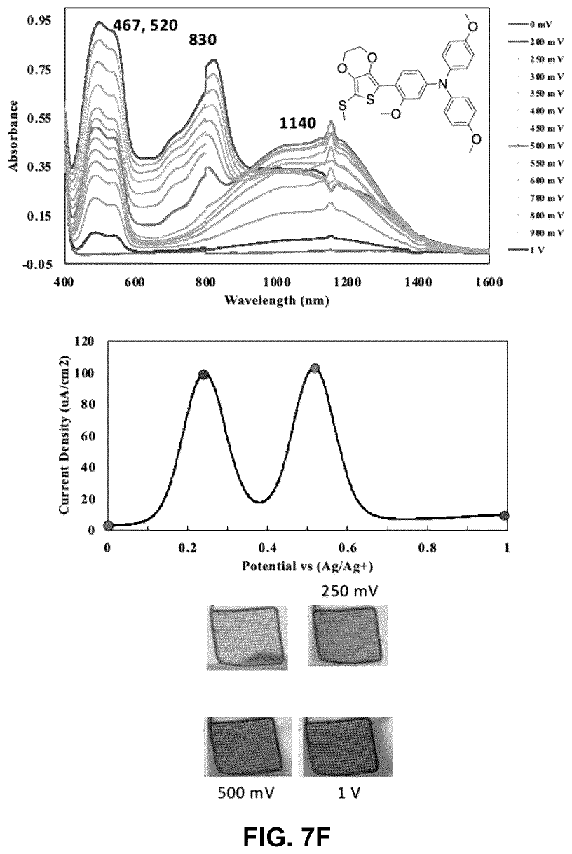

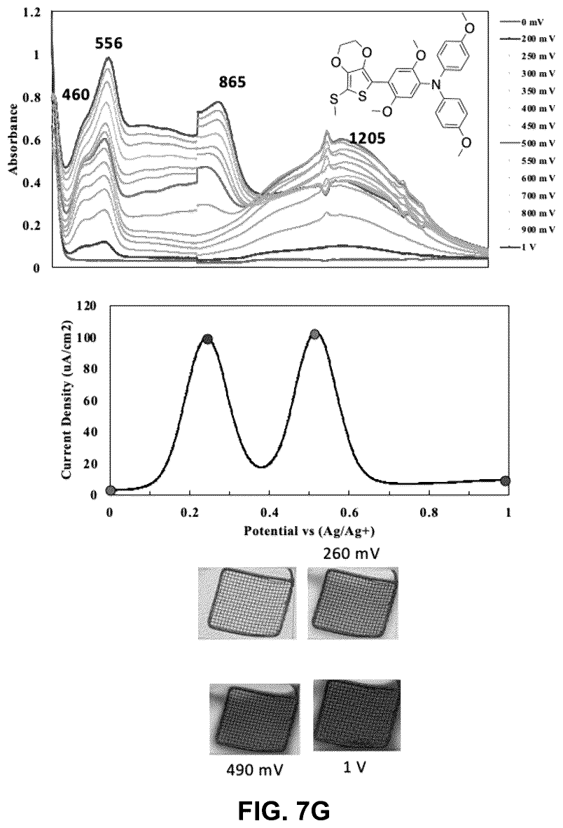

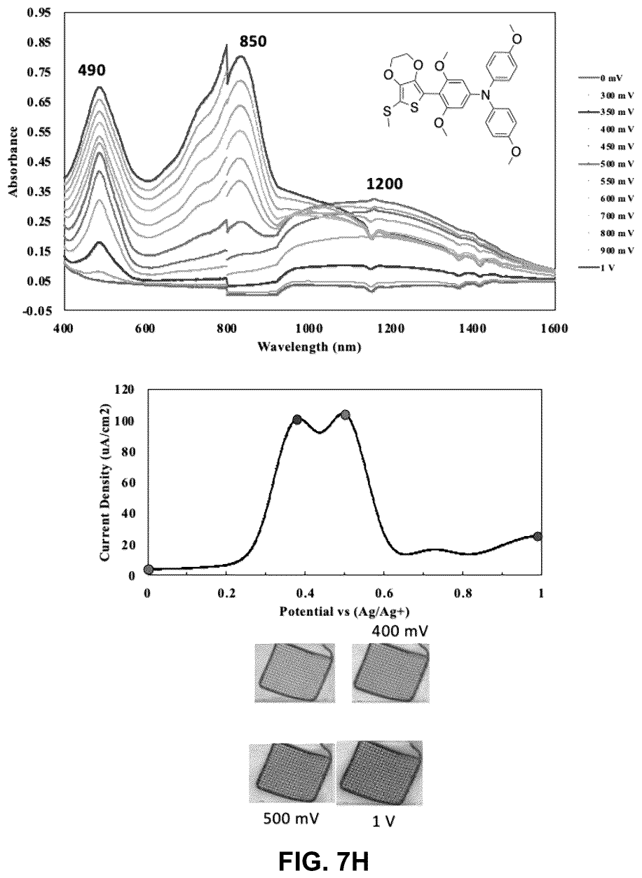

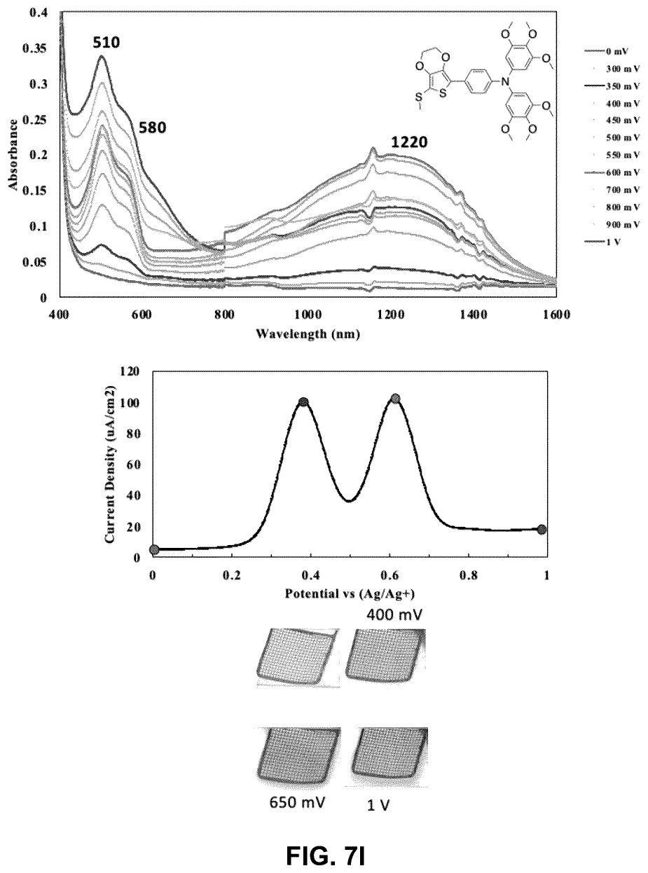

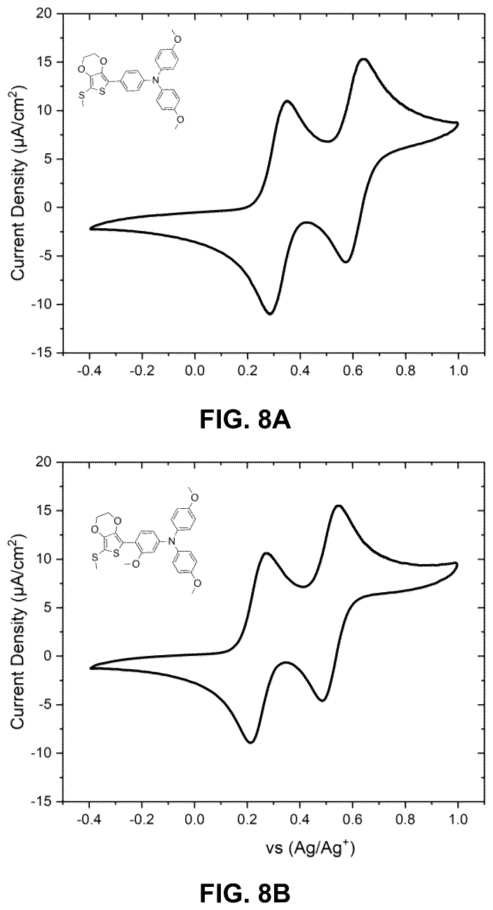

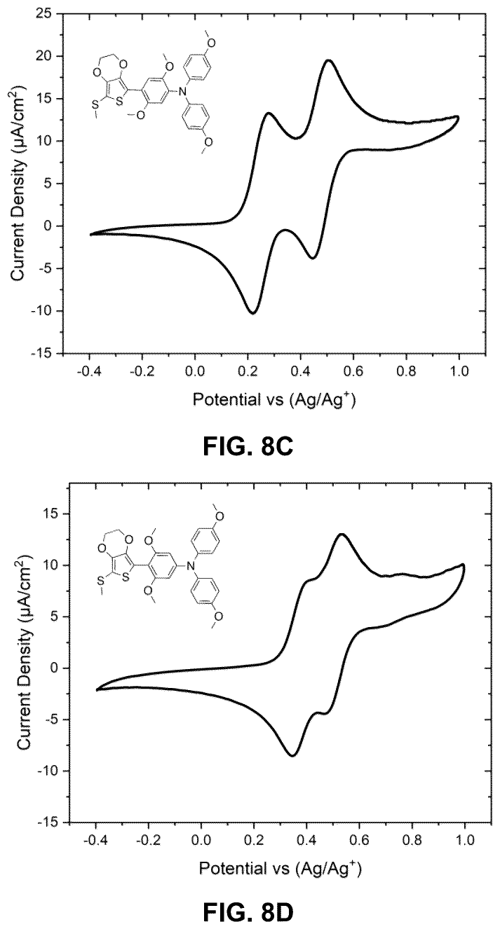

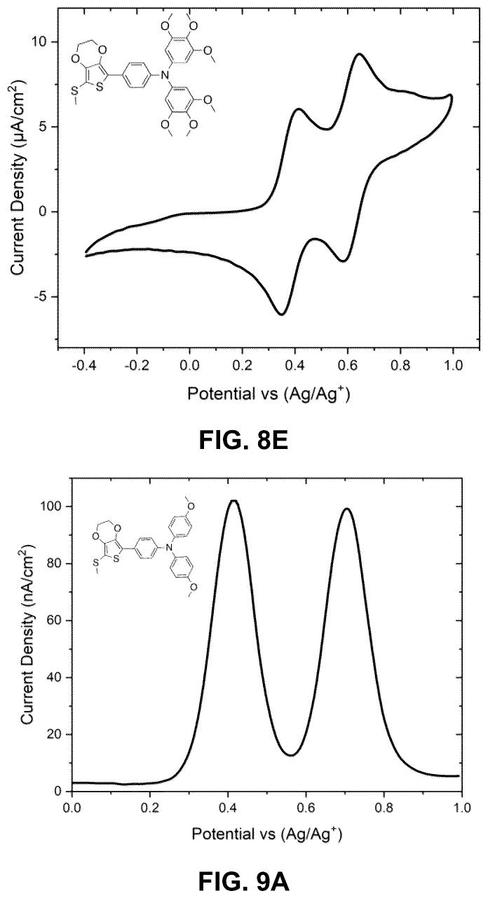

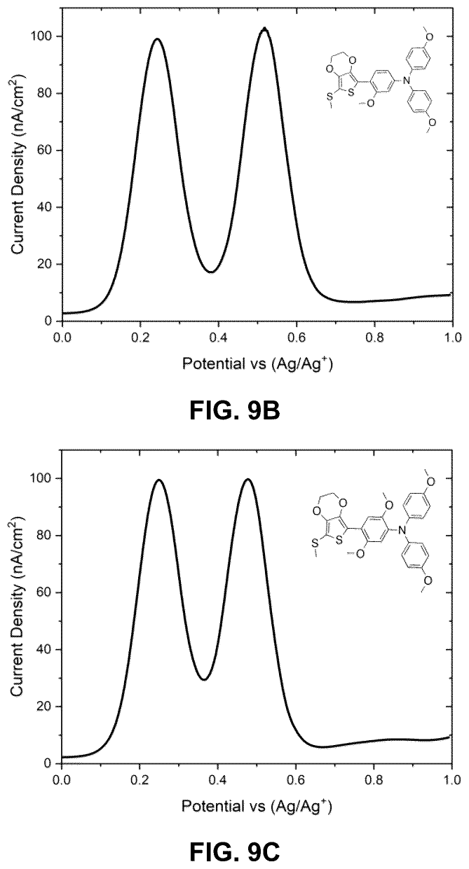

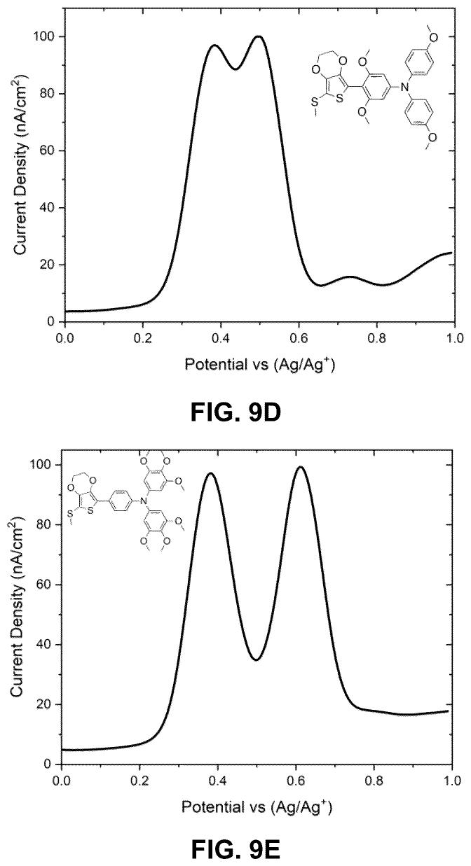

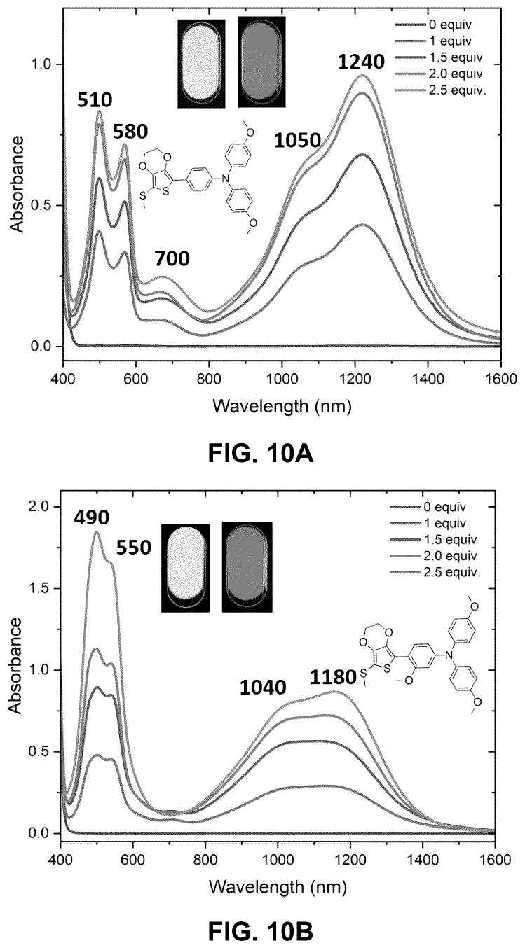

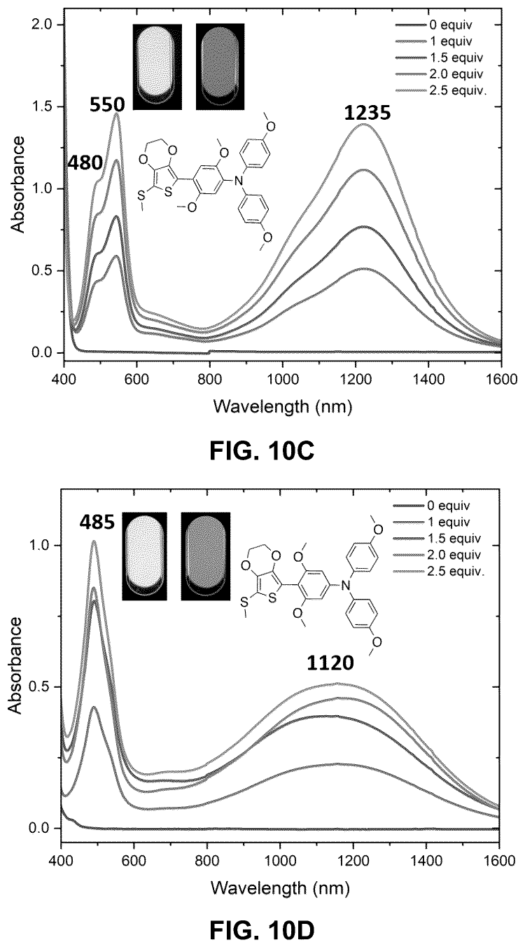

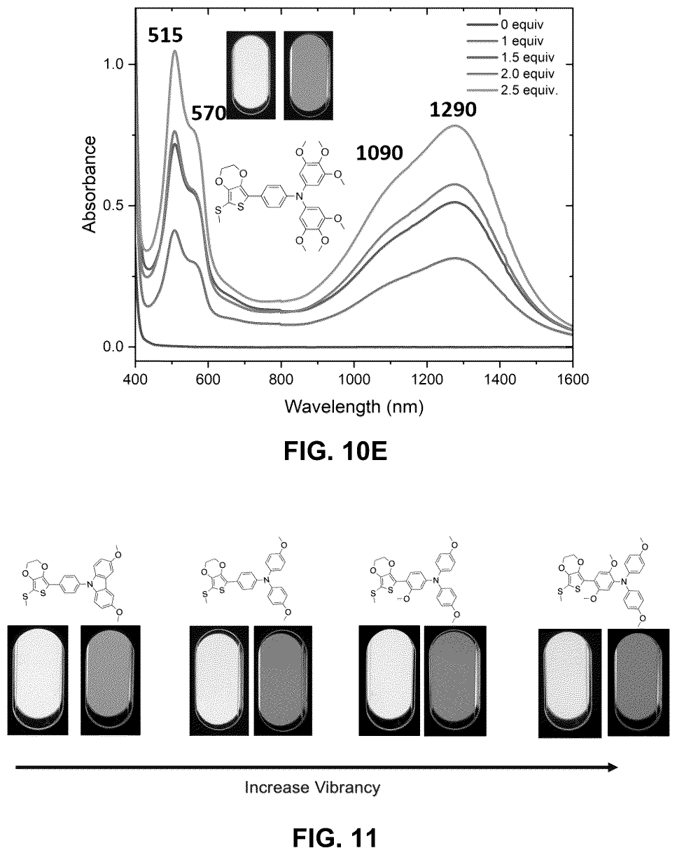

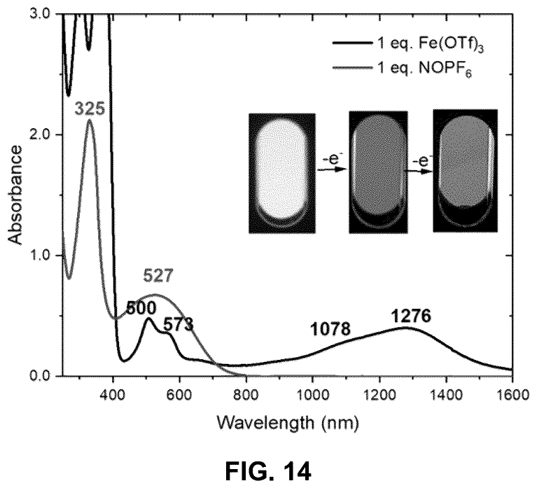

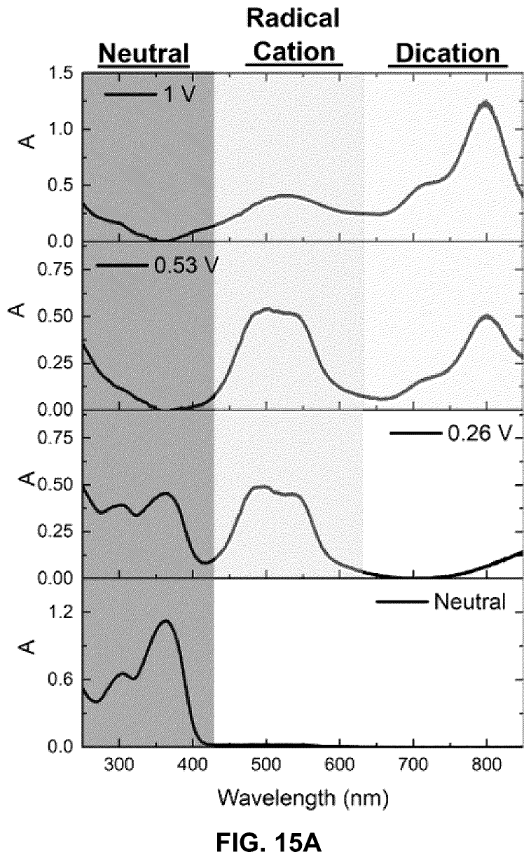

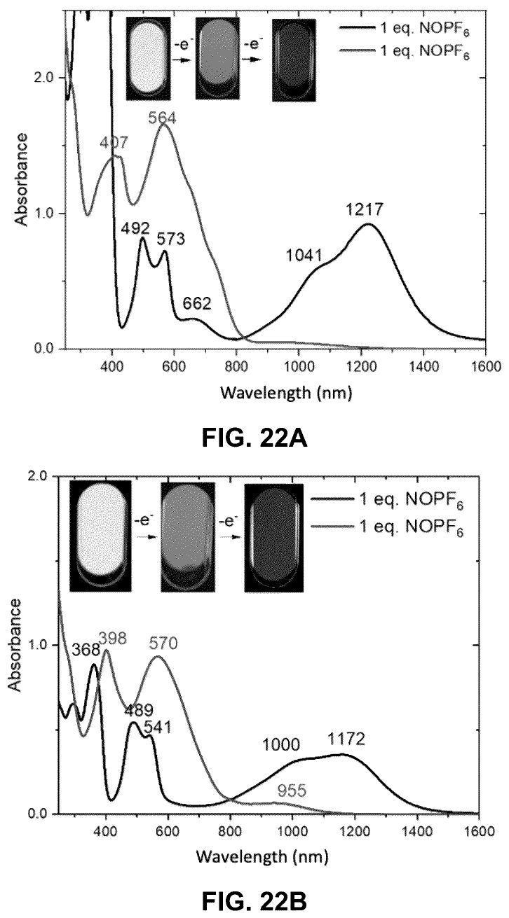

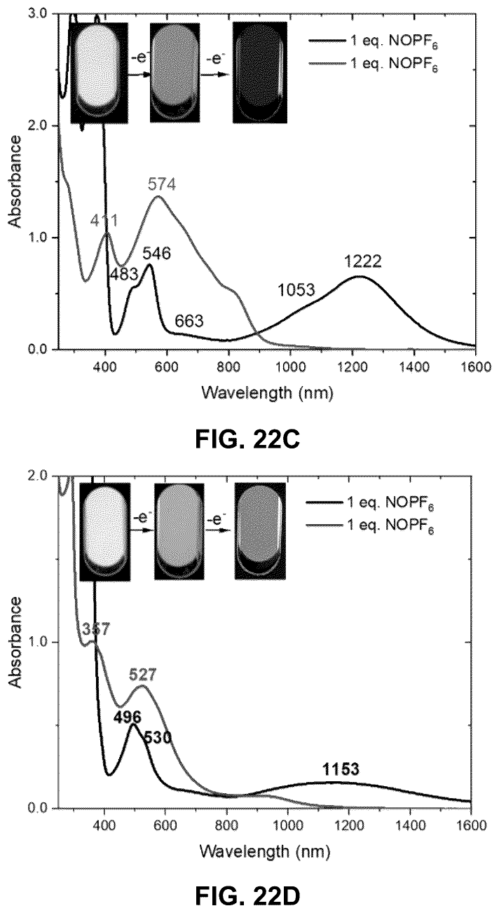

FIGS. 8A-8E show cyclic voltammetry (CV) for the chromophores in Table 1, while FIGS. 9A-9E show differential pulse voltammetry (DPV) for the same molecules. FIGS. 10A-10E show color generation by forming the cation radicals. FIGS. 7A and 7F-7I show a spectroelectrochemistry evaluation of the same. FIGS. 22A-22D and 14 show additional and varying colors obtained with titration of Fe(OTf)₃ and addition of NOPF₆.

### Example 4: Chromophore Design Rationale

A select few chromophores are displayed in FIG. 11, where it is shown that carbazole is different from TPA due to the fusion of the phenyl pendant rings. This displays the color pink. However, upon switching to a TPA unit, the vibrancy and contrast increases upon favorable (S—O) steric interactions that occur between the sulfur of the EDOT and specifically the methoxy substituent on the coupled phenyl ring. Vibrancy is further promoted when the methoxy substituent has favorable steric interactions and locks the phenyl pendants on the amine when conjugated into the pi system of the SOMO.

FIG. 18 shows the structures for five unsymmetrical substituted bi-aryl chromophores, each possessing varying degrees of electron density and different steric and electrostatic interactions. These variations are achieved by strategically placing methoxy substituents in specific locations, starting with the adjacent phenylene coupled to the EDOT and extending towards the outer phenylene pendant rings. Detailed synthetic and characterization information can be found in Example. In brief, the synthesis relies on a two-fold Stille coupling process between a mono-brominated triphenylamine derivative and a 2-methylthio-3,4-dioxy-5-tributyltin-thiophene, conducted in the presence of Pd(PPh₃)₄ This process results in the assembly of discrete molecules with moderate final step yields ranging from 43-63%. The methylthio (MeS) 2′ end cap serves to stabilize the cation radical and dication states. This is accomplished by inhibiting polymerization while simultaneously reducing the oxidation potential of the conjugated system via the resonance-donating effect from the sulfur atom.

Incorporating a triphenylamine unit is of particular interest due to its excellent electron-donating nature leading to ease of oxidation attributed to the conjugation of the nitrogen electron lone pair with the phenyl rings. Additionally, there are prevalent strategies for stabilizing TPA generated cation radicals such as 1) adding substituents at the para-position and 2) planarizing the propeller-shaped TPA with a bridge atom. Stability is enhanced in the latter case because the N-centered cation radical is delocalized over the entire planarized π-system. By using these strategies and using triphenylamine as a building block, a deeper understanding of the electronic structure and the generation of charged species can be gained.

### Structural Analysis

To elucidate the impact of structural modification on the properties of these systems, OFT at the mPW1 PBE/cc-PVDZ level with inclusion of dichloromethane through the conductor polarizable continuum model (CPCM) was performed. Optimized geometries for the neutral, cation radical as well as the dication were generated and analyzed. Table 2 shows the results of these optimizations along with the stabilizing (S▪▪▪O, H▪▪▪O) and destabilizing (O▪▪▪O) non-covalent bonding interactions which were less than or equal to the van der Waals radii (O▪▪▪H˜2.60 Å, O▪▪▪S ∞3.3 ●, O▪▪▪O˜2.8 Å) are shown.

TABLE 2
The neutral (0), cation radical (+1) and dication (+2) geometric results calculated for all
TPA systems with representative non-covalent bonding interactions shown below

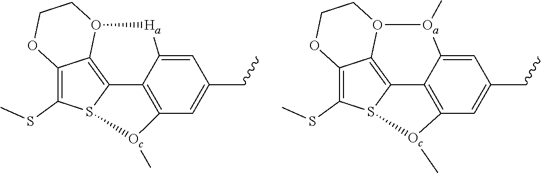

| Charge | | TPA 1 | TPA 2 | TPA 3 | TPA 4 | TPA 5 |
|---|---|---|---|---|---|---|
| 0 | O—Hₐ | 2.28 Å | 2.14 Å | 2.09 Å | N/A | 2.27 Å |
| | S—O_{c} | N/A | 2.68 Å | 2.67 Å | 3.18 Å | N/A |
| | O—Oₐ | N/A | N/A | N/A | 3.02 Å | N/A |
| | Dihedral | 163° | 167° | 170° | 119° | 164° |
| +1 | O—Hₐ | 2.20 Å | 2.09 Å | 2.07 Å | N/A | 2.20 Å |
| | S—O_{c} | N/A | 2.61 Å | 2.62 Å | 2.73 Å | N/A |
| | O—Oₐ | N/A | N/A | N/A | 2.60 Å | N/A |
| | Dihedral | 178° | 180° | 179° | 146° | 178° |
| +2 | O—Hₐ | 2.18 Å | 2.07 Å | 2.05 Å | N/A | 2.18 Å |
| | S—O_{c} | N/A | 2.58 Å | 2.58 Å | 2.59 Å | N/A |
| | O—Oₐ | N/A | N/A | N/A | 2.53 Å | N/A |
| | Dihedral | 178° | 178° | 179° | 157° | 178° |

In all cases, oxidation produces a more planar geometry, which is evidenced by the dihedral angles as well as reducing the distance between the atoms involved in non-covalent bonding. The planarity ranking was TPA 3>TPA 2>TPA 1˜TPA 5>TPA 4. The unsubstituted central phenyl ring for both TPA 1 and TPA 5 produced nearly the same central geometries. On the other hand, the torsional strain provided by the two methoxy groups next to the EDOT ring coupled with the destabilizing O▪▪▪O interaction resulted in a large out-of-plane twist for TPA 4. Both TPA 2 and TPA 3 produced nearly identical geometries with only slightly more planar neutral geometry for TPA 3 likely due to the electron density provided by the additional methoxy group.

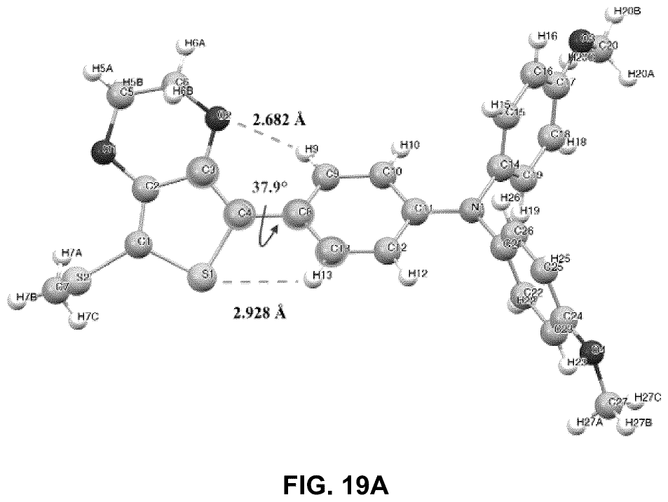

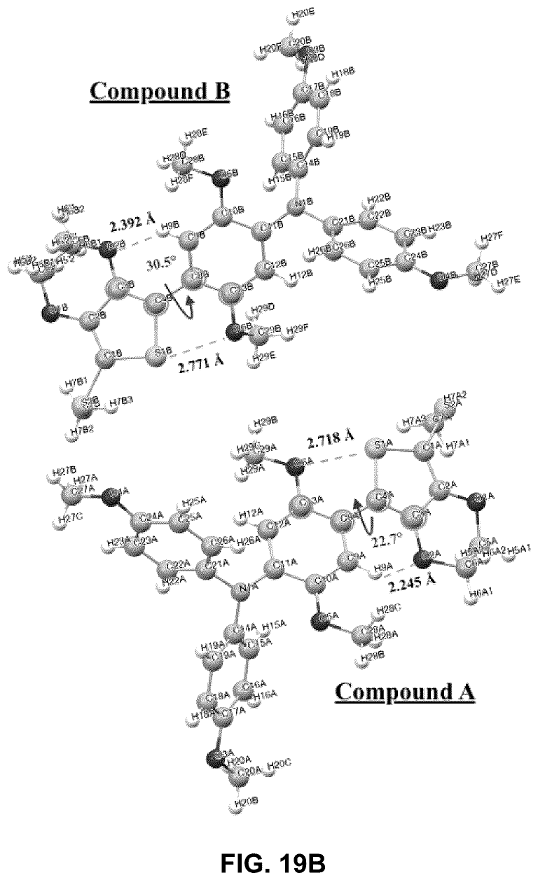

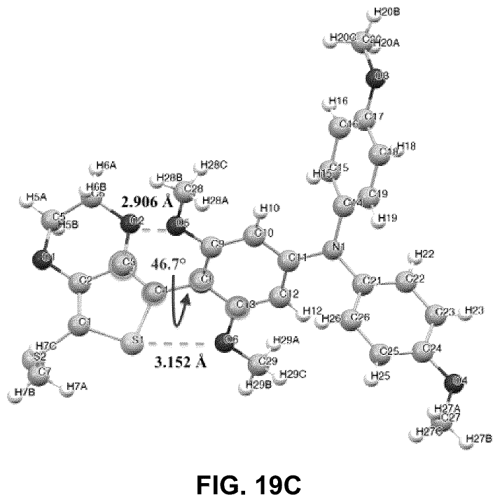

Single crystals suitable for X-ray structure determination were grown by slow liquid-liquid diffusion from a binary solvent by dissolving the compounds in dichloromethane and layering a poor solvent of hexanes. The crystals formed fine-needle rods or blocks that maintained a pale yellow color. As shown in FIGS. 19A-19C, the structures obtained are mostly ordered. FIG. 19A displays TPA 1 where the structure contains a steric interaction between the phenylene hydrogen and sulfur atom on the EDOT aryl rings resulting in a torsional angle of 37.8° (which corresponds to an angle of 142.2° in the computations) and an O—H interatomic distance to be 2.923 Å. FIG. 19B displays TPA 3 which contains two crystallographically independent molecules in the asymmetric unit resulting in the —O1X—C5X—C6X—O2X— moiety to be disordered over two orientations. For the compound A variant in FIG. 19B, the torsional twist is 22.7° and two interactions occur, one being at S1A-O6A resulting in an atomic distance measurement of 2.718 Å, and another on the EDOT ethylene bridge (O—H) measured at 2.245 Å. The compound B variant has a torsional twist of 30.5° with two interactions occurring, one being at S1B—O6B resulting in an atomic distance measurement of 2.771 Å, and another on the EDOT ethylene bridge consisting of an O—H interaction measured at 2.392 Å. This decreases the distance due to stabilizing noncovalent steric interactions. In FIG. 19C, the compound TPA 4 displays an asymmetric unit containing one partially occupied lattice DCM solvent molecule that is found at one site of threefold axial symmetry (and thus must be disordered). The compound has the largest torsional strain due to the repulsive interaction between the adjacent oxygen atoms on the phenylene ring and the ethylene bridge of EDOT. This results in a greater torsional strain of 46.7° and a O—S interatomic distance of 3.152 Å while the steric interaction between O—O is shorter at 2.906 Å (this influences the behavior of this molecule discussed later). In comparison to using DFT as a guide, there is an overall calculation variation of 15-20° in the torsional angle and 0.1-0.4 Å in the atomic distance, which may be due to packing influences in the solid state.

### Electrochemical Evaluation

The position of a methoxy substituent on the chromophore not only impacts its structure but also subsequently influences its redox properties. This stems from both the impact on electron density and steric interactions that can significantly affect the energy required for the formation of oxidized species. To understand the redox process, solution electrochemistry was performed in 0.5 M tetrabutylammonium hexafluorophosphate (NBu₄PF₆)/dichloromethane (DCM) solution. Cyclic voltammetry (CV) and differential pulse voltammetry (DPV) were used to probe the onset of oxidation by extracting the faradaic current and extracting the half-wave potentials (E_{1/2}).

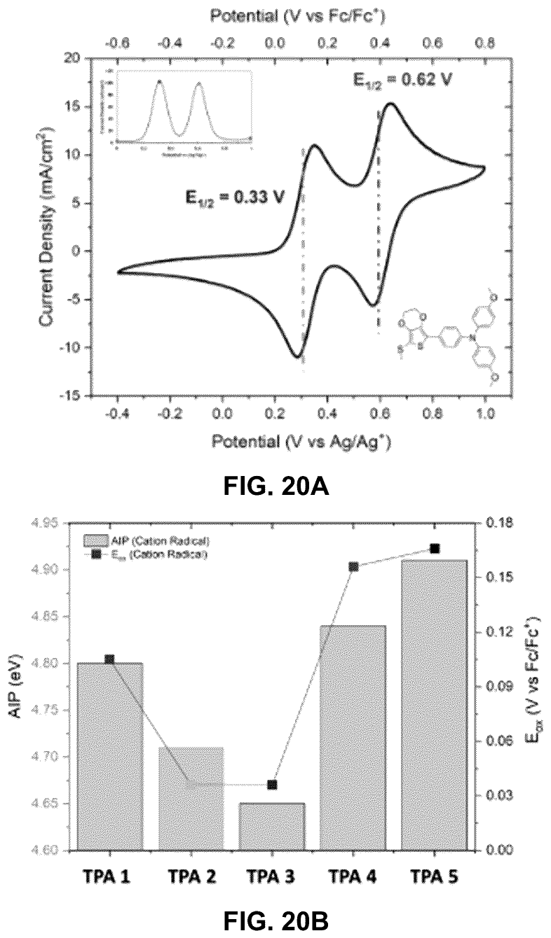

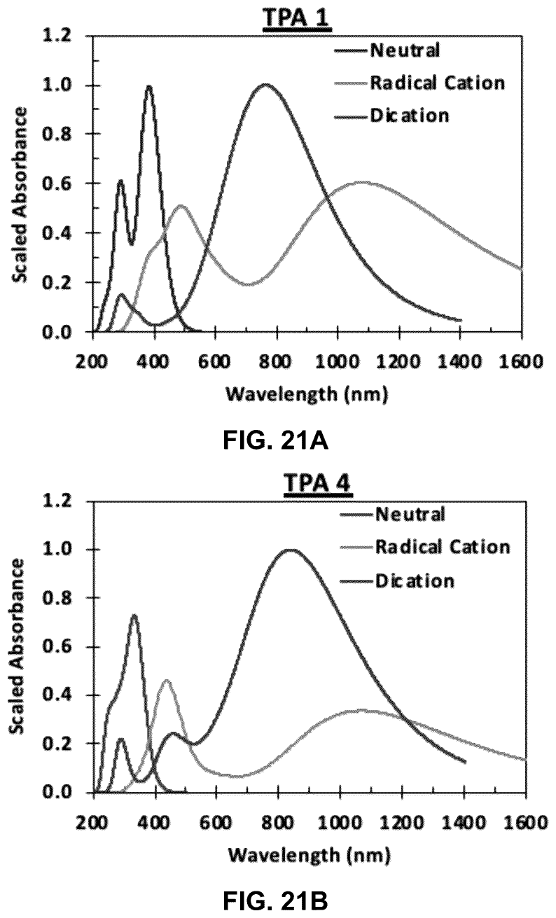

Well-resolved quasi-reversible events were observed in the formation of the cation radical and dication for all five chromophores via CV seen in FIGS. 8A-8E. FIG. 20A displays the CV and the inset contains the DPV (FIGS. 9A-9E) of TPA 1 with the extracted E_{1/2} of the cation radical and dication species generated in Table 3, which summarizes the electrochemical results for all five chromophores showing the potential difference between the two redox processes.

TABLE 3
electrochemical properties of all five chromophores with ΔE representing
potential difference between first and second oxidation peaks

| | E_{1/2} | E_{1/2} | |
|---|---|---|---|
| | (V, first | (V, second | |
| Chromophore | oxidation peak) | oxidation peak) | ΔE (V) |
| TPA 1 | 0.33 | 0.62 | 0.29 |
| TPA 2 | 0.26 | 0.53 | 0.27 |
| TPA 3 | 0.26 | 0.49 | 0.23 |
| TPA 4 | 0.38 | 0.52 | 0.14 |
| TPA 5 | 0.39 | 0.63 | 0.24 |

TPA 1 represents the standard in the set where substitution at the para position of the pendant phenylene rings remains constant with a methoxy substituent. This serves to add electron density and cap the reactive end inhibiting radical coupling. TPA 1 oxidizes to the cation radical at an E1/2 of 0.33 V Ag/Ag⁺, while the dication forms at 0.62 V Ag/Ag⁺ with the highest potential separation of the two redox processes at 0.29 V. TPA 2 and TPA 3 both possessed the lowest E_{1/2} to form the cation radical at 0.26 V where the non-covalent bonding interaction (O•••S) results in conformational locking. It is interesting to note that adding an additional O•••H intramolecular interaction between the methoxy in the 5′ position on the phenylene and aryl pendant on the triphenylamine lowers the E_{1/2} of the dicationic species to 0.49 V. When the design series is shifted to incorporating repulsive (O•••O) steric interactions found in TPA 4, the E_{1/2} of the cation radical rises to 0.38 V and the dication lowers to 0.52 V resulting in the smallest charge state separation in the defined set at 0.14 V. Increasing the electron richness of the substituent at the meta position of the triphenylamine (TPA 5) resulted in increasing E1/2 to form the cation radical species to the highest at 0.39 V, but had no substantial impact on the dication. This demonstrates the importance of how controlled structures tailors redox potentials. Strategic substituent placement, such as attractive interactions that involve conformational locking, can result in lowering the oxidation potential of both charged species. Interactions that involve repulsive steric interactions, and lead to a torqued system, raises the oxidation potential to form the cation radical, with less effect on dication formation leading to a smaller potential difference between the two redox processes. In order to find the highest potential separation of charged states, a balance between electron density and steric interactions must be considered.

FIG. 20B compares the calculated Adiabatic Ionization Potential (AIP) with the electrochemical measurement of the ionization potential attained from DPV measurements (FIGS. 9A-9E), demonstrating there is a strong correlation between theory and experiment for the first oxidation (cation radical state). However, this same trend was not observed for AIP modeling of the dication state. It has been reported that dications require sophisticated treatment of electron correlation in order to accurately compute interaction energies for dication complexes without overestimation.

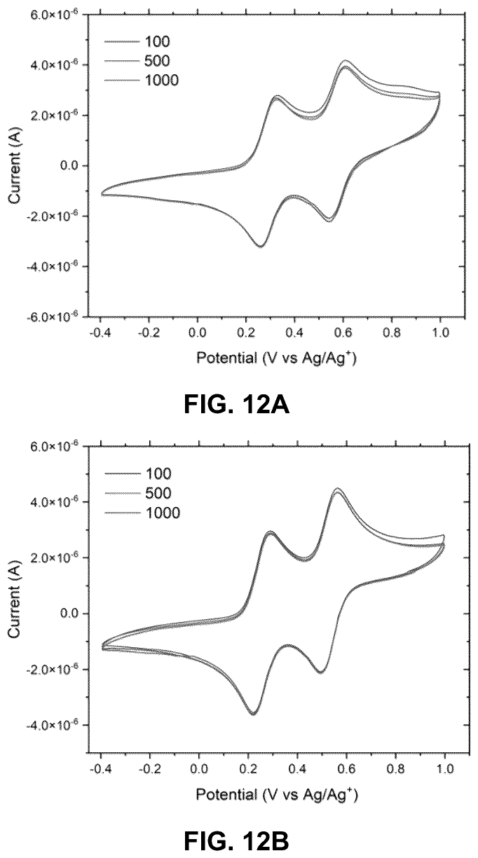

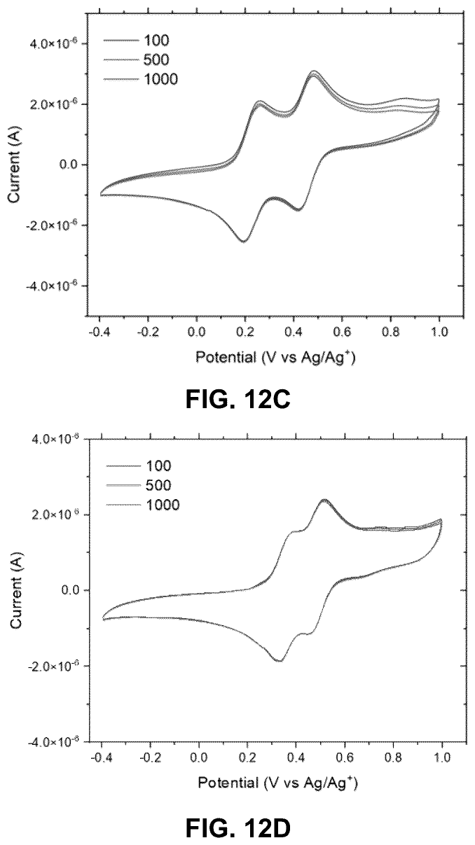

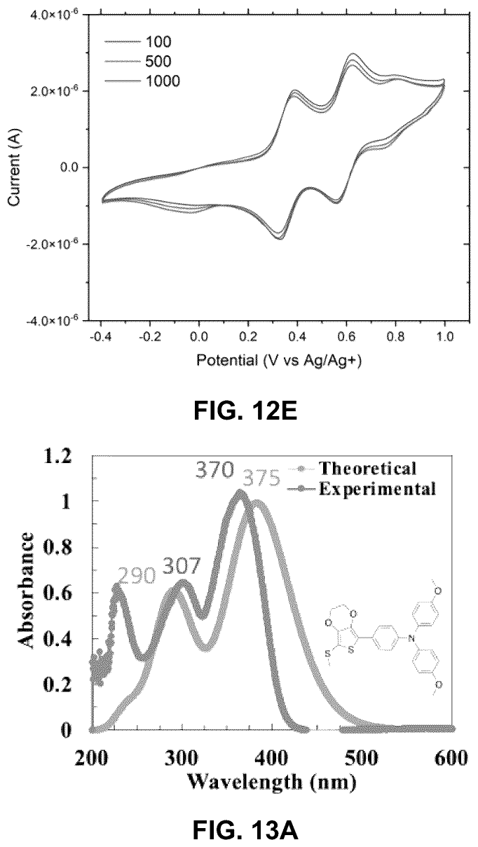

Cyclic stability tests on all five chromophores, as shown in FIGS. 12A-12E, were performed up to 1000 cycles by at a scan rate of 150 mV/s between the potentials of −0.4 to 1.0 V vs Ag/Ag⁺. Satisfactory stability is demonstrated by the near identical CV profiles of all chromophores with no more than 5-15% current loss occurring at higher applied oxidation potentials (>0.8 V).

### Chemical Generation of the Cation Radical and Subsequently the Dication

To elucidate the origin of the spectral peaks for each of the species, a time-dependent DFT treatment was performed in which the lowest lying 15 excited states were calculated. As an illustration, FIGS. 7A-7B show UV-Vis spectra for A) TPA 1 and B) TPA 4. The excited states for each oxidized species are shown in Table 4. Both set of spectra show a neutral state with dominant absorption in the UV, along with two cation radical peaks and a dominant dication peak through the visible and near IR. These same trends are also demonstrated by the other three systems (Tables 5-9). A close examination of each charged state in the following discussion provides insight into how the location and number of methoxy substituent impact the spectra which leads to changes in color.

TABLE 4
Excited analysis results for all three charges of exemplary systems under examination

| | Neutral | | | Radical Cation | | | | Dication | | | |
|---|---|---|---|---|---|---|---|---|---|---|---|
| | Excited States | | | Excited States (nm) | | | | Excited States | | | |
| Substituent | λ₁ (f) | λ₂ (f) | λ₃ (f) | λ₁ (f) | λ₂ (f) | λ₃ (f) | λ₄ (f) | λ₁ (f) | λ₂ (f) | λ₃ (f) | λ₄ (f) |
| —H | 384 | 291 | N/A | 1081 | 622 | 483 | 393 | 792 | 709 | 612 | N/A |
| | (1.0105) | (0.1387) | | (0.6050) | (0.1646) | (0.4105) | (0.2359) | (1.3275) | (0.1740) | (0.3704) | |
| | H → | H → | | Sb → | Sb-2 → | Sb-4 → | Sb → | H → | H-2 → | H-1 → | |
| | L | L + 4 | | Lb | Lb | Lb/Sa → | Lb + 1 | L | L | L | |
| | | | | | | Sb | | | | | |
| -2-OCH₃ | 377 | 304 | N/A | 1044 | N/A | 463 | N/A | 806 | 620 | 541 | N/A |
| | (1.1216) | (0.2359) | | (0.5620) | | (0.6730) | | (1.2828) | (0.2848) | (0.1041) | |
| | H → | H → | | Sb → | | Sa → | | H → | H-1 → | H-3 → | |
| | L | L + 2 | | Lb | | La | | L | L | L | |
| -2,5-OCH₃ | 402 | 299 | N/A | 1087 | 707 | 505 | 401 | 814 | 729 | 645 | 618 |
| | (0.8582) | (0.3426) | | (0.5101) | (0.1234) | (0.2644) | (0.1418) | (1.0099) | (0.1957) | (0.1989) | (0.2896) |
| | H → | H → | | Sb → | Sb-2 → | Sb-4 → | Sb → | H → | H-1 → | H-3 → | H-2 → |
| | L | L + 2 | | Lb | Lb | Lb/Sa → | Lb + 1 | L | L | L | L |
| | | | | | | Sb | | | | | |
| -2,6-OCH₃ | 339 | 297 | N/A | 1125 | 447 | 445 | N/A | 852 | 631 | 444 | N/A |
| | (0.9596) | (0.2748) | | (0.5121) | (0.1105) | (0.4699) | | (1.1125) | (0.2206) | (0.2199) | |
| | H → | H → | | Sb → | Sb-5 → | Sa → | | H → | H-1 → | H-5 → | |
| | L | L + 2 | | Lb | Lb | La | | L | L | L | |
| -3,4,5-OCH₃ | 381 | 308 | 290 | 1072 | N/A | 473 | N/A | 864 | 825 | 676 | 646 |
| | (0.9455) | (0.3308) | (0.2264) | (0.6041) | | (0.6091) | | (0.8679) | (0.4926) | (0.2492) | (0.1735) |
| | H → | H → | H-2 → | Sb → | | Sa → | | H → | H-1 → | H-2 → | H-3 → |
| | L | L + 2 | L | Lb | | La | | L | L | L | L |

TABLE 5
Lowest Excited States for TPA 1

| | Neutral | | | Radical Cation | | | Dication | | |
|---|---|---|---|---|---|---|---|---|---|
| Excited | Energy | λ | | Energy | λ | | Energy | λ | |
| state | (eV) | (nm) | f | (eV) | (nm) | f | (eV) | (nm) | f |
| 1 | 3.23 | 384 | 1.0105 | 1.15 | 1081 | 0.6050 | 1.57 | 792 | 1.3275 |
| 2 | 3.63 | 342 | 0.0306 | 1.74 | 714 | 0.0047 | 1.75 | 709 | 0.1740 |
| 3 | 4.09 | 303 | 0.2018 | 1.99 | 622 | 0.1646 | 2.03 | 612 | 0.3704 |
| 4 | 4.16 | 298 | 0.0335 | 2.34 | 528 | 0.0613 | 2.52 | 492 | 0.0458 |
| 5 | 4.26 | 291 | 0.1387 | 2.35 | 483 | 0.4105 | 2.66 | 467 | 0.0004 |
| 6 | 4.32 | 287 | 0.1799 | 2.58 | 480 | 0.0009 | 2.81 | 441 | 0.0267 |
| 7 | 4.43 | 280 | 0.0838 | 2.75 | 451 | 0.0059 | 2.96 | 418 | 0.0022 |
| 8 | 4.55 | 273 | 0.0552 | 2.88 | 430 | 0.0010 | 3.57 | 347 | 0.0512 |
| 9 | 4.83 | 257 | 0.0221 | 3.15 | 393 | 0.2359 | 3.71 | 334 | 0.0665 |
| 10 | 4.89 | 253 | 0.0281 | 3.39 | 366 | 0.0152 | 3.92 | 316 | 0.0025 |
| 11 | 5.01 | 247 | 0.0026 | 3.44 | 361 | 0.0448 | 4.24 | 292 | 0.1237 |
| 12 | 5.05 | 246 | 0.0024 | 3.50 | 355 | 0.0057 | 4.28 | 290 | 0.0209 |
| 13 | 5.18 | 239 | 0.0184 | 3.53 | 351 | 0.0105 | 4.29 | 289 | 0.0154 |
| 14 | 5.21 | 238 | 0.0119 | 3.75 | 331 | 0.0225 | 4.33 | 286 | 0.0594 |
| 15 | 5.23 | 237 | 0.0549 | 3.87 | 321 | 0.0034 | 4.35 | 285 | 0.0110 |

TABLE 6
Lowest Excited States for TPA 2

| | Neutral | | | Radical Cation | | | Dication | | |
|---|---|---|---|---|---|---|---|---|---|
| Excited | Energy | λ | | Energy | λ | | Energy | λ | |
| state | (eV) | (nm) | f | (eV) | (nm) | f | (eV) | (nm) | f |
| 1 | 3.29 | 377 | 1.1216 | 1.19 | 1044 | 0.5620 | 1.58 | 806 | 1.2828 |
| 2 | 3.69 | 336 | 0.0311 | 1.64 | 756 | 0.0043 | 1.75 | 708 | 0.0250 |
| 3 | 4.08 | 304 | 0.2359 | 2.07 | 598 | 0.0234 | 2.00 | 620 | 0.2848 |
| 4 | 4.13 | 300 | 0.0188 | 2.15 | 576 | 0.0859 | 2.29 | 541 | 0.1041 |
| 5 | 4.25 | 292 | 0.0627 | 2.40 | 517 | 0.0372 | 2.54 | 489 | 0.0821 |
| 6 | 4.42 | 280 | 0.1024 | 2.69 | 461 | 0.6730 | 2.77 | 447 | 0.0109 |
| 7 | 4.44 | 279 | 0.0586 | 2.87 | 432 | 0.0261 | 2.86 | 433 | 0.0964 |
| 8 | 4.54 | 273 | 0.0589 | 2.96 | 418 | 0.0023 | 3.57 | 347 | 0.0358 |
| 9 | 4.81 | 258 | 0.0322 | 3.18 | 389 | 0.0804 | 3.73 | 332 | 0.0227 |
| 10 | 4.91 | 252 | 0.0385 | 3.41 | 364 | 0.0203 | 3.97 | 313 | 0.0063 |
| 11 | 5.03 | 247 | 0.1002 | 3.41 | 364 | 0.0024 | 4.27 | 290 | 0.0178 |
| 12 | 5.07 | 245 | 0.0004 | 3.57 | 347 | 0.0058 | 4.29 | 289 | 0.0423 |
| 13 | 5.14 | 241 | 0.0238 | 3.64 | 340 | 0.0051 | 4.36 | 284 | 0.0816 |
| 14 | 5.22 | 238 | 0.1257 | 3.75 | 330 | 0.0155 | 4.38 | 283 | 0.0481 |
| 15 | 5.27 | 235 | 0.0075 | 3.88 | 320 | 0.0160 | 4.41 | 281 | 0.0436 |

TABLE 7
Lowest Excited States for TPA 3

| | Neutral | | | Radical Cation | | | Dication | | |
|---|---|---|---|---|---|---|---|---|---|
| Excited | Energy | λ | | Energy | λ | | Energy | λ | |
| state | (eV) | (nm) | f | (eV) | (nm) | f | (eV) | (nm) | f |
| 1 | 3.08 | 402 | 0.8582 | 1.14 | 1087 | 0.5101 | 1.52 | 814 | 1.0099 |
| 2 | 3.69 | 336 | 0.0245 | 1.53 | 809 | 0.0252 | 1.70 | 729 | 0.1957 |
| 3 | 3.91 | 317 | 0.1180 | 1.83 | 679 | 0.0006 | 1.92 | 645 | 0.1898 |
| 4 | 4.14 | 299 | 0.3426 | 2.04 | 607 | 0.1234 | 2.01 | 618 | 0.2896 |
| 5 | 4.18 | 296 | 0.0376 | 2.45 | 505 | 0.2633 | 2.57 | 482 | 0.0339 |
| 6 | 4.34 | 286 | 0.2019 | 2.61 | 474 | 0.3286 | 2.72 | 456 | 0.0175 |
| 7 | 4.34 | 286 | 0.0600 | 2.79 | 444 | 0.0099 | 2.92 | 424 | 0.0336 |
| 8 | 4.46 | 278 | 0.0654 | 2.94 | 421 | 0.0046 | 3.52 | 353 | 0.0294 |
| 9 | 4.69 | 264 | 0.0151 | 3.10 | 401 | 0.1418 | 3.66 | 339 | 0.0361 |
| 10 | 4.73 | 262 | 0.0729 | 3.35 | 370 | 0.0058 | 3.97 | 312 | 0.0065 |
| 11 | 4.81 | 258 | 0.0647 | 3.40 | 365 | 0.0316 | 4.08 | 304 | 0.0913 |
| 12 | 4.98 | 249 | 0.0013 | 3.47 | 357 | 0.0045 | 4.17 | 298 | 0.0931 |
| 13 | 5.10 | 243 | 0.0361 | 3.61 | 343 | 0.0064 | 4.24 | 292 | 0.0924 |
| 14 | 5.10 | 243 | 0.0889 | 3.54 | 340 | 0.0150 | 4.33 | 286 | 0.0171 |
| 15 | 5.24 | 236 | 0.0164 | 3.77 | 329 | 0.0020 | 4.37 | 283 | 0.0301 |

TABLE 8
Lowest Excited States for TPA 4

| | Neutral | | | Radical Cation | | | Dication | | |
|---|---|---|---|---|---|---|---|---|---|
| Excited | Energy | λ | | Energy | λ | | Energy | λ | |
| state | (eV) | (nm) | f | (eV) | (nm) | f | (eV) | (nm) | f |
| 1 | 3.66 | 339 | 0.8118 | 1.10 | 1125 | 0.5121 | 1.45 | 852 | 1.1125 |
| 2 | 3.84 | 324 | 0.0285 | 1.49 | 830 | 0.0017 | 1.63 | 762 | 0.0080 |
| 3 | 4.17 | 297 | 0.2748 | 1.67 | 741 | 0.0021 | 1.87 | 662 | 0.0215 |
| 4 | 4.20 | 295 | 0.0330 | 2.14 | 580 | 0.0749 | 1.97 | 631 | 0.2206 |
| 5 | 4.34 | 286 | 0.0652 | 2.15 | 576 | 0.0167 | 2.51 | 495 | 0.0797 |
| 6 | 4.54 | 273 | 0.0040 | 2.77 | 447 | 0.1105 | 2.72 | 455 | 0.0031 |
| 7 | 4.56 | 272 | 0.1334 | 2.79 | 445 | 0.0009 | 2.79 | 444 | 0.2199 |
| 8 | 4.68 | 265 | 0.1168 | 2.94 | 421 | 0.0004 | 3.31 | 275 | 0.0040 |
| 9 | 4.96 | 250 | 0.0835 | 3.01 | 412 | 0.0128 | 3.54 | 350 | 0.0290 |
| 10 | 4.99 | 248 | 0.0502 | 3.27 | 377.8 | 0.0002 | 3.90 | 318 | 0.0114 |
| 11 | 5.12 | 242 | 0.1567 | 3.39 | 365 | 0.0094 | 4.09 | 303 | 0.0009 |
| 12 | 5.15 | 241 | 0.0100 | 3.51 | 353 | 0.0396 | 4.25 | 292 | 0.0607 |
| 13 | 5.22 | 238 | 0.0101 | 3.56 | 348 | 0.0088 | 4.29 | 289 | 0.1729 |
| 14 | 5.24 | 237 | 0.0057 | 3.72 | 333 | 0.0203 | 4.36 | 284 | 0.0174 |
| 15 | 5.28 | 235 | 0.0022 | 3.73 | 333 | 0.0043 | 4.39 | 203 | 0.0038 |

TABLE 9
Lowest Excited States for TPA 5

| | Neutral | | | Radical Cation | | | Dication | | |
|---|---|---|---|---|---|---|---|---|---|
| Excited | Energy | λ | | Energy | λ | | Energy | λ | |
| state | (eV) | (nm) | f | (eV) | (nm) | f | (eV) | (nm) | f |
| 1 | 3.25 | 381 | 0.9455 | 1.16 | 1072 | 0.6041 | 1.43 | 864 | 0.8679 |
| 2 | 3.88 | 320 | 0.0446 | 1.59 | 779 | 0.0048 | 1.50 | 825 | 0.4926 |
| 3 | 4.02 | 308 | 0.3308 | 1.65 | 752 | 0.0019 | 1.69 | 734 | 0.0129 |
| 4 | 4.28 | 290 | 0.2264 | 1.90 | 653 | 0.0761 | 1.83 | 676 | 0.2494 |
| 5 | 4.37 | 284 | 0.0985 | 2.08 | 597 | 0.0059 | 1.92 | 646 | 0.1735 |
| 6 | 4.42 | 280 | 0.0235 | 2.33 | 531 | 0.0369 | 2.50 | 497 | 0.0003 |
| 7 | 4.52 | 274 | 0.0077 | 2.59 | 479 | 0.0006 | 2.63 | 472 | 0.0576 |
| 8 | 4.58 | 271 | 0.0210 | 2.63 | 472 | 0.6091 | 2.86 | 434 | 0.0016 |
| 9 | 4.68 | 265 | 0.0272 | 2.90 | 427 | 0.0095 | 2.86 | 433 | 0.0016 |
| 10 | 4.77 | 260 | 0.0203 | 3.12 | 397 | 0.0947 | 3.42 | 363 | 0.0585 |
| 11 | 4.96 | 250 | 0.0020 | 3.17 | 390 | 0.0052 | 3.57 | 348 | 0.0585 |
| 12 | 4.96 | 250 | 0.0058 | 3.27 | 379 | 0.0236 | 3.60 | 345 | 0.0577 |
| 13 | 5.01 | 248 | 0.0135 | 3.34 | 372 | 0.0227 | 3.86 | 321 | 0.0035 |
| 14 | 5.13 | 242 | 0.0226 | 3.48 | 356 | 0.0028 | 3.97 | 313 | 0.0534 |
| 15 | 5.16 | 240 | 0.0662 | 3.56 | 348 | 0.0064 | 3.99 | 311 | 0.0016 |

In the case of the neutral species, there were two significant transitions present. The most prominent peak in all cases resulted from a transition from the highest occupied molecular orbital (HOMO, H) to the lowest unoccupied molecular orbital (LUMO, L). The wavelengths corresponding to this state ranged from 339 nm (-2,6-OCH₃—, referred to as TPA 4) which possessed the most twisted out-of-plane geometry to 402 nm (-2,5-OCH₃—, TPA 3) the most planar system. The second excited state, which was primarily the result of the H→L+2 transition only differed by 9 nm. The exception was produced by the unsubstituted (—H—, TPA 1) system which was 6 nm blue-shifted relative to the other systems and was due to the H→L+4 transition. In all cases, except for the TPA 3 system, the calculated neutral state was outside the visible range and as desired for the compounds to appear transparent.

Cation radical excited states involve the excitation of an alpha (α) electron or a beta (β) electron and as such each electron type will be identified for a given excitation. In all cases, the first cation radical excited state occurred from a singly occupied molecular orbital (SOMO) β electron to the L_{β} level and ranged from 1044 nm (-2-OCH₃—, (TPA 2)) to 1125 nm (-2,6-OCH₃—, TPA 4). Both —H— (TPA 1) and -2,5-OCH₃— (TPA 3) possessed an excited state that resulted from the S_{β}→L_{β} transition and differed by 85 nm that would ultimately affect the color these molecules would exhibit as cation radicals. Additionally, they exhibited a third transition produced primarily from S_{β}-4→L_{β} with a large contribution from S_{α}→L_{α} in which Δλ was 22 nm. For the remaining three, the higher energy peak was produced by the S_{α}→L_{α} which ranged from 445 nm to 473 nm and was at most 60 nm lower than the other set. Finally, there was another excited present for the TPA 1, TPA 3, and TPA 4 species which possessed lower oscillator strengths, but nonetheless resulted in a widening of the higher energy peak. Overall, the differences between these peaks, coupled with those contributed from lower energy transitions, suggested that there would be a difference in coloration within this set of structures.

Finally, the presence of a dication for all systems was observed computationally. For the lower energy peak, an electron was excited from the HOMO to the LUMO level in which the wavelength ranged from 792 nm (—H—, TPA 1) to 864 nm (-3,4,5-OCH₃—, TPA 5). The trimethoxy species exhibited a close lying lower energy excitation at 825 (f=0.4926) which resulted from a H-1→L. The other four species also exhibited this transition which ranged from 612 nm (—H—) to 729 nm (2,5-OCH₃—). There were additional excited states which contributed to the overall width of the spectral peaks. Given the similarity in the curvilinear Gaussian fits to the spectral data, the color contrast in the dication state is expected to be small. To verify the validity of these predictions experimental studies were performed.

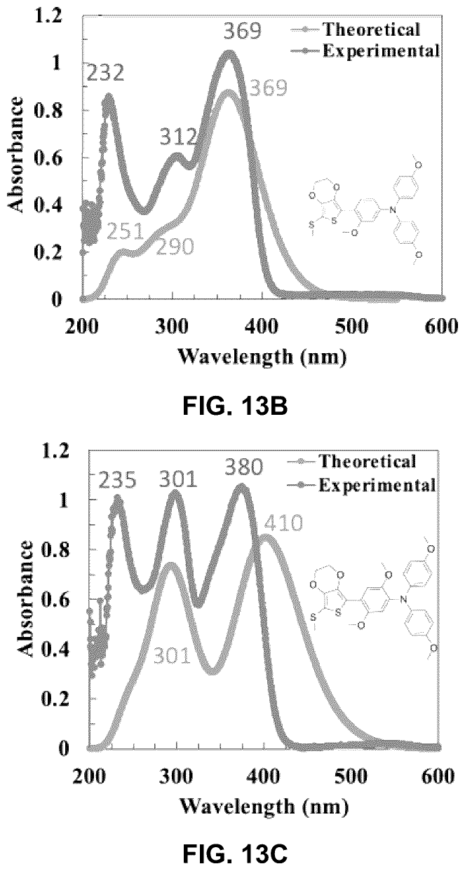

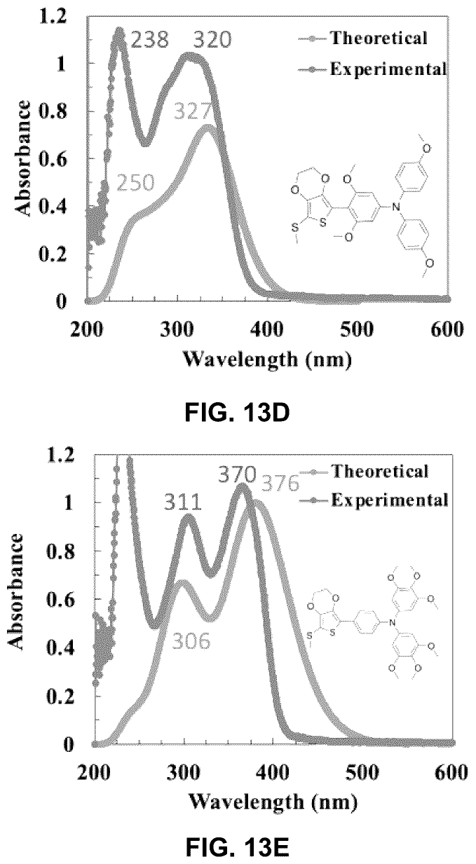

In previous studies, to assess the transmissivity of the neutral state and color vibrancy of the charged state, ACE chromophores were chemically oxidized using iron (III) trifluoromethanesulfonate (Fe(OTf)₃) to generate the cation radical, followed by subsequent chemical oxidation with NOPF6 to reach the dication state. Each chromophore was dissolved at a concentration of 250 μM, titrated with a respected 1 equivalent of oxidant, and left to react overnight with Fe(OTf)₃, but only 1 hour with NOPF6. The neutral spectra of all chromophores show excellent correlation between the computationally determined λₘₐₓ and those determined experimentally, FIGS. 13A-13E. The experimental results, which include sequential solution oxidation and the photographed color transitions are shown in FIGS. 22A-22D.

All chromophores exhibit a neutral absorption λₘₐₓ in the UV (attributed to the π-π* transition), resulting in a highly transmissive and colorless appearance (FIGS. 13A-13E). Upon the addition of an oxidant, the neutral absorbance depletes while new transitions are generated, resulting in highly vibrant and bright colors. FIGS. 22A-22D and FIG. 14 display the absorption profiles after the initial titration of 1 equivalent of Fe(OTf)₃ (black curve), along with an additional 1 equivalent of NOPF6 (red curve). When comparing the cation radical spectra, TPA 1 (λₘₐₓ=510 and 580 nm), TPA 2 (λₘₐₓ=500 and 550 nm), TPA 3 (λₘₐₓ=480 and 550 nm), and TPA 5 (λₘₐₓ=500 and 575 nm) all exhibit double absorbance peaks with slight variations in peak height, resulting in a range of vibrant and bright magenta/pink, as discussed later. In contrast, a single HE absorbance transition can be seen for TPA 4 (λₘₐₓ=500 nm) resulting in the generation of the color orange in the set. Regarding the LE transition in the cation radical charged state, all molecules display at least one LE absorbance peaks in the NIR region, as well as harboring a shoulder, with variations in peak intensity compared to the HE. Shifting the LE peak to the NIR is essential, as it allows for a dominant HE transition in the visible region of the spectrum to predominantly influence the perceived color.

Subsequent oxidation with the stronger oxidant NOPF₆ generates a new palette of colors shown in FIGS. 22A-22D and FIG. 14. With the exception of TPA 1, all of the chromophores undergo a drastic reduction in the intensity of the long-wavelength NIR absorption, and two dominant absorption peaks emerge in the visible region, which is attributed to the formation of dication states. Comparing these spectra for TPA 2 (λₘₐₓ=425 and 600 nm), TPA 3 (λₘₐₓ=410 and 600 nm), TPA 4 (λₘₐₓ=400 and 550 nm), and TPA 5 (λₘₐₓ=335 and 500 nm) these two dominant and relatively broad absorbances are found in the range of 400-800 nm. In contrast, the TPA 1 (FIG. 22A) chromophore does not alter its general spectral profile; the intensity of the original absorption peaks increase, but remains in the same position (λₘₐₓ=525, 700, and 1240 nm). This is unusual, especially when compared to TPA 5 (FIG. 14), which generates a similar burgundy color but exhibits a different absorption profile. An alternative situation is observed with TPA 4 (FIG. 22D), where the orange color becomes more saturated as the absorption peak in the NIR decreases. While the cation radicals of each system (except for the TPA 4 derivative), all attain similar colors, this is not the case for the dications where there is a much greater color diversity as the spectra are significantly different.

### Electrochromism

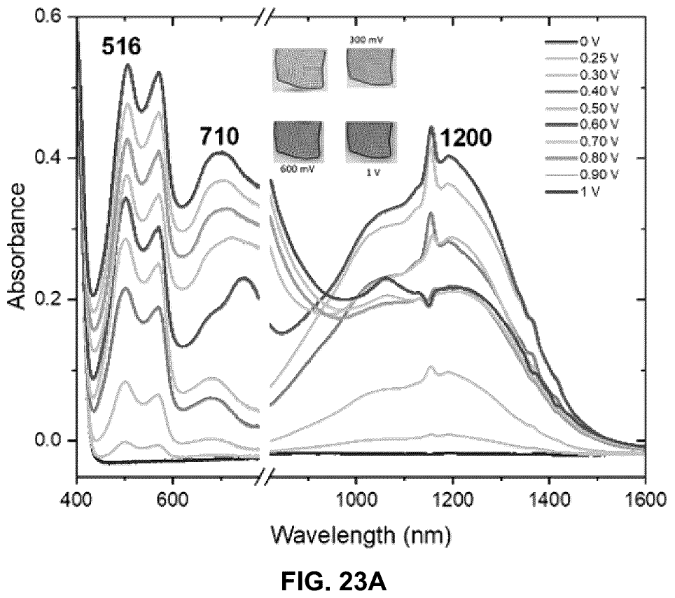

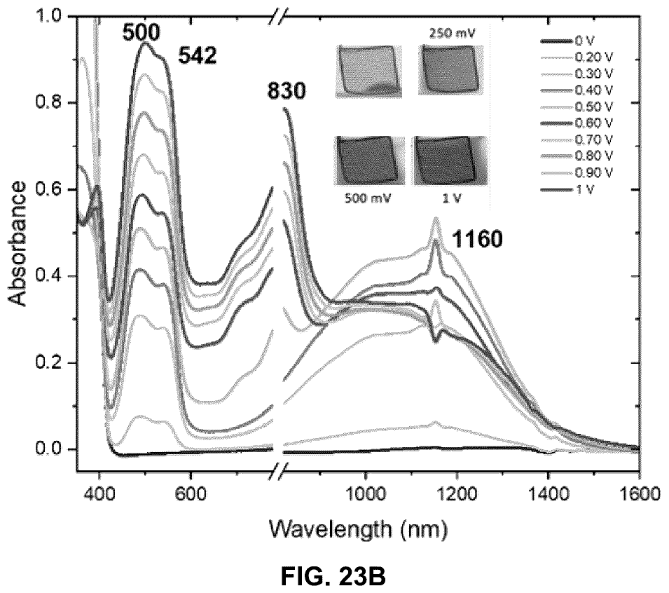

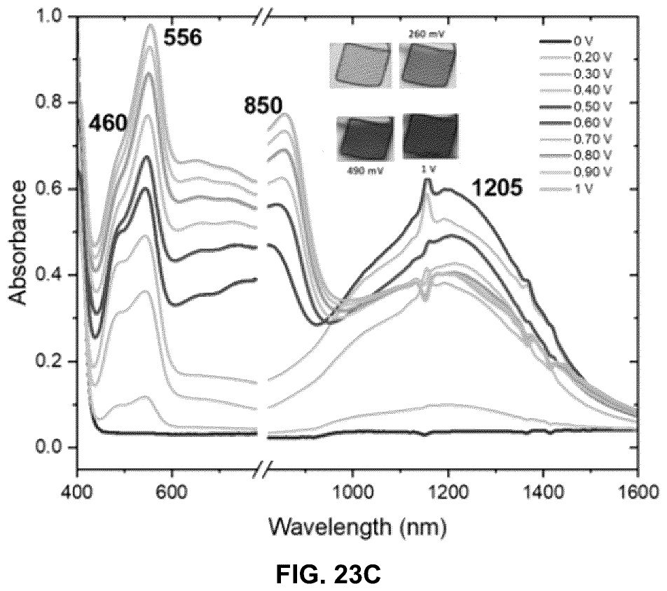

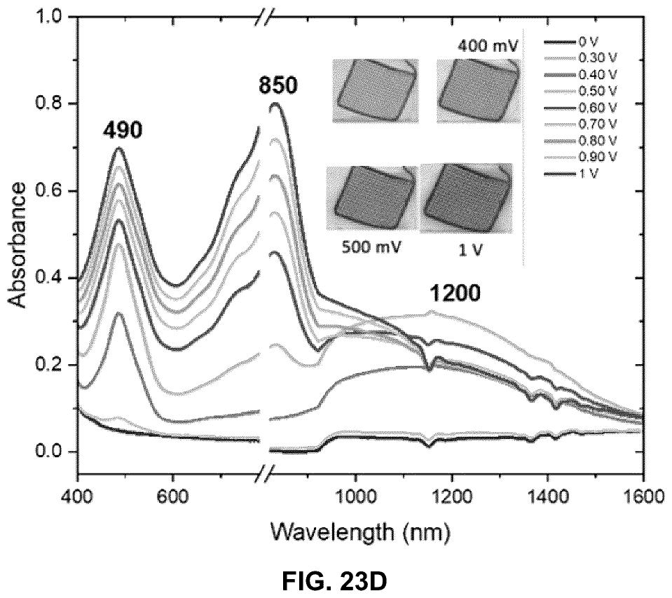

To demonstrate the electrochromic activity of these molecules, spectroelectrochemistry was conducted using an optically transparent thin layer electrode (OTTLE). All measurements were carried out at a concentration of 250 μM in 0.5 M tetrabutylammonium hexafluorophosphate (NBu₄PF₆)/dichloromethane (DCM) electrolyte. The results are depicted in FIGS. 23A-23D for TPA 1, TPA 2, TPA 3 and TPA 4. The photographic insets demonstrate the colors generated at specific applied potentials.

For all compounds, as the potential is increased, the π-π* transition for the neutral state (not shown) diminishes, while absorption peaks attributed to the cation radical as the HE and LE transitions emerge in the visible and near infrared regions, respectively. During the initial steps of applied potential, the spectra resemble those generated via initial chemical oxidation (FIGS. 22A-22D) as the cation radical is formed. Notably, two absorption peaks in the visible region, accompanied by a broad absorption in the NIR, are observed for TPA 1 (516, 710, and 1200 nm), TPA 2 (500, 542, and 1160 nm), TPA 3 (460, 556, and 1205 nm), and TPA 5 (510, 580, and 1220 nm). In contrast, a single absorption peak in the visible region, accompanied by a broad absorption in the NIR, is observed for TPA 4 (490 and 1200 nm). Photographs of the OTTLE cells during oxidation reveal distinct color changes from a colorless and transparent solution. Specifically, TPA 1, TPA 3, and TPA 5 exhibit a visible pink/magenta hue on the platinum mesh, with color intensification at higher potentials. On the other hand, TPA 2 displays a higher saturated red color, while TPA 4 appears as an orange-yellow.

As previously investigated, at higher applied potentials (typically >0.50 V), the dication absorption band is expected to emerge, indicated by a new absorption peak growing between 700-850 nm, while the absorption peak in the NIR gradually decreases. Reflecting back on the spectra in FIGS. 22A-22D from chemical oxidation using NOPF₆, the dication absorption emerges around 525-600 nm. Certain triarylamine based cation salts (e.g., MeoTPD and spiro-MeOTAD reacted with NOPF₆) exhibit an absorption at ˜700 nm due to localized HOMO-LUMO transitions of the triphenylamine moiety. However, mixed-valence compounds with two (or more) redox centers can exhibit varying behavior and therefore be categorized. These categories include 1) localized redox centers behave as separate entities, 2) intermediate coupling between mixed valence centers can exist, and 3) strong and delocalized coupling where intermediate redox states are attributed to the redox centers. An investigation was reported where the distances between redox centers in a set of triarylamine compounds ranged from a few 0.5-2 nm, demonstrated mixed-valence properties over a broad distance range. It is possible that chemical oxidation and electrochemical oxidation generates differing valence properties. However, in the case of electrochemical oxidation, a noticeable blue color is observed at the electrode for TPA 1, TPA 2 and TPA 3. While, for TPA 4 and TPA 5, a more yellow-orange is observed.

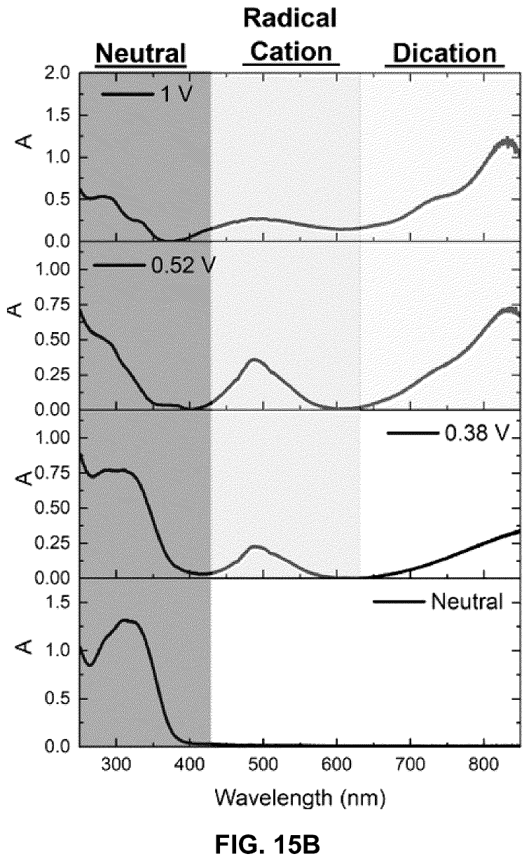

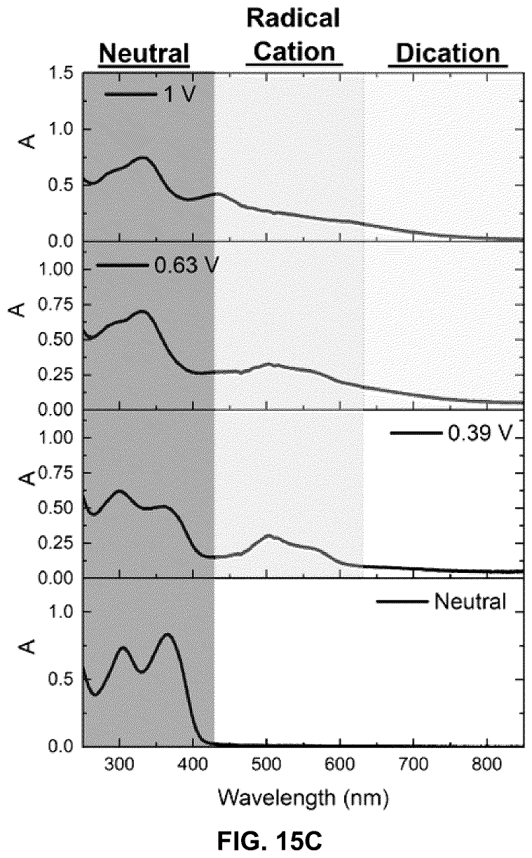

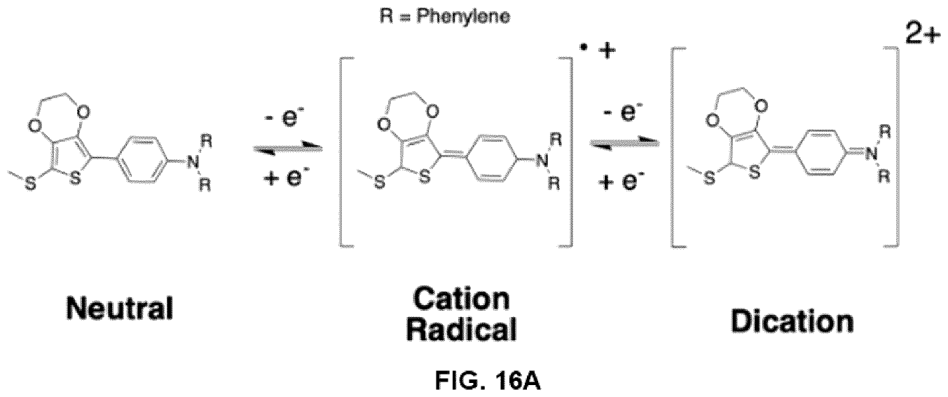

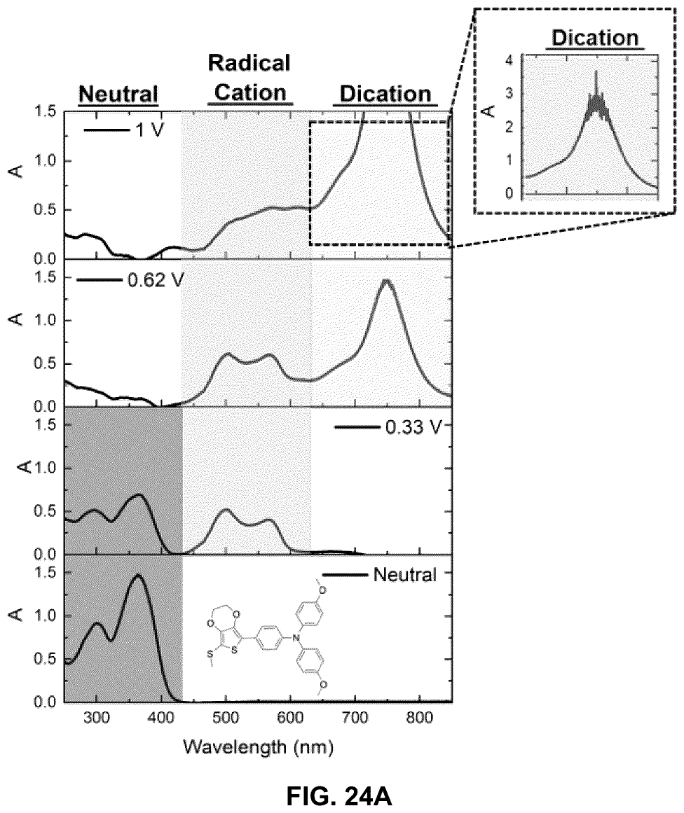

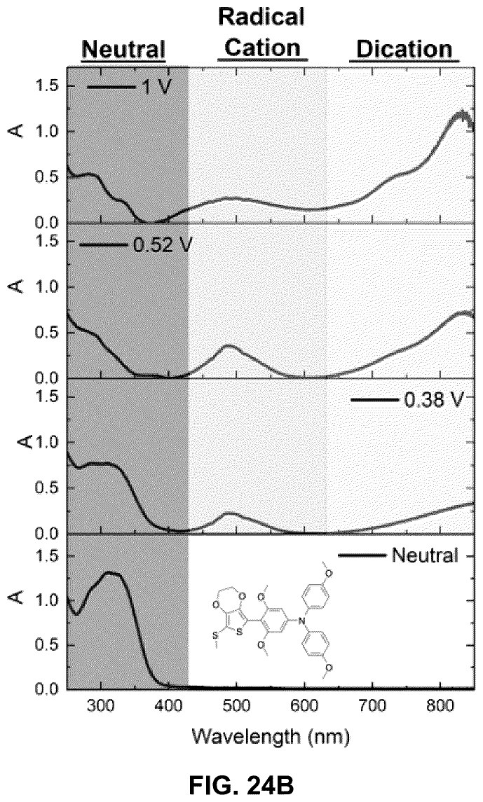

To validate the controlled formation of charge, an additional investigation involved observing the spectroelectrochemistry at low, the two E_{1/2}, and a high potential over a 10 minute duration. FIGS. 24A-24B and FIGS. 15A-15C display stacked plots showing evolution of absorption for the three states of TPA 1 compared to TPA 4. Based on the information in FIGS. 20A-20B, which illustrates the electrochemical redox evaluation, it is evident that TPA 1 displayed the largest separation of charge (ΔE=0.29 V) between the cation radical and dication, while TPA 4 displayed the smallest separation (ΔE=0.14 V). In FIGS. 24A-24B, the neutral species is used as a starting point without any applied potential and all absorption is in the UV (left dark gray band). At the first E_{1/2}=0.33 V for TPA 1, 47% of the neutral absorption intensity remains while the cation radical becomes evident in the gray band. Examining the results for TPA 4, at 0.38 V, 59% of the absorption remains. At the second E_{1/2}, both chromophores have nearly completed oxidation of the neutral species, resulting in the coexistence of cation radical and dication (light right band) species. For TPA 1 at 0.62 V, 4% of the neutral species remains, while 42% of cation radicals and 68% dications are present. Conversely, for TPA 4 at 0.52 V, 20% remaining neutral species, 26% cation radicals, and 54% dications are observed. At 1 V, no neutral species are evident for either compound. At this potential, TPA 1 retains 10% of the cation radicals with dications dominating as the primary charged species. In contrast, TPA 4 at 1 V, 20% of the cation radicals remains, while 80% of the charged species are dications. These results thus show that a larger separation of charge significantly influences the formation of each charged species in a controlled manner.

### CONCLUSION AND PERSPECTIVE

In summary, the geometric, electrochemical, optoelectronic, and colorimetric visualization of a series of ACE molecules based on thioalkyl-substituted 3,4-ethylenedioxythiophene coupled to triphenylamine units (EDOT-TPA) were compared. The phenylene ring coupled adjacently to the EDOT unit has a range in positioning and electron richness of the methoxy substituents and therefore influences the steric and electrostatic interactions. Single crystal structures allow use to determine the torsional angles and non-covalent bonding interactions. Incorporating methoxy substituents in the 2′ position or 2′,5′ results in favorable S—O and O—H interactions, which favors in planarity. While incorporating a methoxy in the 2′,6′ position results in repulsive interaction between O—O resulting in torsional strain.

Solution and electrochemical oxidation demonstrate that these class of molecules are fully transparent in the neutral state and generate vibrantly colored solutions in the generation of cation radical and dication species. Solution oxidation using Fe(OTF)₃ generates the cation radical while NOPF₆ (being a more aggressive oxidizer) generates the dication. Vibrant and varying saturation and contrast of the color magenta is accessed for most of the molecules in the cation radical state. However, 2-MeS-EDOT-2,6-dimethoxy-TPA-pOCH₃, which demonstrates repulsive strain, displayed the color orange in the series. Interestingly, further oxidation to access the dication state resulted in each molecule accessing new colors in the RGB color space where the molecule such as 2-MeS-EDOT-2-methoxy-TPA-pOCH₃ generated the color gray. Electro-oxidation successfully generated the cation radical and further higher potentials resulted in the generation of the dication species. However, selective generation and color in the formation of cation radical and dication species are further demonstrated by increasing the potential energy difference between successive charged species in a given molecular system.

Overall, this investigation serves as a comprehensive guide towards designing ACE molecules and controlling their electronic and steric properties that allows tuned control in the generation of successive charged species formation. Increasing the potential energy separation between the cation radical and dication species allows control in the generation and access of the first accessible color in the first charged state followed by the color in the second charged state that minimizes intermixing of colors that is typically observed in ACE electrochromic systems. Accessing and controlling charge separation is seen in cathodically controlled viologen systems, but has not been demonstrated for anodic coloring designs until now.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. CN103980731A, 2016, Central South University.
2. Amb, C.; et al. Navigating the Color Palette of Solution-Processable Electrochromic Polymers. Chem. Mater. 2011, 23 (3), 397-415.
3. Batsanov, S. S. Van er Waals Radii of Elements. Inorg. Mater. 2001, 37, 871-885.
4. Beaujuge, P. M.; et al. Color Control in r-Conjugated Organic Polymers for Use in Electrochromic Devices. Chem. Rev. 2010, 110 (1), 268-320.
5. Chen, J.; et al. The Effect of Different Alkyl Chains on the Photovoltaic Performance of D-rr-A Porphoryin-Sensitized Solar Cells. New J. Chem. 2015, 39, 3736-3746.
6. Christiansen, D. T. et al; 2018; Polymer Chemistry; 9:3055-3066.
7. Christiansen, D. T.; et al. New Design Paradigm for Color Control in Anodically Coloring Electrochromic Molecules. J. Am. Chem. Soc. 2019, 141(9), 3859-3862.
8. Dokid, M.; et al. Artificial Photosynthesis by Light Absorption, Charge Separation, and Multielectron Catalysis. Chem. Commun., 2018, 54, 6554-6572.
9. Fatayer, S.; et al. Molecular Structure Elucidation with Charge-State Control. Science, 2019, 365, 142-145.
10. Feng, F.; et al. An Overview of Electrochromic Devices with Electrolytes Containing Viologens. Solar Energy Materials & Solar Cells, 2023, 254, 112270.
11. He, B.; et al. ortho-Terphenylene Viologens with Through-Space Conjugation for Enhanced Photocatalytic Oxidative Coupling and Hydrogen Evolution. J. Am. Chem. Soc., 2022, 144, 4422-4430.
12. Hicks, R.; et al. Synthesis and Electronic Structure Investigations of α,ω-Bis(arylthiol)oligothiophenes: Toward Understanding Wire-Linker Interactions in Molecular-Scale Electronic Materials. J. Am. Chem. Soc., 2000, 122(28), 6746-6753.
13. Huang, M.; et al. Five-Membered-Heterocycle Bridged Viologen with High Voltage and Superior Stability for Flow Battery. Adv. Funct. Mater. 2022, 32, 2111744.
14. Imahori, H. Molecular Photoinduced Charge Separation: Fundamentals and Application. Bull. Chem. Soc. Jpn. 2023, 96, 339-352.
15. Kathiresan, M.; et al. Viologens: A Versatile Organic Molecule for Energy Storage Applications. J. Mater. Chem. A., 2021, 9, 27215-27233.
16. Kim, M.; et al. Asymmetric Molecular Modification of Viologens for Highly Stable Electrochromic Devices. RSC Adv., 2020, 10, 394-401.
17. Kirchartz, T.; et al. Classification of Solar Cells According to Mechanisms of Charge Separation and Charge Collection. Phys. Chem. Chem. Phys., 2015, 17, 4007-4014.
18. Krauss, G.; et al; Highly Efficient Doping of Conjugated Polymers Using Multielectron Acceptor Salts. Macromol. Rapid Commun. 2021, 42, 2100443.
19. Lambert, C.; et al. The Class Il/Ill Transition in Triarylamine Redox Systems. J. Am. Chem. Soc. 1999, 121 (37), 8434-8442.
20. Liou, G.-S.; et al. Synthesis and Electrochemical Properties of Novel Aromatic Poly (Amine-Amide) s with Anodically Highly Stable Yellow and Blue Electrochromic Behaviors. Macromolecules, 2009, 42 (1), 125-134.
21. Liu, L.; et al. Controlled Photoinduced Generation of “Visual” Partially and Fully Charge Separated States in Viologen Analogues. J. Am. Chem. Soc., 2021, 143 (5), 2232-2238.
22. Liu, W.; et al. A Highly Stable Neutral Viologen/Bromine Aqueous Flow Battery with High Energy and Power Density. Chem. Commun., 2019, 55, 4801.
23. Mao, L.; et al. Triphenylamine (TPA) Radical Cations and Related Macrocycles. Chinese Chemical Letters, 2021, 32, 3331-3341.
24. Nhon, L.; et al. Theory Driven Spectral Control of Bis-EDOT Arylene Radical Cation Chromophores. Chem. Mater. 2022. Accepted Manuscript.
25. Nielsen, C.; et al. Discrete Photopatternable rr-Conjugated Oligomers for Electrochromic Devices. J. Am. Chem. Soc., 2008, 130, 9734-9746.
26. Niklas, J.; et al. Photoinduced Dynamics of Charge Separation: From Photosynthesis to Polymer-Fullerene Bulk Hereojunctions, J. Phys. Chem. B, 2015, 119 (24), 7407-7416.
27. Osterholm, A. M.; et al. Conquering Residual Light Absorption in the Transmissive States of Organic Electrochromic Materials. Mater. Horiz., 2022, 9, 252-260.
28. Otero-de-la-Roza, A.; et al. Analysis of Density-Functional Errors for Noncovalent Interactions Between Charged Molecules. J. Phys. Chem. A. 2020, 124 (2), 353-361.
29. Rai, V.; et al. A Review on Recent Advances in Electrochromic Devices: A Material Approach. Adv. Eng. Mater, 2020, 22, 2000082.
30. Rettig, W. Charge Separation in Excited States of Decoupled Systems—TICT Compounds and Implications Regarding the Development of New Laser Dyes and the Primary Processes of Vision and Photosynthesis. Angew. Chem. Int. Ed. Engl. 1986, 25, 971-988.
31. Robin, M.; et al. Mixed Valence Chemistry—A Survey and Classification. Advances in Inorganic Chemistry and Radiochemistry. 1968, 10, 247-422.
32. Rojas-Gonzelez, E. A.; et al. Charge Coloration Dynamics of Electrochromic Amorphous Tungsten Oxide Studied by Simultaneous Electrochemical and Color Impedance Measurements. J. Appl. Phys., 2021, 129, 0532103.
33. Santos, W. G.; et al. Inverse Photochromism in Viologen-Tetraarylborate Ion-Pair Complexes: Optical Write/Microwave Erase Switching in Polymer Matrices. Mater. Adv. 2022, 3, 3862-3874.
34. Scherlis, D. A.; et al. π-Stacking in Charged Thiophene Oligomers. J. Phys. Chem. B. 2004, 108 (46), 17791-17795.
35. Shah, K. W.; et al. Viologen-Based Electrochromic Materials: From Small Molecules, Polymers, and Composites to their Applications. Polymers (Basel). 2019, 11 (11), 1839.
36. Shchegolkov, A. V.; et al. A Brief Overview of Electrochromic Materials and Related Devices: A Nanostructured Materials Perspective. Nanomaterials (Basel), 2021, 11 (9), 2376.
37. Sheldrick, G. M. (2015). Acta Cryst. C71, 3-8.
38. Su, Y.-S. et al., 2017, Polymers, 9:284.
39. Tamai, Y. Delocalization Boosts Charge Separation in Organic Solar Cells. Polymer Journal, 2020, 52, 691-700.
40. Tang, J. H.; et al. Multistate Redox Switching and Near-Infrared Electrochromism Based on a Star-Shaped Triruthenium Complex with a Triarylamine Core. Scientific Reports, 2016, 6, 35253.
41. Turbiez, M.; et al. Bis-EDOT End Capped by n-Hexyl or n-Hexylsulfanyl Groups: the Effect of the Substituents on the Stability of the Oxidized States. New J. Chem, 2015, 39, 1678-1684.
42. Wagner, J. S.; et al. Color Control in Bis-EDOT Phenylene Anodically Coloring Electrochromes. Chemistry of Materials, 2023, Accepted Manuscript.
43. Walczak, R. M.; et al. Poly(3,4-Alylenedioxypyrroles): The PXDOPs as Versatile yet Underutilized Electroactive and Conducting Polymers. Adv. Mater. 2006, 18 (9), 1121-1131.
44. Wu, J. T.; et al. Synthesis and Optical Properties of Redox-Active Triphenylamine-Based Derivatives with Methoxy Protecting Groups. J. Mater. Chem. C., 2018, 6, 13345-13351.
45. Wu, Y.; et al. Recent Advances in the Material Design for Intelligent Wearable Devices. Mater. Chem. Front., 2023, 7, 3278-3297.
46. Wu, Z.; et al. Self-Adaptive Switch Enabling Complete Charge Separation in Molecular-Based Optoelectronic Conversion. J. Phys. Chem. Lett. 2018, 9(4), 837-843.
47. Xie, Y. et al., 2022, Dyes and Pigments, 208:110889.
48. Yanagi, R.; et al. Charge Separation in Photocatalysts: Mechanisms, Physical Parameters, and Design Principles. ACS Energy Lett. 2022, 7 (1), 432-452.
49. Yen, H. J.; et al. Solution-Processable Triarylamine-based Electroactive High Performance Polymers for Anodically Electrochromic Applications. Polymer Chemistry, 2012, 3(2), 255-264.
50. Yokoyama, Y.; et al. A Triphenylamine Double-Decker: From A Delocalized Radical Cation to a Diradical Dication with An Excited Triplet State. Angew Chem Int. Ed Engl. 2012, 51 (37), 9403-6.
51. Zeng, J. et al., 2018, Solar Energy Materials and Solar Cells, 178:223-233.
52. Zhang, Q.; et al. Colorless-to-Colorful Switching Electrochromic Polyimides with Very High Contrast Ratio. Nature Commun, 2019, 10, 1239.
53. Zhang, S.; et al. Selenoviologen-Appendant Metallacycles with Highly Stable Radical Cations and Long-Lived Charge Separation States for Electrochromism and Photocatalysis. Angew. Chem. Int. Ed., 2022, 61, e202209054.
54. Zhao, Y. et al., 2022, Chem. Eng. J., 450:138386.

What is claimed is:

1. An anodically-coloring electrochromic molecule having a structure according to Formula 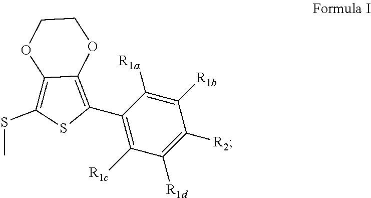
wherein R₁ₐ, R_{1b}, R₁ₑ, and R_{1d} are independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino, and
wherein R₂ is selected from a substituted or unsubstituted triphenylamine (TPA) and substituted or unsubstituted carbazole (Cz).

2. The anodically-coloring electrochromic molecule of claim 1, wherein the anodically-coloring electrochromic molecule has a structure according to Formula Ia or Formula Ib: 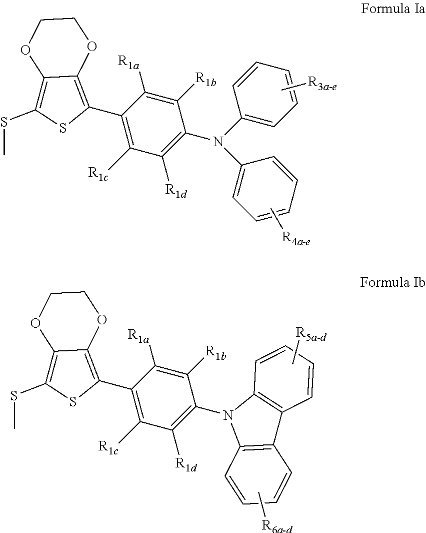
wherein, when the anodically-coloring electrochromic molecule has the structure according to Formula Ia, each of R₃ₐ₋ₑ and R₄ₐ₋ₑ is independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino; and
wherein, when the anodically-coloring electrochromic molecule has the structure according to Formula Ib, each of R_{5a-d} and R_{6a-d} is independently selected from hydrogen, alkoxy; nitro, trifluoromethyl, halogen, pseudohalogen, sulfo-oxo, thiol, oligo ether, ester, carboxylic acid, alkyl, —SCH₃, diethylphosphonite, phosphonic acid, amino, and alkylamino.

3. The anodically-coloring electrochromic molecule of claim 1, wherein each of R₁ₐ, R_{1b}, R_{1c}, and R_{1d} are independently selected from —CN, —NO₂, —CF₃, —F, —Cl, hydrogen, —CH₃, —OCH₃, —SCH₃, —NH₂, and —N(CH₃)₂.

4. The anodically-coloring electrochromic molecule of claim 1, wherein, when the anodically-coloring electrochromic molecule has the structure according to Formula Ia, each of R₃ₐ₋ₑ and R₄ₐ₋ₑ is independently selected from —CN, —NO₂, —CF₃, —F, —Cl, hydrogen, —CH₃, —OCH₃, —SCH₃, —NH₂, and —N(CH₃)₂; and
wherein, when the anodically-coloring electrochromic molecule has the structure according to Formula Ib, each of R_{5a-d} and R_{6a-d} is independently selected from —CN, —NO₂, —CF₃, —F, —C, hydrogen, —CH₃, —OCH₃, —SCH₃, —NH₂, and —N(CH₃)₂.

5. The anodically-coloring electrochromic molecule of claim 1, wherein each of R₁ₐ, R_{1b}, R_{1c}, and R_{1d} are hydrogen.

6. The anodically-coloring electrochromic molecule of claim 1, wherein R₁ₐ, R_{1b}, and R_{1d} are hydrogen and wherein R₁ₑ is methoxy.

7. The anodically-coloring electrochromic molecule of claim 1, wherein R₁ₐ and R_{1d} are hydrogen and wherein R_{1b} and R_{1c} are methoxy.

8. The anodically-coloring electrochromic molecule of claim 1, wherein R_{1b} and R_{1d} are hydrogen and wherein R₁ₐ and R_{1c} are methoxy.

9. The anodically-coloring electrochromic molecule of claim 2, wherein the anodically-coloring electrochromic molecule is Formula Ia;
wherein at least one of R₃ₐ—R₃ₑ is methoxy;
wherein at least one of R₄ₐ—R₄ₑ is methoxy; and
wherein any of R₃ₐ—R₃ₑ and any of R₄ₐ—R₄ₑ that are not methoxy are hydrogen.

10. The anodically-coloring electrochromic molecule of claim 2, wherein the anodically-coloring electrochromic molecule is Formula Ia;
wherein at least one of R₃ₐ—R₃ₑ is —SCH₃;
wherein at least one of R₄ₐ—R₄ₑ is —SCH₃; and
wherein any of R₃ₐ—R₃ₑ and any of R₄ₐ-R₄ₑ that are not —SCH₃ are hydrogen.

11. The anodically-coloring electrochromic molecule of claim 2, wherein the anodically-coloring electrochromic molecule is Formula Ia;
wherein at least one of R₃ₐ—R₃ₑ is —CN;
wherein at least one of R₄ₐ—R₄ₑ is —CN; and
wherein any of R₃ₐ—R₃ₑ and any of R₄ₐ—R₄ₑ that are not —CN are hydrogen.

12. The anodically-coloring electrochromic molecule of claim 2, wherein the anodically-coloring electrochromic molecule is Formula Ia;
wherein at least three of R₃ₐ—R₃ₑ are methoxy;
wherein at least three of R₄ₐ—R₄ₑ are methoxy; and
wherein any of R₃ₐ—R₃ₑ and any of R₄ₐ—R₄ₑ that are not methoxy are hydrogen.

13. The anodically-coloring electrochromic molecule of claim 2, wherein the anodically-coloring electrochromic molecule is Formula Ib;
wherein at least one of R₅ₐ—R_{5d} is methoxy;
wherein at least one of R₆ₐ—R_{6d} is methoxy; and
wherein any of R₅ₐ—R_{5d} and any of R₆ₐ—R_{6d} that are not methoxy are hydrogen.

14. The anodically-coloring electrochromic molecule of claim 2, wherein the anodically-coloring electrochromic molecule is Formula Ib;
wherein at least one of R₅ₐ—R_{5d} is —SCH₃;
wherein at least one of R₆ₐ—R_{6d} is —SCH₃; and
wherein any of R₅ₐ—R_{5d} and any of R₆ₐ—R_{6d} that are not —SCH₃ are hydrogen.

15. The anodically-coloring electrochromic molecule of claim 2, wherein the anodically-coloring electrochromic molecule is Formula Ib;
wherein at least one of R₅ₐ—R_{5d} is —CN;
wherein at least one of R₆ₐ—R_{6d} is —CN; and
wherein any of R₅ₐ—R_{5d} and any of R₆ₐ—R_{6d} that are not —CN are hydrogen.

16. The anodically-coloring electrochromic molecule of claim 1, wherein the anodically-coloring electrochromic molecule is selected from: 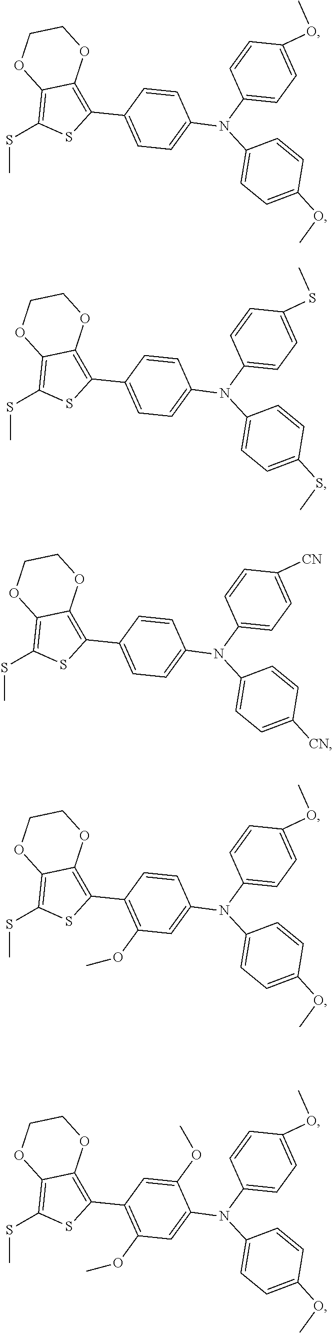 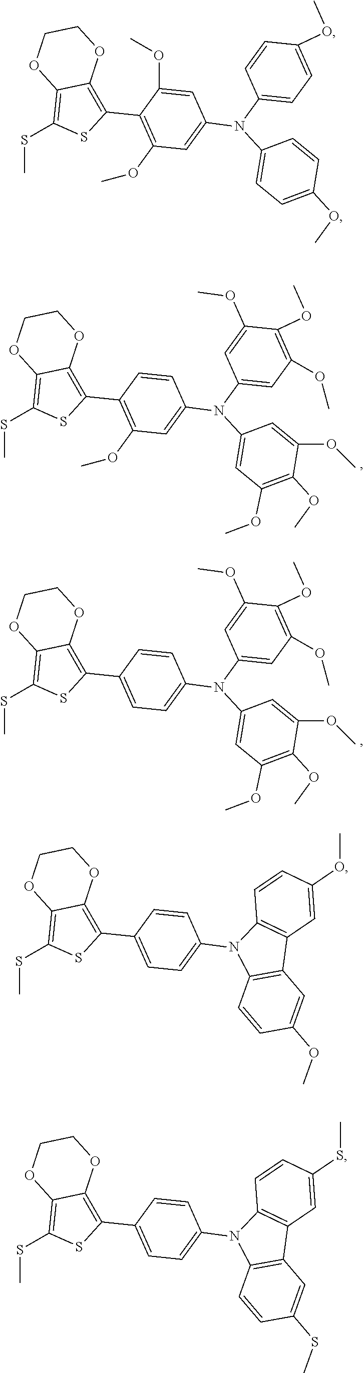 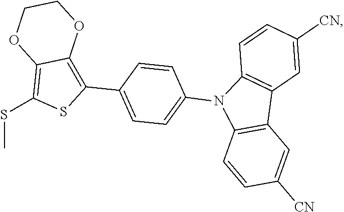 [SMILES: CSc1sc(-c2ccc(-n3c4ccc(C#N)cc4c4cc(C#N)ccc43)cc2)c2c1OCCO2]
or any combination thereof.

17. The anodically-coloring electrochromic molecule of claim 1, wherein the anodically-coloring electrochromic molecule comprises a first color in its neutral state and a second color after being oxidized.

18. The anodically-coloring electrochromic molecule of claim 17, wherein the first color is transparent.

19. The anodically-coloring electrochromic molecule of claim 17, wherein the anodically-coloring electrochromic molecule is oxidized by contact with an oxidant, by application of an oxidation potential, or any combination thereof.

20. The anodically-coloring electrochromic molecule of claim 19, wherein the oxidant comprises Fe(OTf)₃, iron (III) perchlorate, iron (III) chloride, iron (III) tosylate, nitrosonium hexafluorophosphate, 2,3,5,6-tetrafluoro-7,7,8,8-tetracyano-quinodimethane (F4TCNQ), tetracyanoquinodimethane (TCNQ), tetracyanoethylene (TCNE), [3]-radialene, tris(4-bromophenyl)ammoniumyl hexachloroantimonate (magic blue), HCl, H₂SO₄, zinc, borane, a carbonium compound, an oxonium compound, iodine, arsenic pentafluoride, or any combination thereof.
